(19) United States
(12) Patent Application Publication
(10) Pub. No.: US 2014/0235867 A1
(43) Pub. Date: Aug. 21, 2014

(54) METHOD FOR PRODUCING ALPHA-HYDROXY KETONE COMPOUND

(71) Applicants: Koji Hagiya, Osaka-shi (JP); Akio Tanaka, Osaka-shi (JP); Toshihiro Hodai, Osaka-shi (JP)

(72) Inventors: Koji Hagiya, Osaka-shi (JP); Akio Tanaka, Osaka-shi (JP); Toshihiro Hodai, Osaka-shi (JP)

(73) Assignee: SUMITOMO CHEMICAL COMPANY, LIMITED, Chuo-ku, Tokyo (JP)

(21) Appl. No.: 14/236,268

(22) PCT Filed: Aug. 27, 2012
(86) PCT No.: PCT/JP2012/072277

§ 371 Date: Jan. 30, 2014

(30) Foreign Application Priority Data
Sep. 6, 2011 (JP) 2011-193575
Jan. 10, 2012 (JP) 2012-001914
Feb. 22, 2012 (JP) 2012-035878

(51) Int. Cl. C07C 45/64; C07D 277/22; C07D 277/60; C07D 277/72; C07D 277/62; C07C 319/14; C07D 277/36

(52) U.S. Cl. CPC C07C 45/64; C07C 319/14; C07D 277/22; C07D 277/36; C07D 277/72; C07D 277/62; C07D 277/60
USPC 548/173; 568/43; 548/202; 548/182; 548/174; 548/152; 548/203; 568/386; 568/316

(57) ABSTRACT

The present invention relates to a specific thiazolium salt used for producing an α-hydroxy ketone compound, and a method for producing an α-hydroxy ketone compound by carrying out a coupling reaction of an aldehyde compound in the presence of a base compound and the specific thiazolium salt.

### TECHNICAL FIELD

The invention relates to a method for producing an α-hydroxy ketone compound, and the like.

### BACKGROUND ART

As a method for producing an α-hydroxy ketone compound by a coupling reaction of an aldehyde compound, for example, Patent Document 1 discloses a method of using a catalyst prepared from 3-ethylbenzothiazolium salt and a basic compound and a method of using a catalyst prepared from 3-benzylthiazolium salt and a basic compound.

### CITATION LIST

### Patent Literature

PLT1: JP-A-2008-44929

### SUMMARY OF INVENTION

### Technical Problem

In the present invention, the aim to be solved by the present invention is to provide an innovative method for producing an α-hydroxy ketone compound, and the like.

### Solution to Problem

In view of the above state of the art, the inventors of the present invention have made various investigations and consequently have completed the present invention.

That is, the invention is as follows.

[1] A method for producing an α-hydroxy ketone compound by carrying out a coupling reaction of an aldehyde compound in the presence of a base compound and a thiazolium salt defined by a formula (1)

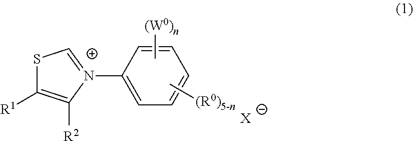

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; X⁻ denotes an anion; n denotes 1 or 2; in the case n is 2, 2 groups denoted by W⁰ may be mutually the same or different; and all or some of a plurality of groups denoted by R⁰ may be the same or different).
[2] A method for producing an α-hydroxy ketone compound by carrying out a coupling reaction of an aldehyde compound in the presence of a base compound and a thiazolium salt defined by a formula (1′)

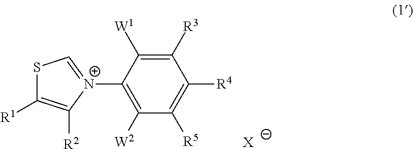

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; W¹ and W² independently denote an aryl which may have a substituent or a halogen atom; and X⁻ denotes an anion).
[3] A method for producing an α-hydroxy ketone compound by carrying out a coupling reaction of an aldehyde compound in the presence of a base compound and a thiazolium salt defined by a formula (1″)

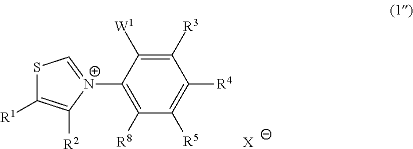

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; W¹ denotes an aryl which may have a substituent or a halogen atom; and X⁻ denotes an anion).
[4] A method for producing an α-hydroxy ketone compound by carrying out a coupling reaction of an aldehyde compound in the presence of thiazol-2-ylidene defined by a formula (1-2)

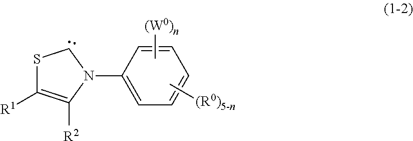

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; n denotes 1 or 2; in the case n is 2, 2 groups denoted by W⁰ may be mutually the same or different; and all or some of a plurality of groups denoted by R⁰ may be the same or different).
[5] A method for producing an α-hydroxy ketone compound by carrying out a coupling reaction of an aldehyde compound in the presence of thiazol-2-ylidene defined by a formula (1′-2)

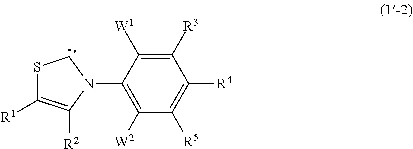

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; and W¹ and W² independently denote an aryl which may have a substituent or a halogen atom).
[6] A method for producing an α-hydroxy ketone compound by carrying out a coupling reaction of an aldehyde compound in the presence of thiazol-2-ylidene defined by a formula (1″-2)

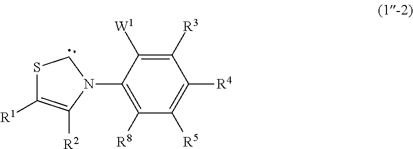

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; and W¹ denotes an aryl which may have a substituent or a halogen atom).
[7] The method as described in one of [1] to [6] in which the coupling reaction of an aldehyde compound is carried out in the presence of carbon dioxide.
[8] The method as described in one of [1] to [7] in which the base compound is at least one kind of compound selected from the group consisting of organic bases, alkali metal salts, and alkaline earth metal salts.
[9] The method as described in one of [1] to [8] in which the coupling reaction of an aldehyde compound is a homo-coupling reaction of an aldehyde compound defined by a formula (2)

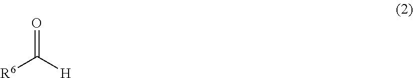

(wherein R⁶ denotes a hydrogen atom, an alkyl which may have a substituent, an aryl which may have a substituent, or a heteroaryl which may have a substituent).
[10] The method as described in one of [1] to [8] in which the coupling reaction of an aldehyde compound is a cross-coupling reaction of an aldehyde compound defined by a formula (2)

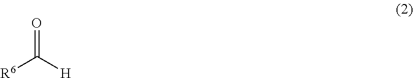

(wherein R⁶ denotes a hydrogen atom, an alkyl which may have a substituent, an aryl which may have a substituent, or a heteroaryl which may have a substituent) and an aldehyde compound defined by a formula (4)

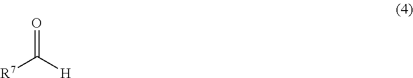

(wherein R⁷ is different from R⁶ and denotes a hydrogen atom, an alkyl which may have a substituent, an aryl which may have a substituent, or a heteroaryl which may have a substituent).
[11] The method as described in [10] in which R⁶ denotes an alkyl which may have a substituent and R⁷ denotes a hydrogen atom.
[12] The method as described in [11] in which the aldehyde compound defined by the formula (4) is formaldehyde co-existing with water.
[13] The method as described in [12] in which the coupling reaction of an aldehyde compound is carried out in the presence of a solvent having no compatibility with water.
[14] The method as described in one of [10] to [13] in which the aldehyde compound defined by the formula (2) is 3-methylthiopropanal and the α-hydroxy ketone compound is 4-methylthio-2-oxo-1-butanol.
[15] A thiazolium salt defined by a formula (1)

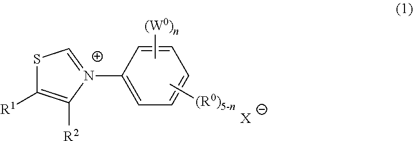

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; X⁻ denotes an anion; n denotes 1 or 2; in the case n is 2, 2 groups denoted by W⁰ may be mutually the same or different; and all or some of a plurality of groups denoted by R⁰ may be the same).
[16] A thiazolium salt defined by a formula (1′)

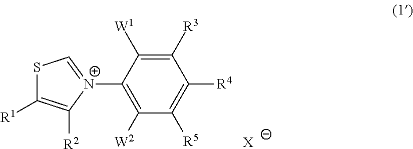

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R² and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; W¹ and W² independently denote an aryl which may have a substituent or a halogen atom; and X⁻ denotes an anion).
[17] The thiazolium salt as described in [16] in which R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom, a halogen atom, or a C₁₋₁₀ alkyl; and
W¹ and W² independently denote a C₆₋₁₀ aryl which may have a substituent.
[18] A thiazolium salt defined by a formula (1″)

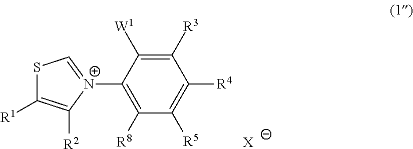

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; W⁴ denotes an aryl which may have a substituent or a halogen atom; and X⁻ denotes an anion).
[19] The thiazolium salt as described in [18] in which R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom or a C₁₋₁₀ alkyl;
R⁸ denotes a C₁₋₁₀ alkyl; and
W¹ denotes a C₆₋₁₀ aryl which may have a substituent.
[20] 3-[2,6-di(phenyl)phenyl]-4,5-dimethylthiazolium salt, 3-[2,6-bis(3,5-di-tert-butylphenyl)phenyl]-4,5-dimethylthiazolium salt, 3-[(2,6-diphenyl-3,5-diisopropyl)phenyl]-4,5-dimethylthiazolium salt, 3-[2,6-bis(3,5-di-tert-butylphenyl)phenyl]-4,5,6,7-tetrahydrobenzothiazolium salt, 3-[2,6-bis(3,5-di-tert-butylphenyl)-4-chlorophenyl]-4,5,6,7-tetrahydrobenzothiazolium salt, 3-[2-(3,5-di-tert-butylphenyl)-6-methylphenyl]-4,5,6,7-tetrahydrobenzothiazolium salt, or 3-[2,6-bis(3,5-di-tert-butylphenyl)phenyl]-4,5,6,7-tetrahydro-7-ethylbenzothiazolium salt.
[21] A thiazol-2-ylidene defined by a formula (1-2)

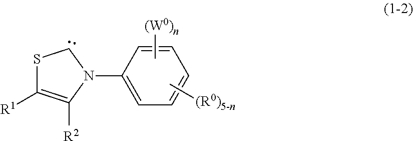

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; n denotes 1 or 2; in the case n is 2, 2 groups denoted by W⁰ may be mutually the same or different; and all or some of a plurality of groups denoted by R⁰ may be the same).
[22] A thiazol-2-ylidene defined by a formula (1′-2)

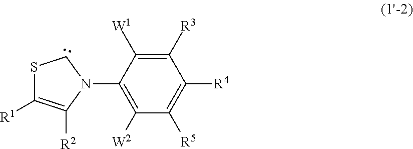

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; and W¹ and W² independently denote an aryl which may have a substituent or a halogen atom).
[23] The thiazol-2-ylidene as described in [22] in which R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom, a halogen atom, or a C₁₋₁₀ alkyl; and
W¹ and W² independently denote a C₆₋₁₀ aryl which may have a substituent.
[24] A thiazol-2-ylidene defined by a formula (1″-2)

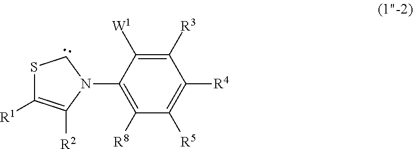

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; and W⁴ denotes an aryl which may have a substituent or a halogen atom).
[25] The thiazol-2-ylidene as described in [24] in which R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom or a C₁₋₁₀ alkyl;
R⁸ denotes a C₁₋₁₀ alkyl; and
W¹ denotes a C₆₋₁₀ aryl which may have a substituent.
[26] 3-[2,6-di(phenyl)phenyl]-4,5-dimethylthiazol-2-ylidene, 3-[2,6-bis(3,5-di-tert-butylphenyl)phenyl]-4,5-dimethylthiazol-2-ylidene, 3-[(2,6-diphenyl-3,5-diisopropyl)phenyl]-4,5-dimethylthiazol-2-ylidene, 3-[2,6-bis(3,5-di-tert-butylphenyl)phenyl]-4,5,6,7-tetrahydrobenzothiazol-2-ylidene, 3-[2,6-bis(3,5-di-tert-butylphenyl)-4-chlorophenyl]-4,5,6,7-tetrahydrobenzothiazol-2-ylidene, 3-[2-(3,5-di-tert-butylphenyl)-6-methylphenyl]-4,5,6,7-tetrahydrobenzothiazol-2-ylidene, or 3-[2,6-bis(3,5-di-tert-butylphenyl)phenyl]-4,5,6,7-tetrahydro-7-ethylbenzothiazol-2-ylidene.
[27] A method for producing a thiazolium salt defined by a formula (8)

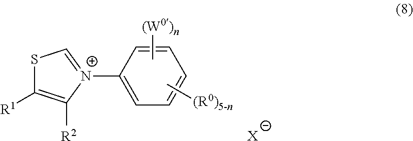

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; all or some of 4 groups denoted by R⁰ may be the same; W⁰′ denotes an aryl which may have a substituent; X⁻ denotes an anion; and n denotes the same as above), which comprises

a step A of obtaining a 3-(aryl-substituted aryl)-2-thiazole-thione compound defined by a formula (7)

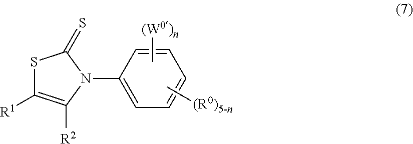

(wherein R¹, R², R⁰, W⁰′, and n respectively denote the same as described above) by a coupling reaction of a 3-(halo-substituted aryl)-2-thiazole-thione compound defined by a formula (6)

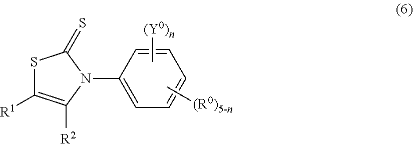

(wherein R¹, R², R⁰, and X⁻ respectively denote the same as described above; and Y⁰ denotes a halogen atom) and an aryl compound defined by a formula (5)

W⁰′-L (5)

(wherein W⁰′ denotes the same as described above; and L denotes a leaving group)
in the presence of a palladium catalyst; and

a step B of oxidizing the 3-(aryl-substituted aryl)-2-thiazole-thione compound defined by the formula (7) obtained in the step A.

[28] The method as described in [27] in which L is —B(OH)₂ or —MgX⁰ (wherein X⁰ denotes a halogen atom).
[29] The method as described in [27] in which L is —B(OH)₂ and the coupling reaction in the step A is carried out in the presence of a base compound.
[30] The method as described in [27] in which L is —MgX⁰ (wherein X⁰ denotes a halogen atom) and the coupling reaction in the step A is carried out in the presence of a zinc compound.
[31] A thiazole-thione compound defined by a formula (7′)

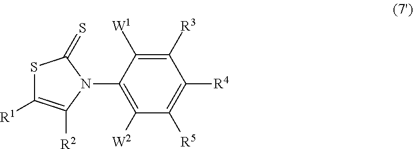

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; and W¹ and W² independently denote an aryl which may have a substituent or a halogen atom).
[32] The thiazole-thione compound as described in [31] in which R¹ and R² independently denote a C₁₋₁₀ alkyl, or R¹ and R² are bonded to each other to form a C₅₋₂₀ cycloalkene ring together with the carbon atoms bonded to R¹ and R²;
R³, R⁴, and R⁵ independently denote a hydrogen atom, a halogen atom, or a C₁₋₁₀ alkyl; and
W¹ and W² independently denote a C₆₋₂₀ aryl.
[33] A thiazole-thione compound defined by a formula (7″)

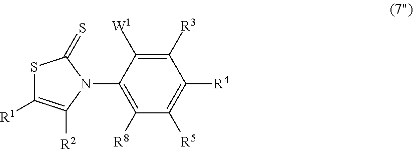

(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; and W¹ denotes an aryl which may have a substituent or a halogen atom).
[34] The thiazole-thione compound as described in [33] in which R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom or a C₁₋₁₀ alkyl;
R⁸ denotes a C₁₋₁₀ alkyl; and
W¹ denotes a C₆₋₂₀ aryl.

### Advantageous Effects of Invention

The present invention provides an innovative method for producing an α-hydroxy ketone compound, and the like. The present invention is advantageous in terms of improvement of selectivity in production of an α-hydroxy ketone compound per unit catalyst amount.

### DESCRIPTION OF EMBODIMENTS

Hereinafter, the present invention will be described in detail.

The present invention is characterized by carrying out a coupling reaction (hereinafter, sometimes referred to as the present reaction) of an aldehyde compound in the presence of a base compound and a thiazolium salt defined by a formula (1)

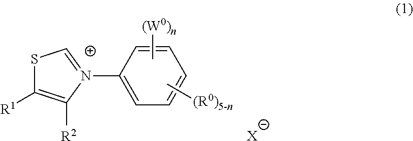

(hereinafter, sometimes referred to as thiazolium salt (1)).

The thiazolium salt (1) is preferably a thiazolium salt defined by a formula (1′)

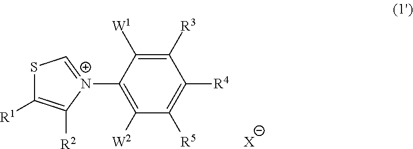

(hereinafter, sometimes referred to as thiazolium salt (1′)) or a thiazolium salt defined by a formula (1″)

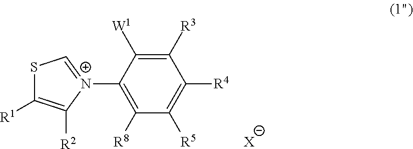

(hereinafter, sometimes referred to as thiazolium salt (1″)).

Examples of an alkyl denoted by R¹ and R² may be C₁₋₁₀ straight chain, branched chain, and cyclic alkyl such as methyl, ethyl, propyl, isopropyl, butyl, isobutyl, sec-butyl, tert-butyl, pentyl, decyl, cyclopropyl, 2,2-dimethylcyclopropyl, cyclopentyl, cyclohexyl, and menthyl.

Examples of a substituent which the alkyl denoted by R¹ and R² may have may be a C₆₋₁₀ aryl, which may have a C₁₋₁₀ alkoxy, such as phenyl, naphthyl, 4-methylphenyl, and 4-methoxyphenyl; a C₁₋₁₀ alkoxy, which may have a fluorine atom, such as methoxy, ethoxy, propoxy, isopropoxy, butoxy, isobutoxy, sec-butyloxy, tert-butoxy, and trifluoromethoxy; a C₆₋₁₀ aryl-containing C₁₋₁₀ alkoxy, which may have a C₁₋₁₀ alkoxy, such as benzyloxy, 4-methylbenzyloxy, and 4-methoxybenzyloxy; a C₁₋₁₀ alkoxy having a C₆₋₁₀ aryloxy-containing C₆₋₁₀ aryl such as 3-phenoxybenzyloxy; a C₆₋₁₀ aryloxy, which may have a C₁₋₁₀ alkoxy, such as phenoxy, 2-methylphenoxy, 4-methylphenoxy, and 4-methoxyphenoxy; a C₆₋₁₀ aryloxy-containing C₆₋₁₀ aryloxy such as 3-phenoxyphenoxy; a C₂₋₁₀ acyl, which may have a C₁₋₁₀ alkoxy, such as acetyl, propionyl, benzylcarbonyl, 4-methylbenzylcarbonyl, 4-methoxybenzylcarbonyl, benzoyl, 2-methylbenzoyl, 4-methylbenzoyl, and 4-methoxybenzoyl; carboxy; and a fluorine atom.

Examples of an alkyl having a substituent and denoted by R¹ and R² may be fluoromethyl, trifluoromethyl, methoxymethyl, ethoxymethyl, methoxyethyl, benzyl, 4-fluorobenzyl, 4-methylbenzyl, phenoxymethyl, 2-oxopropyl, 2-oxobutyl, phenacyl, and 2-carboxyethyl.

Examples of an aryl denoted by R¹ and R² may be a C₆₋₁₀ aryl such as phenyl, 2-methylphenyl, 4-methylphenyl, and naphthyl.

Examples of a substituent which the aryl may have may be a C₁₋₁₀ alkyl containing a C₁₋₁₀ alkoxy or a fluorine atom such as fluoromethyl, trifluoromethyl, methoxymethyl, ethoxymethyl, and methoxyethyl; a C₁₋₁₀ alkoxy, which may have a C₁₋₁₀ alkoxy or a fluorine atom such as methoxy, ethoxy, propoxy, isopropoxy, butoxy, isobutyloxy, sec-butyloxy, tert-butyloxy, pentyloxy, cyclopentyloxy, fluoromethoxy, trifluoromethoxy, methoxymethoxy, ethoxymethoxy, and methoxyethoxy; and a halogen atom such as a fluorine atom and a chlorine atom.

Examples of an aryl having a substituent may be 4-chlorophenyl and 4-methoxyphenyl.

Examples of an alkoxycarbonyl denoted by R¹ and R² may be C₂₋₁₁ straight chain, branched chain, and cyclic alkoxycarbonyl such as methoxycarbonyl, ethoxycarbonyl, n-propoxycarbonyl, n-butoxycarbonyl, sec-butoxycarbonyl, tert-butoxycarbonyl, pentyloxycarbonyl, decyloxycarbonyl, cyclopropoxycarbonyl, and cyclohexyloxycarbonyl.

Examples of a substituent which the alkoxycarbonyl denoted by R¹ and R² may have may be a C₆₋₁₀ aryl, which may have a C₁₋₁₀ alkoxy, such as phenyl, naphthyl, 4-methylphenyl, and 4-methoxyphenyl; a C₁₋₁₀ alkoxy, which may have a fluorine atom, such as methoxy, ethoxy, propoxy, isopropoxy, butoxy, isobutoxy, sec-butoxy, tert-butoxy, and trifluoromethoxy; a C₆₋₁₀ aryl-containing C₁₋₁₀ alkoxy, which may have a C₁₋₁₀ alkoxy, such as benzyloxy, 4-methylbenzyloxy, and 4-methoxybenzyloxy; a C₁₋₁₀ alkoxy having a C₆₋₁₀ aryloxy-containing C₆₋₁₀ aryl such as 3-phenoxybenzyloxy; a C₆₋₁₀ aryloxy, which may have a C₁₋₁₀ alkoxy, such as phenoxy, 2-methylphenoxy, 4-methylphenoxy, and 4-methoxyphenoxy; a C₆₋₁₀ aryloxy-containing C₆₋₁₀ aryloxy such as 3-phenoxyphenoxy; a C₂₋₁₀ acyl, which may have a C₁₋₁₀ alkoxy, such as acetyl, propionyl, benzylcarbonyl, 4-methylbenzylcarbonyl, 4-methoxybenzylcarbonyl, benzoyl, 2-methylbenzoyl, 4-methylbenzoyl, and 4-methoxybenzoyl; and a fluorine atom.

Examples of an alkoxycarbonyl having a substituent and defined by R¹ and R² may be fluoromethoxycarbonyl, trifluoromethoxycarbonyl, methoxymethoxycarbonyl, ethoxymethoxycarbonyl, benzyloxycarbonyl, 4-fluorobenzyloxycarbonyl, 4-methylbenzyloxycarbonyl, phenoxymethoxycarbonyl, 2-oxopropoxycarbonyl, and 2-oxobutoxycarbonyl.

Examples of an alkylcarbonyl denoted by R¹ and R² may be C₂₋₁₁ straight chain, branched chain, and cyclic alkylcarbonyl such as acetyl, ethylcarbonyl, propylcarbonyl, butylcarbonyl, sec-butylcarbonyl, tert-butylcarbonyl, pentylcarbonyl, decylcarbonyl, cyclopropylcarbonyl, and cyclohexylcarbonyl.

Examples of a substituent which an alkylcarbonyl denoted by R¹ and R² may have may be a C₆₋₁₀ aryl, which may have a C₁₋₁₀ alkoxy, such as phenyl, naphthyl, 4-methylphenyl, and 4-methoxyphenyl; a C₁₋₁₀ alkoxy, which may have a fluorine atom, such as methoxy, ethoxy, propoxy, isopropoxy, butoxy, isobutoxy, sec-butoxy, tert-butoxy, trifluoromethoxy, sec-butyloxy, tert-butyloxy, and trifluoromethyloxy; a C₆₋₁₀ aryl-containing C₁₋₁₀ alkoxy, which may have a C₁₋₁₀ alkoxy, such as benzyloxy, 4-methylbenzyloxy, and 4-methoxybenzyloxy; a C₁₋₁₀ alkoxy having a C₆₋₁₀ aryloxy-containing C₆₋₁₀ aryl such as 3-phenoxybenzyloxy; a C₆₋₁₀ aryloxy, which may have a C₁₋₁₀ alkoxy, such as phenoxy, 2-methylphenoxy, 4-methylphenoxy, and 4-methoxyphenoxy; a C₆₋₁₀ aryloxy-containing C₆₋₁₀ aryloxy such as 3-phenoxyphenoxy; a C₂₋₁₀ acyl, which may have a C₁₋₁₀ alkoxy, such as acetyl, propionyl, benzylcarbonyl, 4-methylbenzylcarbonyl, 4-methoxybenzylcarbonyl, benzoyl, 2-methylbenzoyl, 4-methylbenzoyl, and 4-methoxybenzoyl; and a fluorine atom.

Examples of an alkylcarbonyl having a substituent and defined by R¹ and R² may be fluoromethylcarbonyl, trifluoromethylcarbonyl, methoxymethylcarbonyl, ethoxymethylcarbonyl, benzylcarbonyl, 4-fluorobenzylcarbonyl, 4-methylbenzylcarbonyl, phenoxymethylcarbonyl, 2-oxopropylcarbonyl, and 2-oxobutylcarbonyl.

R¹ and R² may be bonded to each other to form a ring together with the carbon atoms bonded to R¹ and R² and examples of the ring may be cycloalkene rings such as a cyclopentene, a cyclohexene, and a cycloheptene. These rings may be substituted with a substituent which the alkyl may have in the case R¹ and R² do not form a ring.

Examples of an alkyl denoted by R⁰, R³, R⁴, R⁵, and R⁸ may be C₁₋₁₀ straight chain, branched chain, and cyclic alkyl such as methyl, ethyl, propyl, isopropyl, butyl, isobutyl, sec-butyl, tert-butyl, pentyl, decyl, cyclopropyl, 2,2-dimethylcyclopropyl, cyclopentyl, cyclohexyl, and menthyl.

Examples of a substituent which the alkyl denoted by R⁰, R³, R⁴, R⁵, and R⁸ may have may be a C₆₋₁₀ aryl, which may have a C₁₋₁₀ alkoxy, such as phenyl, naphthyl, 4-methylphenyl, and 4-methoxyphenyl; a C₁₋₁₀ alkoxy, which may have a fluorine atom, such as methoxy, ethoxy, propoxy, isopropoxy, butoxy, isobutoxy, sec-butoxy, tert-butoxy, and trifluoromethoxy; a C₆₋₁₀ aryl-containing C₁₋₁₀ alkoxy, which may have a C₁₋₁₀ alkoxy, such as benzyloxy, 4-methylbenzyloxy, and 4-methoxybenzyloxy; a C₁₋₁₀ alkoxy having a C₆₋₁₀ aryloxy-containing C₆₋₁₀ aryl such as 3-phenoxybenzyloxy; a C₆₋₁₀ aryloxy, which may have a C₁₋₁₀ alkoxy, such as phenoxy, 2-methylphenoxy, 4-methylphenoxy, and 4-methoxyphenoxy; a C₆₋₁₀ aryloxy-containing C₆₋₁₀ aryloxy such as 3-phenoxyphenoxy; a C₂₋₁₀ acyl, which may have a C₁₋₁₀ alkoxy, such as acetyl, propionyl, benzylcarbonyl, 4-methylbenzylcarbonyl, 4-methoxybenzylcarbonyl, benzoyl, 2-methylbenzoyl, 4-methylbenzoyl, and 4-methoxybenzoyl; carboxy; and a fluorine atom.

Examples of an alkyl having a substituent and denoted by R⁰, R³, R⁴, R⁵, and R⁸ may be fluoromethyl, trifluoromethyl, methoxymethyl, ethoxymethyl, methoxyethyl, benzyl, 4-fluorobenzyl, 4-methylbenzyl, phenoxymethyl, 2-oxopropyl, 2-oxobutyl, phenacyl, and 2-carboxyethyl.

Examples of an aryl denoted by R⁰, R³, R⁴, and R⁵ may be a C₆₋₁₀ aryl such as phenyl, 2-methylphenyl, 4-methylphenyl, and naphthyl.

Examples of a substituent which the aryl may have may be a C₁₋₁₀ alkyl containing a C₁₋₁₀ alkoxy or a fluorine atom such as fluoromethyl, trifluoromethyl, methoxymethyl, ethoxymethyl, and methoxyethyl; a C₁₋₁₀ alkoxy which may have a C₁₋₁₀ alkoxy or a fluorine atom such as methoxy, ethoxy, propoxy, isopropoxy, butoxy, isobutoxy, sec-butoxy, tert-butoxy, pentyloxy, cyclopentyloxy, fluoromethoxy, trifluoromethoxy, methoxymethoxy, ethoxymethoxy, and methoxyethoxy; and a halogen atom such as a fluorine atom and a chlorine atom.

Examples of an aryl having a substituent may be 4-chlorophenyl and 4-methoxyphenyl.

Examples of a halogen atom denoted by R⁰, R³, R⁴, and R⁵ may be a fluorine atom, a chlorine atom, a bromine atom, and an iodine atom.

W¹ and W² independently denote an aryl which may have a substituent or a halogen atom.

Examples of an aryl denoted by W⁰, W¹, and W² may be a C₆₋₂₀ aryl such as phenyl, naphthyl, anthryl, and phenanthryl.

Examples of a substituent which the aryl may have are not particularly limited if they do not inhibit the present reaction and may include an alkyl which may have a substituent, an aryl which may have a substituent, an alkoxy which may have a substituent, a nitro group; a cyano group; a C₂₋₁₀ alkoxycarbonyl such as methoxycarbonyl and ethoxycarbonyl; an acyl such as formyl, acetyl, and propionyl; a sulfo group; and a halogen atom such as a fluorine atom, a chlorine atom, a bromine atom, and an iodine atom.

Examples of an aryl which may have a substituent denoted by W⁰, W¹, and W² may be a C₆₋₂₀ aryl such as 2-fluorophenyl, 2-nitronaphthyl, 2-cyanophenyl, 4-nitrophenyl, 2,6-dichlorophenyl, 2,4,6-tribromophenyl, 3,5-bis(trifluoromethyl)phenyl, 2-methylphenyl, 4-methylphenyl, 3,5-dimethylphenyl, 3,5-di-tert-butylphenyl, and 3,5-diphenylphenyl.

Examples of a halogen atom denoted by W⁰, W¹, and W² may be a fluorine atom, a chlorine atom, a bromine atom, and an iodine atom.

In the above-mentioned formula (1), it is preferable that R¹ and R² are independently a C₁₋₁₀ (more preferably a C₁₋₄) alkyl, or that R¹ and R² are bonded to each other to form a C₅₋₇ cycloalkene ring (more preferably a cyclohexene ring) together with the carbon atoms bonded to R¹ and R²; it is more preferable that the carbon atom of the cycloalkene ring in the thiazolium ring S side (the 7th position in the cyclohexene ring) has a C₁₋₄ alkyl as a substituent; it is preferable that R⁰ is a hydrogen atom, a halogen atom, or a C₁₋₁₀ alkyl; and it is preferable that W⁰ is a C₆₋₁₀ aryl which may have a substituent. The aryl is more preferably phenyl having a substituent, even more preferably phenyl having a bulky group at one of the 3rd and the 5th positions, and still more preferably phenyl having a bulky group at the 3rd and the 5th positions. Examples of the bulky group may be phenyl, trifluoromethyl, tert-butyl, a chlorine atom, a bromine atom, an iodine atom, nitro, cyano, methoxycarbonyl, acyl, sulfo, and 3,4,5-trifluorophenyl.

In the above-mentioned formula (1′), it is preferable that R¹ and R² are independently a C₁₋₁₀ (more preferably a C₁₋₄) alkyl, or that R¹ and R² are bonded to each other to form a C₅₋₇ cycloalkene ring (more preferably a cyclohexene ring) together with the carbon atoms bonded to R¹ and R²; it is more preferable that the carbon atom of the cycloalkene ring in the thiazolium ring S side (the 7th position in the cyclohexene ring) has a C₁₋₄ alkyl as a substituent; it is preferable that R³, R⁴, and R⁵ are independently a hydrogen atom, a halogen atom, or a C₁₋₁₀ alkyl; and it is preferable that W¹ and W² are independently a C₆₋₁₀ aryl which may have a substituent. The aryl is more preferably phenyl having a substituent, even more preferably phenyl having a bulky group at one of the 3rd and the 5th positions, and still more preferably phenyl having a bulky group at the 3rd and the 5th positions. Examples of the bulky group may be phenyl, trifluoromethyl, tert-butyl, a chlorine atom, a bromine atom, an iodine atom, nitro, cyano, methoxycarbonyl, acyl, sulfo, and 3,4,5-trifluorophenyl.

In the above-mentioned formula (1″), it is preferable that R¹ and R² are independently a Cₒ (more preferably a C₁₋₄) alkyl, or that R¹ and R² are bonded to each other to form a C₅₋₇ cycloalkene ring (more preferably a cyclohexene ring) together with the carbon atoms bonded to R¹ and R²; it is more preferable that the carbon atom of the cycloalkene ring in the thiazolium ring S side (the 7th position in the cyclohexene ring) has a C₁₋₄ alkyl as a substituent; it is preferable that R³, R⁴, and R⁵ are independently a hydrogen atom or a C₁₋₁₀ alkyl; it is preferable that R⁸ is a C₁₋₁₀ alkyl; and it is preferable that W¹ is a C₆₋₁₀ aryl which may have a substituent. The aryl is more preferably phenyl having a substituent, even more preferably phenyl having a bulky group at one of the 3rd and the 5th positions, and still more preferably phenyl having a bulky group at the 3rd and the 5th positions. Examples of the bulky group may be phenyl, trifluoromethyl, tert-butyl, a chlorine atom, a bromine atom, an iodine atom, nitro, cyano, methoxycarbonyl, acyl, sulfo, and 3,4,5-trifluorophenyl.

Examples of the anion denoted by X, that is, a monovalent anion, may be a halide ion such as chloride ion, bromide ion, and iodide ion; an alkanesulfonate ion which may have a fluorine atom such as methanesulfonate and trifluoromethanesulfonate; acetate ion which may have a halogen atom such as trifluoroacetate and trichloroacetate ion; nitrate ion; perchlorate ion; a tetrahaloborate ion such as tetrafluoroborate and tetrachloroborate; a hexahalophosphate ion such as hexafluorophosphate; a hexahaloantimonate ion such as hexafluoroantimonate and hexachloroantimonate; a pentahalostannate ion such as pentafluorostannate and pentachlorostannate; and a tetraarylborate which may have a substituent such as tetraphenyl borate, tetrakis(pentafluorophenyl) borate, and tetrakis[3,5-bis(trifluoromethyl)phenyl] borate.

Examples of a thiazolium salt (1′) may be 3-[2,6-di(phenyl)phenyl]-thiazolium chloride, 3-[2,6-di(phenyl)phenyl]-4,5-dimethylthiazolium chloride, 3-[2,6-di(phenyl)phenyl]-4,5-diethylthiazolium chloride, 3-[2,6-di(phenyl)phenyl]-4-methyl-5-acylthiazolium chloride, 3-[2,6-di(phenyl)phenyl]-4-methyl-5-methoxycarbonylthiazolium chloride, 3-[2,6-bis(3,5-di-tert-butylphenyl)phenyl]-thiazolium chloride, 3-[2,6-bis(3,5-di-tert-butylphenyl)phenyl]-4,5-dimethylthiazolium chloride, 3-[2,6-bis(3-tert-butylphenyl)phenyl]-4,5-dimethylthiazolium chloride, 3-[2,6-bis(3,5-di-tert-butylphenyl)-4-methylphenyl]-4,5-dimethylthiazolium chloride, 3-[2,6-bis(3,5-di-tert-butylphenyl)-4-chlorophenyl]-4,5-dimethylthiazolium chloride, 3-[2,6-bis(3,5-dibromophenyl)phenyl]-4,5-dimethylthiazolium chloride, 3-[2,6-bis(3,5-dichlorophenyl)phenyl]-4,5-dimethylthiazolium chloride, 3-[2,6-bis(3,5-di-tert-butylphenyl)phenyl]-4,5-diethylthiazolium chloride, 3-[2,6-bis(3,5-di-tert-butylphenyl)phenyl]-4-methyl-5-acylthiazolium chloride, 3-[2,6-bis(3,5-di-tert-butylphenyl)phenyl]-4-methyl-5-ethoxycarbonylthiazolium chloride, 3-[2,6-bis[3,5-di-trifluoromethylphenyl)phenyl]-4,5-dimethylthiazolium chloride, 3-[2,6-bis[3,5-di-trifluoromethylphenyl)phenyl]-4,5-diethylthiazolium chloride, 3-[2,6-bis[3,5-di-methoxyphenyl)phenyl]-4,5-dimethylthiazolium chloride, 3-[2,6-bis[3,5-di-methoxyphenyl)phenyl]-4,5-diethylthiazolium chloride, 3-[2,6-bis[3,5-di-methylphenyl)phenyl]-4,5-dimethylthiazolium chloride, 3-[2,6-bis[3,5-di-methylphenyl)phenyl]-4,5-diethylthiazolium chloride, 3-[(2,6-diphenyl-3,5-diisopropyl)phenyl]-thiazolium chloride, 3-[(2,6-diphenyl-3,5-diisopropyl)phenyl]-4,5-dimethylthiazolium chloride, 3-[(2,6-diphenyl-3,5-diisopropyl)phenyl]-4,5-diethylthiazolium chloride, 3-[2,6-bis(3,5-dinitrophenyl)phenyl]-4,5-dimethylthiazolium chloride, 3-[(2,6-diphenyl-3,5-dimethyl)phenyl]-4,5-dimethylthiazolium chloride, 3-[2,6-bis(3,5-dibromophenyl)phenyl]-4,5-dimethylthiazolium chloride, 3-[(2,6-bis-(3,5-dichlorophenyl)-3,5-diisopropyl)phenyl]-4,5-dimethylthiazolium chloride, 3-[2,6-bis(3,5-di-tert-butylphenyl)phenyl]-5-n-butylthiazolium chloride, 3-[2,6-bis(3,5-di-tert-butylphenyl)phenyl]-5-tert-butylthiazolium chloride, 3-[2,6-bis(3,5-di-tert-butylphenyl)phenyl]-5-phenylthiazolium chloride, 3-(2,6-diphenylphenyl)-4,5,6,7-tetrahydrobenzothiazolium chloride, 3-[2,6-bis(3,5-di-tert-butylphenyl)phenyl]-4,5,6,7-tetrahydrobenzothiazolium chloride, 3-[2,6-bis(3,5-di-tert-butylphenyl)phenyl]-4,5,6,7-tetrahydro-7-methylbenzothiazolium chloride, 3-[2,6-bis(3,5-di-tert-butylphenyl)phenyl]-4,5,6,7-tetrahydro-7-ethylbenzothiazolium chloride, 3-[2,6-bis(3,5-di-tert-butylphenyl)phenyl]-4,5,6,7-tetrahydro-7-n-propylbenzothiazolium chloride, 3-[2,6-bis(3,5-di-tert-butylphenyl)phenyl]-4,5,6,7-tetrahydro-7-isopropylbenzothiazolium chloride, 3-[2,6-bis(3,5-di-tert-butylphenyl)phenyl]-4,5,6,7-tetrahydro-7-n-butylbenzothiazolium chloride, 3-[2,6-bis(3-tert-butylphenyl)phenyl]-4,5,6,7-tetrahydrobenzothiazolium chloride, 3-[2,6-bis(3,5-di-tert-butylphenyl)-4-methylphenyl]-4,5,6,7-tetrahydrobenzothiazolium chloride, 3-[2,6-bis(3,5-di-tert-butylphenyl)-4-chlorophenyl]-4,5,6,7-tetrahydrobenzothiazolium chloride, 3-[2,6-bis(3,5-di-tert-butylphenyl)-4-chlorophenyl]-4,5,6,7-tetrahydro-7-methylbenzothiazolium chloride, 3-[2,6-bis(3,5-di-tert-butylphenyl)-4-chlorophenyl]-4,5,6,7-tetrahydro-7-ethylbenzothiazolium chloride, 3-[2,6-bis(3,5-dibromophenyl)phenyl]-4,5,6,7-tetrahydrobenzothiazolium chloride, 3-[2,6-bis(3,5-dichlorophenyl)phenyl]-4,5,6,7-tetrahydrobenzothiazolium chloride, 3-[2,6-bis(3,5-difluorophenyl)phenyl]-4,5,6,7-tetrahydrobenzothiazolium chloride, 3-[2,6-bis(3,5-di-trifluoromethylphenyl)phenyl]-4,5,6,7-tetrahydrobenzothiazolium chloride, 3-[2,6-bis(3,5-dimethoxyphenyl)phenyl]-4,5,6,7-tetrahydrobenzothiazolium chloride, 3-[2,6-bis(3,5-di-methylphenyl)phenyl]-4,5,6,7-tetrahydrobenzothiazolium chloride, 5,6-dihydro-3-[2,6-bis(3,5-di-tert-butylphenyl)phenyl]-4H-cyclopentathiazolium chloride, 5,6,7,8-tetrahydro-3-[2,6-bis(3,5-di-tert-butylphenyl)phenyl]-4H-cycloheptathiazolium chloride, 3-(2,6-difluorophenyl)-4,5-dimethylthiazolium chloride, 3-(2,6-dichlorophenyl)-4,5-dimethylthiazolium chloride, 3-(2,6-dibromophenyl)-4,5-dimethylthiazolium chloride, 3-(2,6-dibromophenyl)-4-methyl-5-acylthiazolium chloride, 3-(2,6-diiodophenyl)-4,5-dimethylthiazolium chloride, 3-(2,6-dibromophenyl)-4,5,6,7-tetrahydrobenzothiazolium chloride, 3-(2,6-dibromophenyl)-4,5,6,7-tetrahydro-7-ethylbenzothiazolium chloride, 3-(2,4,6-tribromophenyl)-4,5,6,7-tetrahydrobenzothiazolium chloride, 3-(2-bromo-6-phenylphenyl)-4,5-dimethylthiazolium chloride, and 3-[2-chloro-6-(3,5-di-tert-butylphenyl)phenyl]-4,5-dimethylthiazolium chloride.

Examples may also include thiazolium salts (1′) obtained by substituting “chloride” in these thiazolium salts (1) with “iodide”, “bromide”, “methanesulfonate”, “trifluoromethanesulfonate”, “nitrate”, “perchlorate”, “tetrafluoroborate”, “tetrachloroborate”, “hexafluorophosphate”, “hexafluoroantimonate”, “hexachloroantimonate”, “pentafluorostannate”, “pentachlorostannate”, “tetraphenyl borate”, “tetrakis(pentafluorophenyl) borate”, and “tetrakis[3,5-bis(trifluoromethyl)phenyl] borate”.

Such a thiazolium salt (1′) is a novel compound and may be produced by a method disclosed in, for example, Scheme 1 in J. Amer. Chem. Soc., vol. 130, p. 2234 (2008). That is, a thiazolium salt (1′) can be produced by a method involving steps of N-formylation by causing a reaction of 2,6-diaryl-substituted aryl-1-amine, or 2,6-dihalo-substituted aryl-1-amine, or 2-halo-6-aryl-substituted aryl-1-amine with formic acid/acetic anhydride; thiocarbonylation by causing a reaction of the obtained N-formylated product with a Lawesson's reagent, phosphorus pentasulfide, or the like; and a reaction of the obtained thiocarbonylated product and a 2-halo-substituted-1-one compound.

Examples of a thiazolium salt (1″) may be 3-[2-(phenyl)-6-methylphenyl]-thiazolium chloride, 3-[2-(phenyl)-6-methylphenyl]-4,5-dimethylthiazolium chloride, 3-[2-(phenyl)-6-ethylphenyl]-4,5-diethylthiazolium chloride, 3-[2-(phenyl)-6-methylphenyl]-4-methyl-5-acylthiazolium chloride, 3-[2-(phenyl)-6-ethylphenyl]-4-methyl-5-methoxycarbonylthiazolium chloride, 3-[2-(3,5-di-tert-butylphenyl)-6-methylphenyl]-thiazolium chloride, 3-[2-(3,5-di-tert-butylphenyl)-6-methylphenyl]-4,5-dimethylthiazolium chloride, 3-[2-(3,5-di-tert-butylphenyl)-6-ethylphenyl]-4,5-diethylthiazolium chloride, 3-[2-(3,5-di-tert-butylphenyl)-6-methylphenyl]-4-methyl-5-acylthiazolium chloride, 3-[2-(3,5-di-tert-butylphenyl)-6-isopropylphenyl]-4-methyl-5-ethoxycarbonylthiazolium chloride, 3-[2-[3,5-di-trifluoromethylphenyl)-6-trifluoromethylphenyl]-4,5-dimethylthiazolium chloride, 3-[2-[3,5-di-trifluoromethylphenyl)-6-methylphenyl]-4,5-diethylthiazolium chloride, 3-[2-[3,5-di-methoxyphenyl)-6-methylphenyl]-4,5-dimethylthiazolium chloride, 3-[2-[3,5-di-methylphenyl)-6-methylphenyl]-4,5-dimethylthiazolium chloride, 3-[2-[3,5-di-methylphenyl)-6-ethylphenyl]-4,5-diethylthiazolium chloride, 3-[(2-phenyl-3,6-diisopropyl)phenyl]-4,5-dimethylthiazolium chloride, 3-[(2-phenyl-3,6-dimethyl)phenyl]-4,5-diethylthiazolium chloride, 3-[2-(3,5-dinitrophenyl)-6-methylphenyl]-4,5-dimethylthiazolium chloride, 3-[2-(3,5-dibromophenyl)-6-methylphenyl]-4,5-dimethylthiazolium chloride, 3-[(2-(3,5-dichlorophenyl)-3,6-diethyl)phenyl]-4,5-dimethylthiazolium chloride, 3-(2-phenyl-6-methylphenyl)-4,5,6,7-tetrahydrobenzothiazolium chloride, 3-[2-(3,5-di-tert-butylphenyl)-6-methylphenyl]-4,5,6,7-tetrahydrobenzothiazolium chloride, 3-[2-(3,5-di-tert-butylphenyl)-6-methylphenyl]-4,5,6,7-tetrahydro-7-methylbenzothiazolium chloride, 3-[2-(3,5-di-tert-butylphenyl)-6-methylphenyl]-4,5,6,7-tetrahydro-7-ethylbenzothiazolium chloride, 3-[2-(3,5-di-trifluoromethylphenyl)-6-methylphenyl]-4,5,6,7-tetrahydrobenzothiazolium chloride, 3-[2-(3,5-dimethoxyphenyl)-6-methylphenyl]-4,5,6,7-tetrahydrobenzothiazolium chloride, 3-[2-(3,5-di-methylphenyl)-6-ethylphenyl]-4,5,6,7-tetrahydrobenzothiazolium chloride, 3-(2-fluoro-6-methylphenyl)-4,5-dimethylthiazolium chloride, 3-(2-chloro-6-ethylphenyl)-4,5-dimethylthiazolium chloride, 3-(2-bromo-6-methylphenyl)-4,5-dimethylthiazolium chloride, 3-(2-bromo-6-methylphenyl)-4-methyl-5-acylthiazolium chloride, 3-(2-iodo-6-methylphenyl)-4,5-dimethylthiazolium chloride, 3-(2-bromo-6-methylphenyl)-4,5,6,7-tetrahydrobenzothiazolium chloride, 3-(2,4-dibromo-6-methylphenyl)-4,5,6,7-tetrahydrobenzothiazolium chloride, etc.

Examples may also include thiazolium salts (1″) obtained by substituting “chloride” in these thiazolium salts (1″) with “iodide”, “bromide”, “methanesulfonate”, “trifluoromethanesulfonate”, “nitrate”, “perchlorate”, “tetrafluoroborate”, “tetrachloroborate”, “hexafluorophosphate”, “hexafluoroantimonate”, “hexachloroantimonate”, “pentafluorostannate”, “pentachlorostannate”, “tetraphenyl borate”, “tetrakis(pentafluorophenyl) borate”, and “tetrakis[3,5-bis(trifluoromethyl)phenyl] borate”.

Such a thiazolium salt (1″) is a novel compound and may be produced by a method disclosed in, for example, Scheme 1 in J. Amer. Chem. Soc., vol. 130, p. 2234 (2008). That is, a thiazolium salt (1″) can be produced by a method involving steps of N-formylation by causing a reaction of 2-aryl-substituted aryl-1-amine or 2-halo-substituted aryl-1-amine with formic acid/acetic anhydride; thiocarbonylation by causing a reaction of the obtained N-formylated product with a Lawesson's reagent, phosphorus pentasulfide, or the like; and a reaction of the obtained thiocarbonylated product and a 2-halo-substituted-1-one compound.

Examples of a thiazolium salt (1) may be preferably 3-[2,6-di(phenyl)phenyl]-4,5-dimethylthiazolium salt, 3-[2,6-bis(3,5-di-tert-butylphenyl)phenyl]-4,5-dimethylthiazolium salt, 3-[(2,6-diphenyl-3,5-diisopropyl)phenyl]-4,5-dimethylthiazolium salt, 3-[2,6-bis(3,5-di-tert-butylphenyl)phenyl]-4,5,6,7-tetrahydrobenzothiazolium salt, 3-[2,6-bis(3,5-di-tert-butylphenyl)phenyl]-4,5,6,7-tetrahydro-7-ethylbenzothiazolium chloride, 3-[2,6-bis(3,5-di-tert-butylphenyl)-4-chlorophenyl]-4,5,6,7-tetrahydrobenzothiazolium salt, and 3-[2-(3,5-di-tert-butylphenyl)-6-methylphenyl]-4,5,6,7-tetrahydrobenzothiazolium salt.

A method for producing a compound as a thiazolium salt (1) in which W⁰ is an aryl, that is, a thiazolium salt defined by the above-mentioned formula (8) (hereinafter, referred to also as a thiazolium salt (8)) may be preferably a method involving a step A of causing a coupling reaction of a 3-(halo-substituted aryl)-2-thiazole-thione compound defined by the above-mentioned formula (6) (hereinafter, referred to also a 3-(halo-substituted aryl)-2-thiazole-thione compound (6)) and an aryl compound defined by the above-mentioned formula (5) (hereinafter, referred to also as an aryl compound (5)) in the presence of a palladium catalyst and a step B of oxidizing the 3-(aryl-substituted aryl)-2-thiazole-thione compound (hereinafter, referred to also as a 3-(aryl-substituted aryl)-2-thiazole-thione compound (7)) defined by the above-mentioned formula (7) and obtained in the step A.

As a method for producing a 3-(halo-substituted aryl)-2-thiazole-thione compound (6), there is a method involving steps of causing a reaction of a halo-substituted aryl-1-amine and carbon disulfide in the presence of a dimethyl sulfoxide solvent and sodium hydroxide and causing a reaction of the obtained product and a 2-halo-substituted-1-one compound.

Examples of a 3-(halo-substituted aryl)-2-thiazole-thione compound (6) may be 4,5-dimethyl-3-(2,6-difluorophenyl)-2(3H)-thiazole-thione, 4,5-dimethyl-3-(2,6-dichlorophenyl)-2(3H)-thiazole-thione, 4,5-dimethyl-3-(2,6-dibromophenyl)-2(3H)-thiazole-thione, 4,5-dimethyl-3-(2,6-diiodophenyl)-2(3H)-thiazole-thione, 4,5-diethyl-3-(2,6-dibromophenyl)-2(3H)-thiazole-thione, 3-(2,6-dibromophenyl)-2(3H)-thiazole-thione, 4,5-dimethyl-3-(2,4,6-tribromophenyl)-2(3H)-thiazole-thione, 4,5-dimethyl-3-(2,6-dibromo-4-chlorophenyl)-2(3H)-thiazole-thione, 4,5-dimethyl-3-(2,6-dibromo-4-methylphenyl)-2(3H)-thiazole-thione, 4,5-diethyl-3-(2,4,6-tribromophenyl)-2(3H)-thiazole-thione, 4-methyl-5-acyl-3-(2,6-dibromophenyl)-2(3H)-thiazole-thione, 4-methyl-5-methoxycarbonyl-3-(2,6-dibromophenyl)-2(3H)-thiazole-thione, 5-n-butyl-3-(2,6-dibromophenyl)-2(3H)-thiazole-thione, 5-tert-butyl-3-(2,6-dibromophenyl)-2(3H)-thiazole-thione, 5-phenyl-3-(2,6-dibromophenyl)-2(3H)-thiazole-thione, 4,5,6,7-tetrahydro-3-(2,6-dichlorophenyl)-2(3H)-benzothiazole-thione, 4,5,6,7-tetrahydro-3-(2,6-dibromophenyl)-2(3H)-benzothiazole-thione, 4,5,6,7-tetrahydro-7-methyl-3-(2,6-dibromophenyl)-2(3H)-benzothiazole-thione, 4,5,6,7-tetrahydro-7-ethyl-3-(2,6-dibromophenyl)-2(3H)-benzothiazole-thione, 4,5,6,7-tetrahydro-7-n-propyl-3-(2,6-dibromophenyl)-2(3H)-benzothiazole-thione, 4,5,6,7-tetrahydro-7-isopropyl-3-(2,6-dibromophenyl)-2(3H)-benzothiazole-thione, 4,5,6,7-tetrahydro-3-(2,4,6-tribromophenyl)-2(3H)-benzothiazole-thione, 4,5,6,7-tetrahydro-3-(2,6-dibromo-4-chlorophenyl)-2(3H)-benzothiazole-thione, 4,5,6,7-tetrahydro-3-(2,6-dibromo-4-methylphenyl)-2(3H)-benzothiazole-thione, 4,5,6,7-tetrahydro-3-(2,6-difluorophenyl)-2(3H)-benzothiazole-thione, 4,5,6,7-tetrahydro-3-(2,6-diiodophenyl)-2(3H)-benzothiazole-thione, 3,4,5,6-tetrahydro-3-(2,6-dibromophenyl)-2H-cyclopentathiazole-2-thione, 3,4,5,6,7-hexahydro-3-(2,6-dibromophenyl)-2H-cycloheptathiazole-2-thione, 3,4,5,6,7-hexahydro-3-(2,6-diiodophenyl)-2H-cycloheptathiazole-2-thione, 4,5-dimethyl-3-(2-fluorophenyl)-2(3H)-thiazole-thione, 4,5-dimethyl-3-(2-chlorophenyl)-2(3H)-thiazole-thione, 4,5-dimethyl-3-(2-bromophenyl)-2(3H)-thiazole-thione, 4,5-dimethyl-3-(2-iodophenyl)-2(3H)-thiazole-thione, 4,5-diethyl-3-(2-bromo-6-methylphenyl)-2(3H)-thiazole-thione, 3-(2-bromo-6-methylphenyl)-2(3H)-thiazole-thione, 4,5-dimethyl-3-(2,4-dibromo-6-ethylphenyl)-2(3H)-thiazole-thione, 4,5-diethyl-3-(2,4-dibromo-6-methylphenyl)-2(3H)-thiazole-thione, 4-methyl-5-acyl-3-(2-bromo-6-methylphenyl)-2(3H)-thiazole-thione, 4-methyl-5-methoxycarbonyl-3-(2-bromo-6-isopropylphenyl)-2(3H)-thiazole-thione, 4,5,6,7-tetrahydro-3-(2-chloro-6-methylphenyl)-2(3H)-benzothiazole-thione, 4,5,6,7-tetrahydro-3-(2-bromo-6-methylphenyl)-2(3H)-benzothiazole-thione, 4,5,6,7-tetrahydro-3-(2-bromophenyl)-2(3H)-benzothiazole-thione, 4,5,6,7-tetrahydro-3-(2,4-dibromophenyl)-2(3H)-benzothiazole-thione, 4,5,6,7-tetrahydro-3-(2,4-dibromo-6-methylphenyl)-2(3H)-benzothiazole-thione, 4,5,6,7-tetrahydro-3-(2-fluoro-6-methylphenyl)-2(3H)-benzothiazole-thione, 4,5,6,7-tetrahydro-3-(2-iodo-6-methylphenyl)-2(3H)-benzothiazole-thione, etc.

Next, the step A of carrying out a coupling reaction of a 3-(halo-substituted aryl)-2-thiazole-thione compound (6) and an aryl compound (5) in the presence of a palladium catalyst will be described.

An aryl compound which can give a 3-(aryl-substituted aryl)-2-thiazole-thione compound (7) by causing a coupling reaction of the carbon atom to which a leaving group denoted by L in the formula (5) is bonded and the carbon atom having a halogen atom as a substituent in the 3-(halo-substituted aryl)-2-thiazole-thione compound (6) may be used as the aryl compound (5) without any particular limit. Preferable examples of the leaving group denoted by L in the aryl compound (5) are —B(OH)₂ and —MgX⁰ (wherein, X⁰ denotes a halogen atom).

In the case the leaving group denoted by L is —B(OH)₂ (that is, the aryl compound (5) is arylboronic acid), the coupling reaction in the step A is preferably carried out in the presence of a base compound. Such a coupling reaction is so-called Suzuki-Miyaura coupling reaction and various improved methods have been developed and these methods may be used without any particular limit.

In the case the leaving group denoted by L is —MgX⁰ (that is, the aryl compound (5) is an aryl Grignard compound), the coupling reaction in the step A is preferably carried out in the presence of a zinc compound. Such a coupling reaction is so-called Negishi coupling reaction and various improved methods have been developed and these methods may be used without any particular limit.

Examples of the palladium catalyst to be used in the step A may be palladium atom-containing compounds without any particular limit in the valence of palladium atom or their ligands and preferably palladium catalysts such as tetrakis(triphenylphosphine) palladium complex consisting of palladium atom and a ligand containing phosphorus atom. The palladium catalyst may be commercialized products or those prepared by causing a reaction of a phosphorus compound and a palladium compound.

Examples of a palladium compound to be used for preparing the palladium catalyst may be 1,5-diphenyl-1,4-pentadien-3-one (palladium) complex, bis(1,5-diphenyl-1,4-pentadien-3-one) (palladium) complex, tris(1,5-diphenyl-1,4-pentadien-3-one)di(palladium)chloroform complex, allylpalladium chloride dimer, cyclooctadienepalladium dichloride, cyclooctadienepalladium dibromide, norbornadienepalladium dibromide, acetic acid palladium, palladiumacetylacetone, bis(acetonitrile)dichloro palladium, and bis(benzonitrile)dichloro palladium. These palladium compounds may be used alone or two or more kinds may be used in the form of a mixture.

One kind of phosphorus compound or two or more kinds of phosphorus compounds in the form of a mixture may be used as the phosphorus compound for preparing the palladium catalyst. The phosphorus compound is a compound having one or more trivalent phosphorus atoms in a molecule and examples may be phosphorus compounds defined by

### PR⁹R¹⁰R¹¹

(wherein R⁹, R¹⁰ and R¹¹ independently denote an alkyl which may have a substituent, an aryl which may have a substituent, an alkoxy which may have a substituent, or an aryloxy which may have a substituent).

Examples of an alkyl denoted independently by R⁰, R¹⁰ and R¹¹ may be C₁₋₂₀ straight chain, branched chain, and cyclic alkyl such as methyl, ethyl, n-propyl, isopropyl, n-butyl, isobutyl, sec-butyl, tert-butyl, n-pentyl, n-decyl, cyclopropyl, cyclopentyl, cyclohexyl, and menthyl. These alkyl groups may have at least one kind of group selected from the group consisting of alkoxy such as methoxy and ethoxy; a halogen atom such as a fluorine atom, a chlorine atom, and a bromine atom; alkoxycarbonyl such as methoxycarboxyl and ethoxycarbonyl; aryl such as phenyl, 1-naphthyl, and 2-naphthyl; and carboxy and examples of alkyl having such a group may be chloromethyl, fluoromethyl, trifluoromethyl, methoxymethyl, ethoxymethyl, 2-methoxyethyl, methoxycarbonylmethyl, and benzyl. Examples of an aryl denoted independently by R⁹, R¹⁰ and R¹¹ may be C₆₋₁₀ aryl such as phenyl, 1-naphthyl, 2-naphthyl, and ferrocenyl. The aryl groups may have the above-mentioned alkyl, aryl, alkoxy, halogen atom and examples of an aryl which may have these groups may be phenyl, 1-naphthyl, 2-naphthyl, 2-methylphenyl, 4-chlorophenyl, 4-methylphenyl, and 4-methoxyphenyl.

Examples of an alkoxy denoted independently by R⁹, R¹⁰ and R¹¹ may be C₁₋₂₀ straight chain, branched chain, and cyclic alkoxy such as methoxy, ethoxy, n-propoxy, isopropoxy, n-butoxy, isobutoxy, sec-butoxy, tert-butoxy, n-pentyloxy, n-decyloxy, cyclopropoxy, cyclopentyloxy, and cyclohexyloxy. These alkoxy groups may have at least one kind of group selected from the group consisting of alkoxy such as methoxy and ethoxy; a halogen atom such as a fluorine atom, a chlorine atom, and a bromine atom; alkoxycarbonyl such as methoxycarbonyl and ethoxycarbonyl; aryl such as phenyl and naphthyl; and carboxy and examples of alkoxy having such a group may be chloromethoxy, fluoromethoxy, trifluoromethoxy, methoxymethoxy, ethoxymethoxy, 2-methoxyethoxy, and benzyloxy.

Examples of an aryloxy denoted independently by R⁹, R¹⁰ and R¹¹ may be C₆₋₁₀ aryloxy such as phenoxy and naphthyloxy. These aryloxy groups may have a substituent such as the above-mentioned alkyl, aryl, alkoxy, and halogen atom. Examples of these aryloxy groups may be phenoxy, 1-naphthyloxy, 2-naphthyloxy, 2-methylphenoxy, 4-chlorophenoxy, 4-methylphenoxy, and 4-methoxyphenoxy.

An alkyl, an aryl, an alkoxy, and an aryloxy denoted independently by R⁹, R¹⁰ and R¹¹ may have a group defined by —PR⁹R¹⁰ (wherein R⁹ and R¹⁰ independently denote as described above).

Examples of such a phosphorus compound may be triphenylphosphine, tris(4-chlorophenyl)phosphine, tris(4-methoxyphenyl)phosphine, (2-di-tert-butylphosphino)biphenyl, bis(diphenylphosphino)ethane, bis(diphenylphosphino)propane, bis(diphenylphosphino)butane, 1,1′-bis(diphenylphosphino)ferrocene, 2,2′-bis(diphenylphosphino)-1,1′-binaphthalene, 2,2′-bis(diphenylphosphino)-1,1′-biphenyl, 1,1′-oxybis[2,1-phenylenebis(diphenylphosphine)], triisopropylphosphine, tri(tert-butyl)phosphine, tricyclohexylphosphine, triisopropylphosphite, tricyclohexylphosphite, and triphenylphosphite and preferably (2-di-tert-butylphosphino) biphenyl.

The reaction of a phosphorus compound and a palladium compound may be carried out by mixing the phosphorus compound and the palladium compound in the presence of a solvent before the reaction in the step A or may be carried out in the reaction system in the presence of a 3-(halo-substituted aryl)-2-thiazole-thione compound (6), which is a raw material of the reaction in the step A, a base compound and also an arylboronic acid.

The use amount of the palladium compound is preferably in a range of 0.000001 mol to 0.2 mol and more preferably in a range of 0.0001 mol to 0.1 mol to 1 mol of the 3-(halo-substituted aryl)-2-thiazole-thione compound (6). The use amount of the phosphorus compound is preferably in a range of 1 mol to 10 mol and more preferably in a range of 1 mol to 3 mol on the basis of phosphorus atom to 1 mol of palladium atom contained in the palladium compound.

Examples of an aryl compound (5) having a leaving group —B(OH)₂ denoted by L; that is, an arylboronic acid; may be phenylboronic acid, 3,5-difluorophenylboronic acid, 1-naphthylboronic acid, 3,5-dinitrophenylboronic acid, 3,5-bis(trifluoromethyl)phenylboronic acid, 3,5-dimethylphenylboronic acid, 3,5-dimethoxyphenylboronic acid, 3,5-di-tert-butylphenylboronic acid, 3,5-diphenylphenylboronic acid, etc.

The use amount of the arylboronic acid is for example in a range of 1 mol to 10 mol and preferably in a range of 1 mol to 5 mol to 1 mol of the 3-(halo-substituted aryl)-2-thiazole-thione compound (6).

A base compound to be used together with the arylboronic acid in the step A may be those which do not inhibit the reaction and can neutralize boronic acid without any particular limit and examples may be alkali metal fluorides such as potassium fluoride, cesium fluoride, and rubidium fluoride; and alkali metal carbonates and alkaline earth metal carbonates such as sodium carbonate, potassium carbonate, calcium carbonate, and magnesium carbonate.

The use amount of the base compound is preferably in a range of 1 mol to 5 mol and more preferably in a range of 1 mol to 3 mol to 1 mol of the arylboronic acid.

Examples of an aryl compound (5) having a leaving group —MgX⁰ denoted by L; that is, an aryl Grignard compound; may be phenylmagnesium bromide, 3,5-difluorophenylmagnesium bromide, 1-naphthylmagnesium bromide, 3,5-bis(trifluoromethyl)phenylmagnesium bromide, 3,5-dimethylphenylmagnesium bromide, 3,5-dimethoxyphenylmagnesium bromide, 3,5-di-tert-butylphenylmagnesium bromide, 3,5-diphenylphenylmagnesium bromide, phenylmagnesium chloride, 3,5-difluorophenylmagnesium chloride, 1-naphthylmagnesium chloride, 3,5-bis(trifluoromethyl)phenylmagnesium chloride, 3,5-dimethylphenylmagnesium chloride, 3,5-dimethoxyphenylmagnesium chloride, 3,5-di-tert-butylphenylmagnesium chloride, 3,5-diphenylphenylmagnesium chloride, phenylmagnesium iodide, 3,5-difluorophenylmagnesium iodide, 1-naphthylmagnesium iodide, 3,5-bis(trifluoromethyl)phenylmagnesium iodide, 3,5-dimethylphenylmagnesium iodide, 3,5-dimethoxyphenylmagnesium iodide, 3,5-di-tert-butylphenylmagnesium iodide, 3,5-diphenylphenylmagnesium iodide, etc.

Commercialized products and those which are produced by a conventional method of preparing a Grignard reagent from a corresponding aryl halide and magnesium metal may be used as the aryl Grignard compound.

The use amount of the aryl Grignard compound is for example in a range of 1 mol to 10 mol and preferably in a range of 1 mol to 5 mol to 1 mol of the 3-(halo-substituted aryl)-2-thiazole-thione compound (6).

Examples of a zinc compound to be used together with the aryl Grignard compound in the step A may be a zinc halide such as zinc chloride and zinc bromide.

The use amount of the zinc compound is preferably in a range of 0.5 mol to 3 mol and more preferably in a range of 0.8 mol to 2 mol to 1 mol of the aryl Grignard compound.

The reaction in the step A is generally carried out in the presence of a solvent. A solvent to be used in the case an arylboronic acid is used as the aryl compound (5) is preferably an organic solvent or a mixed solvent of an organic solvent and water. Examples of the organic solvent may be an ether solvent such as diethyl ether, methyl tert-butyl ether, and tetrahydrofuran; a halo hydrocarbon solvent such as chloroform and chlorobenzene; an aromatic solvent such as toluene and xylene; an alcohol solvent such as methanol, ethanol, isopropanol, and tert-butanol; and a nitrile solvent such as acetonitrile and propionitrile. A solvent to be used in the case an aryl Grignard compound is used as the aryl compound (5) is preferably an organic solvent. Examples of the organic solvent may be an ether solvent such as diethyl ether, methyl tert-butyl ether, and tetrahydrofuran; a halo hydrocarbon solvent such as chloroform and chlorobenzene; and an aromatic solvent such as toluene and xylene.

The use amount of the solvent is not particularly limited and in consideration of the volume efficiency, it may be practically at highest 100 times as much as a (3-halo-substituted aryl)-2-thiazole-thione compound (6) on the basis of weight.

The reaction in the step A may be carried out in normal pressure or in a pressurized condition.

The reaction temperature is preferably in a range of −20° C. to 150° C. and more preferably in a range of 0° C. to 100° C. If the reaction temperature is higher than 150° C., a byproduct having a high boiling point tends to be increased by a side reaction and if the reaction temperature is lower than −20° C., the reactivity tends to be lowered.

In the case an arylboronic acid is used as an aryl compound (5), the reaction in the step A is preferably carried out by mixing a 3-(halo-substituted aryl)-2-thiazole-thione compound (6), the arylboronic acid, a palladium catalyst, a base compound, and if necessary, a solvent, and mixing and stirring the mixture at a prescribed temperature. The mixing order is not particularly limited. In the case an aryl Grignard compound is used as an aryl compound (5), the reaction is preferably carried out by mixing the aryl Grignard compound, a palladium catalyst, a zinc compound, and if necessary, a solvent, thereafter adding a 3-(halo-substituted aryl)-2-thiazole-thione compound (6), and mixing and stirring the mixture at a prescribed temperature.

The progression degree of the reaction may be confirmed by an analysis means such as gas chromatography, high performance liquid chromatography, thin-layer chromatography, nuclear magnetic resonance spectroscopic analysis, infrared absorption spectrometry, etc.

The obtained reaction mixture generally contains a 3-(aryl-substituted aryl)-2-thiazole-thione compound (7) and on completion of the reaction, the 3-(aryl-substituted aryl)-2-thiazole-thione compound (7) is separated and taken out by concentration treatment or precipitation treatment after the palladium catalyst is removed by filtration or using a short column if necessary.

The obtained 3-(aryl-substituted aryl)-2-thiazole-thione compound (7) may be refined further by a refining means such as recrystallization, column chromatography, etc.

The 3-(aryl-substituted aryl)-2-thiazole-thione compound (7) is preferably the above-mentioned 3-(aryl-substituted aryl)-2-thiazole-thione compound (7′) and the above-mentioned 3-(aryl-substituted aryl)-2-thiazole-thione compound (7″).

In the above-mentioned formula (7′), it is preferable that R¹ and R² are independently a C₁₋₁₀ (more preferably a C₁₋₄) alkyl, or that R¹ and R² are bonded to each other to form a C₅₋₇ cycloalkene ring (more preferably a cyclohexene ring) together with the carbon atoms bonded to R¹ and R²; it is more preferable that the carbon atom of the cycloalkene ring in the thiazolium ring S side (the 7th position in the cyclohexene ring) has a C₁₋₄ alkyl as a substituent; it is preferable that R³, R⁴, and R⁵ are independently a hydrogen atom, a halogen atom, or a C₁₋₁₀ alkyl; and it is preferable that W¹ and W² are independently a C₆₋₁₀ aryl which may have a substituent. The aryl is more preferably phenyl having a substituent, even more preferably phenyl having a bulky group at one of the 3rd and the 5th positions, and still more preferably phenyl having a bulky group at the 3rd and the 5th positions. Examples of the bulky group may be phenyl, trifluoromethyl, tert-butyl, a chlorine atom, a bromine atom, an iodine atom, nitro, cyano, methoxycarbonyl, acyl, sulfo, and 3,4,5-trifluorophenyl.

In the above-mentioned formula (7″), it is preferable that R¹ and R² are independently a C₁₋₁₀ (more preferably a C₁₋₄) alkyl, or that R¹ and R² are bonded to each other to form a C₅₋₇ cycloalkene ring (more preferably a cyclohexene ring) together with the carbon atoms bonded to R¹ and R²; it is more preferable that the carbon atom of the cycloalkene ring in the thiazolium ring S side (the 7th position in the cyclohexene ring) has a C₁₋₄ alkyl as a substituent; it is preferable that R³, R⁴, and R⁵ are independently a hydrogen atom or a C₁₋₁₀ alkyl; it is preferable that R⁸ is a C₁₋₁₀ alkyl; and it is preferable that W² is a C₆₋₁₀ aryl which may have a substituent. The aryl is more preferably phenyl having a substituent, even more preferably phenyl having a bulky group at one of the 3rd and the 5th positions, and still more preferably phenyl having a bulky group at the 3rd and the 5th positions. Examples of the bulky group may be phenyl, trifluoromethyl, tert-butyl, a chlorine atom, a bromine atom, an iodine atom, nitro, cyano, methoxycarbonyl, acyl, sulfo, and 3,4,5-trifluorophenyl.

Examples of a 3-(aryl-substituted aryl)-2-thiazole-thione compound (7) may be 4,5-dimethyl-3-[2,6-bis(3,5-di-tert-butylphenyl)phenyl]-2(3H)-thiazole-thione, 4,5-dimethyl-3-[2,6-bis(3,5-di-tert-butylphenyl)-4-chlorophenyl]-2(3H)-thiazole-thione, 4,5-dimethyl-3-[2,6-bis(3,5-di-tert-butylphenyl)-4-methylphenyl]-2(3H)-thiazole-thione, 4,5-dimethyl-3-[2,6-bis(3,5-dichlorophenyl)phenyl]-2(3H)-thiazole-thione, 4,5-dimethyl-3-[2,6-bis(3,5-dibromophenyl)phenyl]-2(3H)-thiazole-thione, 4,5-dimethyl-3-[2,6-bis(3-tert-butylphenyl)-4-chlorophenyl]-2(3H)-thiazole-thione, 4,5-dimethyl-3-(2,6-diphenylphenyl)-2(3H)-thiazole-thione, 4,5-dimethyl-3-[2,6-bis(3,5-di-trifluoromethylphenyl)phenyl])-2(3H)-thiazole-thione, 4,5-dimethyl-3-[2,6-bis(3,5-dimethoxyphenyl)phenyl]-2(3H)-thiazole-thione, 4,5-diethyl-3-[2,6-bis(3,5-dimethylphenyl)phenyl]-2(3H)-thiazole-thione, 3-[2,6-bis(3,5-di-tert-butylphenyl)phenyl]-2(3H)-thiazole-thione, 4,5-diethyl-3-[2,6-bis(3,5-di-tert-butylphenyl)phenyl]-2(3H)-thiazole-thione, 4-methyl-5-acyl-3-[2,6-bis(3,5-di-tert-butylphenyl)phenyl]-2(3H)-thiazole-thione, 4-methyl-5-methoxycarbonyl-3-[2,6-bis(3,5-di-tert-butylphenyl)phenyl]-2(3H)-thiazole-thione, 5-n-butyl-3-[2,6-bis(3,5-di-tert-butylphenyl)phenyl]-2(3H)-thiazole-thione, 5-tert-butyl-3-[2,6-bis(3,5-di-tert-butylphenyl)phenyl]-2(3H)-thiazole-thione, 5-phenyl-3-[2,6-bis(3,5-di-tert-butylphenyl)phenyl]-2(3H)-thiazole-thione, 4,5,6,7-tetrahydro-3-[2,6-bis(3,5-di-tert-butylphenyl)phenyl]-2(3H)-benzothiazole-thione, 4,5,6,7-tetrahydro-7-methyl-3-[2,6-bis(3,5-di-tert-butylphenyl)phenyl]-2(3H)-benzothiazole-thione, 4,5,6,7-tetrahydro-7-ethyl-3-[2,6-bis(3,5-di-tert-butylphenyl)phenyl]-2(3H)-benzothiazole-thione, 4,5,6,7-tetrahydro-7-n-propyl-3-[2,6-bis(3,5-di-tert-butylphenyl)phenyl]-2(3H)-benzothiazole-thione, 4,5,6,7-tetrahydro-7-isopropyl-3-[2,6-bis(3,5-di-tert-butylphenyl)phenyl]-2(3H)-benzothiazole-thione, 4,5,6,7-tetrahydro-7-n-butyl-3-[2,6-bis(3,5-di-tert-butylphenyl)phenyl]-2(3H)-benzothiazole-thione, 4,5,6,7-tetrahydro-3-[2,6-bis(3,5-di-tert-butylphenyl)-4-methylphenyl]-2(3H)-benzothiazole-thione, 4,5,6,7-tetrahydro-3-[2,6-bis(3,5-di-tert-butylphenyl)-4-chlorophenyl]-2(3H)-benzothiazole-thione, 4,5,6,7-tetrahydro-7-ethyl-3-[2,6-bis(3,5-di-tert-butylphenyl)-4-chlorophenyl]-2(3H)-benzothiazole-thione, 4,5,6,7-tetrahydro-3-[2,6-bis(3,5-dichlorophenyl)phenyl]-2(3H)-benzothiazole-thione, 4,5,6,7-tetrahydro-3-[2,6-bis(3,5-dibromophenyl)phenyl]-2(3H)-benzothiazole-thione, 4,5,6,7-tetrahydro-3-[2,6-bis(3,5-dimethylphenyl)phenyl]-2(3H)-benzothiazole-thione, 4,5,6,7-tetrahydro-3-[2,6-bis(3,5-ditrifluoromethylphenyl)phenyl]-2(3H)-benzothiazole-thione, 4,5,6,7-tetrahydro-3-[2,6-bis(3,5-dimethoxyphenyl)phenyl]-2(3H)-benzothiazole-thione, 3,4,5,6-tetrahydro-3-[2,6-bis(3,5-di-tert-butylphenyl)phenyl]-2H-cyclopentathiazole-2-thione, 3,4,5,6,7-hexahydro-3-[2,6-bis(3,5-di-tert-butylphenyl)phenyl]-2H-cycloheptathiazole-2-thione, 4,5,6,7-tetrahydro-3-[2-chloro-6-(3,5-di-tert-butylphenyl)phenyl]-2(3H)-benzothiazole-thione, 4,5,6,7-tetrahydro-3-[2-bromo-6-(3,5-di-tert-butylphenyl)phenyl]-2(3H)-benzothiazole-thione, 4,5-dimethyl-3-[2-bromo-6-(3,5-di-tert-butylphenyl)phenyl]-2(3H)-thiazole-thione, 4,5-dimethyl-3-(2-bromo-6-phenyl)phenyl-2(3H)-thiazole-thione, 4,5-dimethyl-3-[2-(3,5-di-tert-butylphenyl)phenyl]-2(3H)-thiazole-thione, 4,5-dimethyl-3-[2-(3,5-di-tert-butylphenyl)-6-methylphenyl]-2(3H)-thiazole-thione, 4,5-dimethyl-3-(2-phenylphenyl)-2(3H)-thiazole-thione, 4,5-dimethyl-3-(2-phenyl-6-isopropylphenyl)-2(3H)-thiazolone, 4,5-dimethyl-3-[2-(3,5-di-trifluoromethylphenyl)-6-methylphenyl])-2(3H)-thiazole-thione, 4,5-dimethyl-3-[2-(3,5-dimethoxyphenyl)-6-ethylphenyl]-2(3H)-thiazole-thione, 4,5-diethyl-3-[2-(3,5-dimethylphenyl)-6-methylphenyl]-2(3H)-thiazole-thione, 3-[2-(3,5-di-tert-butylphenyl)-6-methylphenyl]-2(3H)-thiazole-thione, 4,5-diethyl-3-[2-(3,5-di-tert-butylphenyl)phenyl]-2(3H)-thiazole-thione, 4-methyl-5-acyl-3-[2-(3,5-di-tert-butylphenyl)-6-methylphenyl]-2(3H)-thiazole-thione, 4-methyl-5-methoxycarbonyl-3-[2-(3,5-di-tert-butylphenyl)-6-ethylphenyl]-2(3H)-thiazole-thione, 4,5,6,7-tetrahydro-3-[2-(3,5-di-tert-butylphenyl)phenyl]-2(3H)-benzothiazole-thione, 4,5,6,7-tetrahydro-3-[2-(3,5-di-tert-butylphenyl)-6-methylphenyl]-2(3H)-benzothiazole-thione, 4,5,6,7-tetrahydro-7-methyl-3-[2-(3,5-di-tert-butylphenyl)-6-methylphenyl]-2(3H)-benzothiazole-thione, 4,5,6,7-tetrahydro-7-ethyl-3-[2-(3,5-di-tert-butylphenyl)-6-methylphenyl]-2(3H)-benzothiazole-thione, 4,5,6,7-tetrahydro-3-[2-(3,5-dimethylphenyl)-6-methylphenyl]-2(3H)-benzothiazole-thione, 4,5,6,7-tetrahydro-3-[2-(3,5-ditrifluoromethylphenyl)-6-trifluoromethylphenyl]-2(3H)-benzothiazole-thione, 4,5,6,7-tetrahydro-3-[2-(3,5-dimethoxyphenyl)-6-methylphenyl]-2(3H)-benzothiazole-thione, etc.

Next, the step B of oxidizing the 3-(aryl-substituted aryl)-2-thiazole-thione compound (7) obtained in the step A will be described.

The oxidation in the step B is generally carried out by mixing the 3-(aryl-substituted aryl)-2-thiazole-thione compound (7) and an oxidizing agent. Examples of the oxidizing agent may be hydrogen peroxide, hypochlorous acid, and perbenzoic acid and preferably hydrogen peroxide. An aqueous solution of 10 wt. % to 60 wt. % of hydrogen peroxide is preferably used.

The use amount of the oxidizing agent is preferably in a range of 2 mol to 20 mol and more preferably in a range of 2 mol to 10 mol to 1 mol of the 3-(aryl-substituted aryl)-2-thiazole-thione compound (7).

The reaction in the step B is generally carried out in the presence of a solvent. A solvent to be used is an organic solvent or a mixed solvent of an organic solvent and water. Examples of the organic solvent may be a halo hydrocarbon solvent such as chloroform and chlorobenzene; an aromatic solvent such as toluene and xylene; a carboxylic acid solvent such as acetic acid and trifluoroacetic acid; and a nitrile solvent such as acetonitrile and propionitrile. The carboxylic acid solvent is preferable. The use amount of the organic solvent is not particularly limited and in consideration of the volume efficiency, it may be practically at highest 100 times as much as a (3-aryl-substituted aryl)-2-thiazole-thione compound (7) on the basis of weight.

The reaction in the step B may be carried out in normal pressure or in a pressurized condition.

The reaction temperature is preferably in a range of 0° C. to 150° C. and more preferably in a range of 20° C. to 100° C. If the reaction temperature is higher than 150° C., a byproduct having a high boiling point tends to be increased by a side reaction and if the reaction temperature is lower than 0° C., the reactivity tends to be lowered.

The reaction in the step B is carried out by mixing and stirring the 3-(aryl-substituted aryl)-2-thiazole-thione compound (7), an oxidizing agent, and if necessary, a solvent at a desired temperature. The mixing order is not particularly limited.

The progression degree of the reaction may be confirmed by an analysis means such as gas chromatography, high performance liquid chromatography, thin-layer chromatography, nuclear magnetic resonance spectroscopic analysis, infrared absorption spectrometry, etc.

On completion of the reaction, after the remaining oxidizing agent is removed by reduction treatment using sodium thiosulfate, concentration treatment, or extraction with a solvent in which a thiazolium salt (8) is dissolved and washing with water for removal in a water layer if necessary, the produced thiazolium salt (8) is separated and taken out by forming a desired anion defined by X⁻ by salt exchange operation and carrying out crystallization treatment.

The salt exchange operation can be carried out by, for example, exchanging X⁻ to chloride ion by carrying out extraction in an organic solvent such as a halo hydrocarbon solvent, an ether solvent, or the like in which the thiazolium salt (8) can be dissolved, mixing and stirring the resulting solvent with an aqueous saturated solution of sodium chloride, magnesium chloride, or the like, and separating liquid phases; thereafter removing the solvent, or dissolving the thiazolium salt (8) in an alcohol solvent; and then precipitating the thiazolium salt (8) in the form of a perchloric acid salt or a tetrafluoroboric acid salt by using lithium perchlorate or lithium tetrafluoroborate.

A thiazolium salt (1) in which W⁰′ is a halogen atom can also be obtained by carrying out oxidation in the step B using a 3-(halo-substituted aryl)-2-thiazole-thione compound (6) in place of a 3-(aryl-substituted aryl)-2-thiazole-thione compound (7).

The present invention is characterized by carrying out a coupling reaction (that is, the present reaction) of an aldehyde compound in the presence of a thiazolium salt (1) and a base compound. The present reaction is advantageous in terms of improvement of selectivity in production of an α-hydroxy ketone compound per unit catalyst amount.

The present invention is characterized by carrying out a coupling reaction (that is, the present reaction) of an aldehyde compound in the presence of a thiazol-2-ylidene defined by a formula (1-2) (hereinafter, sometimes referred to as a thiazol-2-ylidene (1-2)).

Generally, the reaction of a thiazolium salt (1) and a base compound produces a thiazol-2-ylidene (1-2) which can work as a catalyst for the coupling reaction of an aldehyde compound. A thiazol-2-ylidene (1-2) may have stability changed in accordance with its structure and those which are stabilized by a bulky substituent in a carbene position can be observed by NMR and IR. The present reaction can be performed by mixing the thiazolium salt (1), a base compound, and an aldehyde compound with no need of confirming production of the thiazol-2-ylidene (1-2) and also can be performed by producing the thiazol-2-ylidene (1-2) from only the thiazolium salt (1) and a base compound and then adding an aldehyde compound.

The structure of the thiazol-2-ylidene (1-2) is formed by pulling out hydrogen in the form of proton from the carbon atom at the 2nd position of the thiazolium ring of the thiazolium salt (1) by the basic group and neutralizing the carbon atom together with X⁻ anion to produce carbene in the carbon atom at the 2nd position.

The thiazol-2-ylidene (1-2) is preferably a thiazol-2-ylidene defined by a formula (1′-2) (hereinafter, referred to also as a thiazol-2-ylidene (1′-2)) and a thiazol-2-ylidene defined by a formula (1″-2) (hereinafter, referred to also as a thiazol-2-ylidene (1″-2)).

Examples of a thiazol-2-ylidene (1′-2) are 3-[2,6-di(phenyl)phenyl]-thiazol-2-ylidene, 3-[2,6-di(phenyl)phenyl]-4,5-dimethylthiazol-2-ylidene, 3-[2,6-di(phenyl)phenyl]-4,5-diethylthiazol-2-ylidene, 3-[2,6-di(phenyl)phenyl]-4-methyl-5-acylthiazol-2-ylidene, 3-[2,6-di(phenyl)phenyl]-4-methyl-5-methoxycarbonylthiazol-2-ylidene, 3-[2,6-bis(3,5-di-tert-butylphenyl)phenyl]-thiazol-2-ylidene, 3-[2,6-bis(3,5-di-tert-butylphenyl)phenyl]-4,5-dimethylthiazol-2-ylidene, 3-[2,6-bis(3-tert-butylphenyl)phenyl]-4,5-dimethylthiazol-2-ylidene, 3-[2,6-bis(3,5-di-tert-butylphenyl)-4-methylphenyl]-4,5-dimethylthiazol-2-ylidene, 3-[2,6-bis(3,5-di-tert-butylphenyl)-4-chlorophenyl]-4,5-dimethylthiazol-2-ylidene, 3-[2,6-bis(3,5-dibromophenyl)phenyl]-4,5-dimethylthiazol-2-ylidene, 3-[2,6-bis(3,5-dichlorophenyl)phenyl]-4,5-dimethylthiazol-2-ylidene, 3-[2,6-bis(3,5-di-tert-butylphenyl)phenyl]-4,5-diethylthiazol-2-ylidene, 3-[2,6-bis(3,5-di-tert-butylphenyl)phenyl]-4-methyl-5-acylthiazol-2-ylidene, 3-[2,6-bis(3,5-di-tert-butylphenyl)phenyl]-4-methyl-5-ethoxycarbonylthiazol-2-ylidene, 3-[2,6-bis(3,5-di-trifluoromethylphenyl)phenyl]-4,5-dimethylthiazol-2-ylidene, 3-[2,6-bis(3,5-di-trifluoromethylphenyl)phenyl]-4,5-diethylthiazol-2-ylidene, 3-[2,6-bis(3,5-di-methoxyphenyl)phenyl]-4,5-dimethylthiazol-2-ylidene, 3-[2,6-bis(3,5-di-methoxyphenyl)phenyl]-4,5-diethylthiazol-2-ylidene, 3-[2,6-bis(3,5-di-methylphenyl)phenyl]-4,5-dimethylthiazol-2-ylidene, 3-[2,6-bis(3,5-di-methylphenyl)phenyl]-4,5-diethylthiazol-2-ylidene, 3-[(2,6-diphenyl-3,5-diisopropyl)phenyl]-thiazol-2-ylidene, 3-[(2,6-diphenyl-3,5-diisopropyl)phenyl]-4,5-dimethylthiazol-2-ylidene, 3-[(2,6-diphenyl-3,5-diisopropyl)phenyl]-4,5-diethylthiazol-2-ylidene, 3-[2,6-bis(3,5-dinitrophenyl)phenyl]-4,5-dimethylthiazol-2-ylidene, 3-[(2,6-diphenyl-3,5-dimethyl)phenyl]-4,5-dimethylthiazol-2-ylidene, 3-[2,6-bis(3,5-dibromophenyl)phenyl]-4,5-dimethylthiazol-2-ylidene, 3-[(2,6-bis-(3,5-dichlorophenyl)-3,5-diisopropyl)phenyl]-4,5-dimethylthiazol-2-ylidene, 3-[2,6-bis(3,5-di-tert-butylphenyl)phenyl]-5-n-butylthiazol-2-ylidene, 3-[2,6-bis(3,5-di-tert-butylphenyl)phenyl]-5-tert-butylthiazol-2-ylidene, 3-[2,6-bis(3,5-di-tert-butylphenyl)phenyl]-5-phenylthiazol-2-ylidene, 3-(2,6-diphenylphenyl)-4,5,6,7-tetrahydrobenzothiazol-2-ylidene, 3-[2,6-bis(3,5-di-tert-butylphenyl)phenyl]-4,5,6,7-tetrahydrobenzothiazol-2-ylidene, 3-[2,6-bis(3,5-di-tert-butylphenyl)phenyl]-4,5,6,7-tetrahydro-7-methylbenzothiazol-2-ylidene, 3-[2,6-bis(3,5-di-tert-butylphenyl)phenyl]-4,5,6,7-tetrahydro-7-ethylbenzothiazol-2-ylidene, 3-[2,6-bis(3,5-di-tert-butylphenyl)phenyl]-4,5,6,7-tetrahydro-7-n-propylbenzothiazol-2-ylidene, 3-[2,6-bis(3,5-di-tert-butylphenyl)phenyl]-4,5,6,7-tetrahydro-7-isopropylbenzothiazol-2-ylidene, 3-[2,6-bis(3,5-di-tert-butylphenyl)phenyl]-4,5,6,7-tetrahydro-7-n-butylbenzothiazol-2-ylidene, 3-[2,6-bis(3-tert-butylphenyl)phenyl]-4,5,6,7-tetrahydrobenzothiazol-2-ylidene, 3-[2,6-bis(3,5-di-tert-butylphenyl)-4-methylphenyl]-4,5,6,7-tetrahydrobenzothiazol-2-ylidene, 3-[2,6-bis(3,5-di-tert-butylphenyl)-4-chlorophenyl]-4,5,6,7-tetrahydrobenzothiazol-2-ylidene, 3-[2,6-bis(3,5-di-tert-butylphenyl)-4-chlorophenyl]-4,5,6,7-tetrahydro-7-methylbenzothiazol-2-ylidene, 3-[2,6-bis(3,5-di-tert-butylphenyl)-4-chlorophenyl]-4,5,6,7-tetrahydro-7-ethylbenzothiazol-2-ylidene, 3-[2,6-bis(3,5-dibromophenyl)phenyl]-4,5,6,7-tetrahydrobenzothiazol-2-ylidene, 3-[2,6-bis(3,5-dichlorophenyl)phenyl]-4,5,6,7-tetrahydrobenzothiazol-2-ylidene, 3-[2,6-bis(3,5-difluorophenyl)phenyl]-4,5,6,7-tetrahydrobenzothiazol-2-ylidene, 3-[2,6-bis(3,5-di-trifluoromethylphenyl)phenyl]-4,5,6,7-tetrahydrobenzothiazol-2-ylidene, 3-[2,6-bis(3,5-dimethoxyphenyl)phenyl]-4,5,6,7-tetrahydrobenzothiazol-2-ylidene, 3-[2,6-bis(3,5-di-methylphenyl)phenyl]-4,5,6,7-tetrahydrobenzothiazol-2-ylidene, 5,6-dihydro-3-[2,6-bis(3,5-di-tert-butylphenyl)phenyl]-4H-cyclopentathiazol-2-ylidene, 5,6,7,8-tetrahydro-3-[2,6-bis(3,5-di-tert-butylphenyl)phenyl]-4H-cycloheptathiazol-2-ylidene, 3-(2,6-difluorophenyl)-4,5-dimethylthiazol-2-ylidene, 3-(2,6-dichlorophenyl)-4,5-dimethylthiazol-2-ylidene, 3-(2,6-dibromophenyl)-4,5-dimethylthiazol-2-ylidene, 3-(2,6-dibromophenyl)-4-methyl-5-acylthiazol-2-ylidene, 3-(2,6-diiodophenyl)-4,5-dimethylthiazol-2-ylidene, 3-(2,6-dibromophenyl)-4,5,6,7-tetrahydrobenzothiazol-2-ylidene, 3-(2,6-dibromophenyl)-4,5,6,7-tetrahydro-7-ethylbenzothiazol-2-ylidene, 3-(2,4,6-tribromophenyl)-4,5,6,7-tetrahydrobenzothiazol-2-ylidene, 3-(2-bromo-6-phenylphenyl)-4,5-dimethylthiazol-2-ylidene, 3-[2-chloro-6-(3,5-di-tert-butylphenyl)phenyl]-4,5-dimethylthiazol-2-ylidene, etc.

Examples of a thiazol-2-ylidene (1″-2) are 3-[2-(phenyl)-6-methylphenyl]-thiazol-2-ylidene, 3-[2-(phenyl)-6-methylphenyl]-4,5-dimethylthiazol-2-ylidene, 3-[2-(phenyl)-6-ethylphenyl]-4,5-diethylthiazol-2-ylidene, 3-[2-(phenyl)-6-methylphenyl]-4-methyl-5-acylthiazol-2-ylidene, 3-[2-(phenyl)-6-ethylphenyl]-4-methyl-5-methoxycarbonylthiazol-2-ylidene, 3-[2-(3,5-di-tert-butylphenyl)-6-methylphenyl]-thiazol-2-ylidene, 3-[2-(3,5-di-tert-butylphenyl)-6-methylphenyl]-4,5-dimethylthiazol-2-ylidene, 3-[2-(3,5-di-tert-butylphenyl)-6-ethylphenyl]-4,5-diethylthiazol-2-ylidene, 3-[2-(3,5-di-tert-butylphenyl)-6-methylphenyl]-4-methyl-5-acylthiazol-2-ylidene, 3-[2-(3,5-di-tert-butylphenyl)-6-isopropylphenyl]-4-methyl-5-ethoxycarbonylthiazol-2-ylidene, 3-[2-[3,5-di-trifluoromethylphenyl)-6-trifluoromethylphenyl]-4,5-dimethylthiazol-2-ylidene, 3-[2-[3,5-di-trifluoromethylphenyl)-6-methylphenyl]-4,5-diethylthiazol-2-ylidene, 3-[2-[3,5-di-methoxyphenyl)-6-methylphenyl]-4,5-dimethylthiazol-2-ylidene, 3-[2-[3,5-di-methylphenyl)-6-methylphenyl]-4,5-dimethylthiazol-2-ylidene, 3-[2-[3,5-di-methylphenyl)-6-ethylphenyl]-4,5-diethylthiazol-2-ylidene, 3-[(2-phenyl-3,6-diisopropyl)phenyl]-4,5-dimethylthiazol-2-ylidene, 3-[(2-phenyl-3,6-dimethyl)phenyl]-4,5-diethylthiazol-2-ylidene, 3-[2-(3,5-dinitrophenyl)-6-methylphenyl]-4,5-dimethylthiazol-2-ylidene, 3-[2-(3,5-dibromophenyl)-6-methylphenyl]-4,5-dimethylthiazol-2-ylidene, 3-[(2-(3,5-dichlorophenyl)-3,6-diethyl)phenyl]-4,5-dimethylthiazol-2-ylidene, 3-(2-phenyl-6-methylphenyl)-4,5,6,7-tetrahydrobenzothiazol-2-ylidene, 3-[2-(3,5-di-tert-butylphenyl)-6-methylphenyl]-4,5,6,7-tetrahydrobenzothiazol-2-ylidene, 3-[2-(3,5-di-tert-butylphenyl)-6-methylphenyl]-4,5,6,7-tetrahydro-7-methylbenzothiazol-2-ylidene, 3-[2-(3,5-di-tert-butylphenyl)-6-methylphenyl]-4,5,6,7-tetrahydro-7-ethylbenzothiazol-2-ylidene, 3-[2-(3,5-di-trifluoromethylphenyl)-6-methylphenyl]-4,5,6,7-tetrahydrobenzothiazol-2-ylidene, 3-[2-(3,5-dimethoxyphenyl)-6-methylphenyl]-4,5,6,7-tetrahydrobenzothiazol-2-ylidene, 3-[2-(3,5-di-methylphenyl)-6-ethylphenyl]-4,5,6,7-tetrahydrobenzothiazol-2-ylidene, 3-(2-fluoro-6-methylphenyl)-4,5-dimethylthiazol-2-ylidene, 3-(2-chloro-6-ethylphenyl)-4,5-dimethylthiazol-2-ylidene, 3-(2-bromo-6-methylphenyl)-4,5-dimethylthiazol-2-ylidene, 3-(2-bromo-6-methylphenyl)-4-methyl-5-acylthiazol-2-ylidene, 3-(2-iodo-6-methylphenyl)-4,5-dimethylthiazol-2-ylidene, 3-(2-bromo-6-methylphenyl)-4,5,6,7-tetrahydrobenzothiazol-2-ylidene, 3-(2,4-dibromo-6-methylphenyl)-4,5,6,7-tetrahydrobenzothiazol-2-ylidene, etc.

Such thiazol-2-ylidenes (1′-2) and (1″-2) are novel compounds and as described in the present invention, a thiazolium salt (1) and a base compound can be reacted. As another production method, the method described in JP 5-221913 A can be employed for synthesizing these compounds. That is, thiazol-2-ylidenes (1′-2) and (1″-2) can be produced by a method of heating a precursor obtained by adding an alcohol or carbon dioxide to the carbon atom at the 2nd position of a thiazol-2-ylidene.

Thiazol-2-ylidenes (1′-2) and (1″-2) are preferably 3-[2,6-di(phenyl)phenyl]-4,5-dimethylthiazol-2-ylidene, 3-[2,6-bis(3,5-di-tert-butylphenyl)phenyl]-4,5-dimethylthiazol-2-ylidene, 3-[(2,6-diphenyl-3,5-diisopropyl)phenyl]-4,5-dimethylthiazol-2-ylidene, 3-[2,6-bis(3,5-di-tert-butylphenyl)phenyl]-4,5,6,7-tetrahydrobenzothiazol-2-ylidene, 3-[2,6-bis(3,5-di-tert-butylphenyl)phenyl]-4,5,6,7-tetrahydro-7-ethylbenzothiazol-2-ylidene, 3-[2,6-bis(3,5-di-tert-butylphenyl)-4-chlorophenyl]-4,5,6,7-tetrahydrobenzothiazol-2-ylidene, or 3-[2-(3,5-di-tert-butylphenyl)-6-methylphenyl]-4,5,6,7-tetrahydrobenzothiazol-2-ylidene.

An aldehyde compound to be used is not particularly limited if it is a compound having at least one formyl in its molecule. The coupling reaction in the present invention may include a homo-coupling reaction for coupling a single aldehyde compound and a cross-coupling reaction for coupling different aldehyde compounds.

The homo-coupling reaction may be a homo-coupling reaction of an aldehyde compound defined by a formula (2)

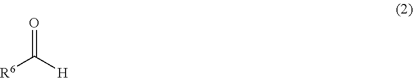

(wherein R⁶ denotes a hydrogen atom, an alkyl which may have a substituent, an aryl which may have a substituent, or a heteroaryl which may have a substituent)(hereinafter, referred to also as an aldehyde (2)).

The homo-coupling reaction of an aldehyde (2) gives an α-hydroxy ketone compound (hereinafter, referred to also as an α-hydroxy ketone (3)) defined by a formula (3)

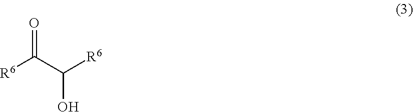

(wherein R⁶ denotes the same as described above).

The cross-coupling reaction may be a cross-coupling reaction of an aldehyde (2) and an aldehyde compound defined by a formula (4)

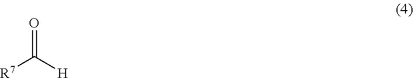

(wherein R⁷ is different from R⁶ and denotes a hydrogen atom, an alkyl which may have a substituent, an aryl which may have a substituent, or a heteroaryl which may have a substituent) (hereinafter, referred to also as an aldehyde (4)).

The cross-coupling reaction of an aldehyde (2) and an aldehyde (4) gives an α-hydroxy ketone compound defined by a formula (5)

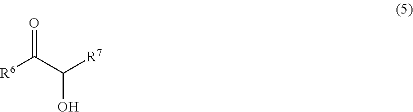

(wherein R⁶ and R⁷ denotes the same as described above), an α-hydroxy ketone compound defined by a formula (6)

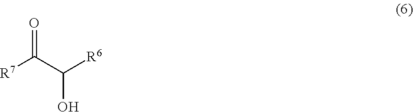

(wherein R⁶ and R⁷ denotes the same as described above), or their mixture. The production ratio differs in accordance with the types of R⁵ and R⁶ and one of them may be produced selectively in some cases.

Examples of an alkyl denoted by R⁶ and R⁷ may be C₁₋₁₀ straight chain, branched chain, and cyclic alkyl such as methyl, ethyl, propyl, isopropyl, butyl, isobutyl, sec-butyl, tert-butyl, pentyl, decyl, cyclopropyl, 2,2-dimethylcyclopropyl, cyclopentyl, cyclohexyl, and menthyl.

Examples of a substituent which the alkyl may have may be a C₁₋₆ alkoxy, which may have a fluorine atom, such as methoxy, ethoxy, propoxy, isopropoxy, butoxy, isobutoxy, sec-butoxy, tert-butoxy, and trifluoromethyloxy; a C₆₋₁₀-aryl-containing C₁₋₁₀ alkoxy, which may have a C₁₋₁₀ alkoxy, such as benzyloxy, 4-methylbenzyloxy, and 4-methoxybenzyloxy; a C₁₋₁₀ alkoxy having a C₆₋₁₀ aryloxy-containing C₆₋₁₀ aryl such as 3-phenoxybenzyloxy; a C₆₋₁₀ aryloxy, which may have a C₁₋₁₀ alkoxy, such as phenoxy, 2-methylphenoxy, 4-methylphenoxy, and 4-methoxyphenoxy; a C₆₋₁₀ aryloxy-containing C₆₋₁₀ aryloxy such as 3-phenoxyphenoxy; a C₂₋₁₀ acyl, which may have a C₁₋₁₀ alkoxy, such as acetyl, propionyl, benzylcarbonyl, 4-methylbenzylcarbonyl, 4-methoxybenzylcarbonyl, benzoyl, 2-methylbenzoyl, 4-methylbenzoyl, and 4-methoxybenzoyl; a C₁₋₁₀ alkylthio such as methylthio, ethylthio, and isopropylthio; a C₂₋₁₀ alkoxycarbonyl such as methoxycarbonyl and ethoxycarbonyl; and a halogen atom such as a fluorine atom, a chlorine atom, and a bromine atom.

Examples of an alkyl having a substituent may be chloromethyl, fluoromethyl, trifluoromethyl, methoxymethyl, ethoxymethyl, methoxyethyl, methoxycarbonylmethyl, 1-ethoxycarbonyl-2,2-dimethyl-3-cyclopropyl, and 2-methylthioethyl.

Examples of an aryl denoted by R⁶ and R₇ may be a C₆₋₂₀ aryl such as phenyl, 2-methylphenyl, 4-methylphenyl, and naphthyl.

Examples of a substituent which the aryl may have may be a C₁₋₁₀ alkyl containing a fluorine atom such as fluoromethyl and trifluoromethyl; a C₁₋₁₀ alkyl containing a C₁₋₁₀ alkoxy such as methoxymethyl, ethoxymethyl, and methoxyethyl; a C₁₋₁₀ alkoxy which may have a fluorine atom or a C₁₋₁₀ alkoxy such as methoxy, ethoxy, propoxy, isopropoxy, butoxy, isobutoxy, sec-butoxy, tert-butoxy, pentyloxy, cyclopentyloxy, fluoromethoxy, trifluoromethoxy, methoxymethoxy, ethoxymethoxy, and methoxyethoxy; a C₆₋₁₀ aryloxy which may have a C₁₋₁₀ alkoxy such as phenoxy, 2-methylphenoxy, 4-methylphenoxy, and 4-methoxyphenoxy; a C₆₋₁₀ aryloxy which may have a C₆₋₁₀ aryloxy such as 3-phenoxyphenoxy; a C₂₋₁₀ acyl which may have a C₁₋₁₀ alkoxy such as acetyl, propionyl, benzylcarbonyl, 4-methylbenzylcarbonyl, and 4-methoxybenzylcarbonyl; a nitro group; a halogen atom such as a fluorine atom and a chlorine atom; and a C₁₋₆ alkylenedioxy such as methylenedioxy. Examples of an aryl having a substituent may be 4-chlorophenyl, 4-methoxyphenyl, and 3-phenoxyphenyl.

Examples of a heteroaryl in R⁶ and R⁷ may be a C₄₋₁₀ heteroaryl containing at least one hetero atom such as a nitrogen atom, an oxygen atom, and a sulfur atom, such as pyridyl, furyl, and 5-methylfuryl.

Examples of a substituent which the heteroaryl may have may be a C₁₋₁₀ alkyl containing a fluorine atom such as fluoromethyl and trifluoromethyl; a C₁₋₁₀ alkyl containing a C₁₋₁₀ alkoxy such as methoxymethyl, ethoxymethyl, and methoxyethyl; a C₁₋₁₀ alkoxy which may have a fluorine atom or a C₁₋₁₀ alkoxy such as methoxy, ethoxy, propoxy, isopropoxy, butoxy, isobutoxy, sec-butoxy, tert-butoxy, pentyloxy, cyclopentyloxy, fluoromethoxy, trifluoromethoxy, methoxymethoxy, ethoxymethoxy, and methoxyethoxy; a C₆₋₁₀ aryloxy which may have a C₁₋₁₀ alkoxy such as phenoxy, 2-methylphenoxy, 4-methylphenoxy, and 4-methoxyphenoxy; a C₆₋₁₀ n aryloxy which may have a C₆₋₁₀ aryloxy such as 3-phenoxyphenoxy; a C₂₋₁₀ acyl which may have a C₁₋₁₀ alkoxy such as acetyl, propionyl, benzylcarbonyl, 4-methylbenzylcarbonyl, and 4-methoxybenzylcarbonyl; a nitro group; and a halogen atom such as a fluorine atom and a chlorine atom.

Examples of a heteroaryl having a substituent may include 2-chloropyridyl.

Examples of an aldehyde (2) and an aldehyde (4) may be an aliphatic aldehyde such as formaldehyde, acetaldehyde, propionaldehyde, n-butyl aldehyde, cyclopentane carboaldehyde, cyclohexane carboaldehyde, 2-methylpropanal, 2,2-dimethylpropanal, 3-methylthiopropanal, 2,2-dimethylbutanal, 1-methylcyclohexane carboaldehyde, 2,2-dimethylnonanal, and methyl 2,2-dimethyl-3-oxopropanate; an aromatic aldehyde such as benzaldehyde, 4-fluorobenzaldehyde, 4-nitrobenzaldehyde, 3-bromobenzaldehyde, 2-chlorobenzaldehyde, 4-methylbenzaldehyde, 3-methoxybenzaldehyde, 3,4,5-trimethoxybenzaldehyde, 3,4-methylenedioxybenzaldehyde, and 1-naphthoaldehyde; and a hetero-aromatic aldehyde such as picoline aldehyde and nicotine aldehyde. As an aldehyde (2) and an aldehyde (4), polymers of formaldehyde such as paraformaldehyde may also be usable and an aldehyde may be used in a state of co-existing with water, e.g., formalin water. In the case of a cross-coupling reaction of an aldehyde (2) and an aldehyde (4), the aldehyde (2) and the aldehyde (4) are different from each other. It is preferable to use formaldehyde co-existing with water as the aldehyde (4) and it is more preferable to use 3-methylthiopropanal as the aldehyde (2).

Commercialized ones and those which are produced by a conventional method may be used as an aldehyde compound.

The present reaction is preferable to be caused in the presence of a solvent.

Examples of the solvent may be an aromatic hydrocarbon solvent such as toluene, xylene, and chlorobenzene; an aliphatic hydrocarbon solvent such as pentane, hexane, and heptane; a halo hydrocarbon solvent such as dichloromethane, dichloroethane, and chloroform; an ether solvent such as diethyl ether, methyl tert-butyl ether, and tetrahydrofuran; an ester solvent such as ethyl acetate; an amide solvent such as N,N-dimethylformamide and N,N-dimethylacetamide; and an alcohol solvent such as methanol and ethanol.

In the case an aqueous solution such as formalin water; that is, formaldehyde co-existing with water; is used as an aldehyde compound, the reaction can be caused efficiently by using a solvent which has no compatibility with water. Examples to be used as a solvent which has no compatibility with water may be preferably the above-mentioned aromatic hydrocarbon solvents; aliphatic hydrocarbon solvents; and halo hydrocarbon solvents.

The use amount of the solvent may be, in consideration of the volume efficiency, practically preferably 100 parts by weight or less to 1 part by weight of an aldehyde compound.

In the case the present reaction is a homo-coupling reaction, the use amount of a thiazolium salt (1) is preferably 0.00001 to 0.2 mol and more preferably 0.0001 to 0.05 mol to 1 mol of an aldehyde compound.

In the case the present reaction is a cross-coupling reaction, the use amount of a thiazolium salt (1) is preferably 0.00001 to 0.2 mol and more preferably 0.0001 to 0.05 mol to 1 mol of one of aldehyde compounds. In the case the present reaction is a cross-coupling reaction, generally, 1 mol or more of one aldehyde compound is used to 1 mol of the other aldehyde compound.

A base compound to be used in the present reaction may be at least one kind of compound selected from the group consisting of organic bases, alkali metal salts such as alkali metal carbonates, alkaline earth metal salts such as alkaline earth metal carbonates.

Examples of an organic base may be a tertiary amine such as triethylamine, trioctylamine, diisopropylethylamine, and 4-dimethylaminopyridine; a nitrogen-containing cyclic compound such as 1,8-diazabicyclo[5.4.0]-7-undecene, 1,5,7-triazabicyclo[4,4,0]-5-decene; a nitrogen-containing aromatic compound such as pyridine and imidazole; and an alkali metal alkoxide such as sodium methoxide and sodium ethoxide.

Examples of an alkali metal carbonate may be sodium carbonate, sodium hydrogen carbonate, potassium carbonate, potassium hydrogen carbonate, lithium carbonate, and lithium hydrogen carbonate.

Examples of an alkaline earth metal carbonate may be magnesium carbonate and calcium carbonate.

A base compound is preferably an organic base.

The use amount of the base compound is preferably in a range of 0.1 to 2 mol and more preferably in a range of 0.5 to 1.5 mol to 1 mol of the thiazolium salt (1).

The present reaction may be carried out in the presence of carbon dioxide or in a gas atmosphere inactive to the reaction without using carbon dioxide. Carbon dioxide to be used in the present reaction may be gaseous or solid-phase (dry ice) or in supercritical state.

Gaseous carbon dioxide may be diluted with an inert gas such as nitrogen.

The use amount of carbon dioxide is preferably 1 mol or more to 1 mol in total of base compounds and its upper limit is not particularly limited and in consideration of the productivity, it may be 1000 mol or lower. Examples of the gas inactive to the reaction may be nitrogen, argon, and helium and its use amount is not particularly limited.

The reaction temperature of the present reaction may be in a range of −20° C. to 200° C.

The present reaction is caused by, for example, a method of mixing an aldehyde compound, a thiazolium salt (1), a base compound, and if necessary, a solvent. The mixing order is not particularly limited and a method to be employed preferably may be a method of mixing an aldehyde compound, a thiazolium salt (1) and if necessary a solvent and thereafter adding a base compound or a method of mixing a thiazolium salt (1), a base compound, and if necessary a solvent, and thereafter adding an aldehyde compound to the mixture. A method in which the present reaction is carried out in carbon dioxide or in an inactive gas atmosphere is employed more preferably.

The present reaction may be carried out in normal pressure or in a pressurized condition, for example, by pressurizing with gaseous carbon dioxide or a gas inactive to the reaction.

The progression degree of the reaction may be confirmed by an analysis means such as gas chromatography, high performance liquid chromatography, thin-layer chromatography, NMR, IR, etc.

An α-hydroxy ketone compound can be taken out, for example, by concentrating the obtained reaction mixture on completion of the present reaction. The taken out α-hydroxy ketone compound may be refined further by a refining means such as distillation, column chromatography, etc.

Examples of an α-hydroxy ketone compound to be obtained in the above-mentioned manner may be 2-hydroxyacetaldehyde, 3-hydroxy-2-butanone, 4-hydroxy-3-hexanone, 1,6-dimethylthio-4-hydroxy-3-hexanone, 5-hydroxy-4-octanone, 2-hydroxy-1-phenyl-ethanone, 2-hydroxy-1-(4-chlorophenyl)-ethanone, 2-hydroxy-1-phenyl-2-phenylethanone, 2-hydroxy-1-(4-methoxyphenyl)-2-phenylethanone, 2-hydroxy-1-(4-chlorophenyl)-2-phenylethanone, 2-hydroxy-1-(2-fluorophenyl)-2-phenylethanone, 4-(methylthio)-2-oxo-1-butanol, 1-hydroxy-2-propanone, 1-hydroxy-2-butanone, 1-hydroxy-2-pentanone, and 2-hydroxy-1-cyclohexanone.

### EXAMPLES

Hereinafter, the present invention will be described in detail with reference to Examples.

### Example 1

### Synthesis of 3-[2,6-di(phenyl)phenyl]-4,5-dimethylthiazolium chloride

A 300 mL flask purged with nitrogen was loaded with 6.6 g of 2,6-diphenylformanilide, 150 g of toluene, and 7.34 g of a Lawesson's reagent. The obtained mixture was heated to 60° C. and kept warm and stirred for 2 hours. After cooled to room temperature, the obtained reaction mixture was filtered and the obtained crystal was washed with 10 g of toluene and thereafter the filtrate and the washing solution were mixed and concentrated to obtain 5.3 g of a yellow solid material.

After a 50 mL flask purged with nitrogen was loaded with the entire amount of the obtained yellow solid material, 8 g of dioxane, and 2.6 g of 3-chloro-2-butanone, the mixture was heated and refluxed at a bath temperature of 110° C. for 5 hours. The reaction solution was cooled to room temperature and mixed with 33 g of water and 1 g of sodium carbonate and then concentrated to obtain 8.1 g of a dark brown oil. This oil was mixed with 150 mL of chloroform, the precipitated crystal was removed by filtration and washed with 10 g of chloroform, and the filtrate and the washing solution were mixed and concentrated to obtain 4.3 g of a brown oil. This oil was fractionated by a silica gel column (chloroform:ethanol=85:15) and an eluted portion with high polarity was concentrated to obtain 0.88 g of a light brown crystal.

The obtained crystal was confirmed to be 3-[2,6-di(phenyl)phenyl]-4,5-dimethylthiazolium chloride by ¹H-NMR. Yield: 9.6%

¹H-NMR (δ/ppm, CDCL3, on the basis of tetramethylsilane): 1.90 (s, 3H), 2.25 (s, 3H), 7.2-7.8 (m, 13H), 11.66 (s, 1H)

### Example 2

### Synthesis of 3-[2,6-bis(3,6-di-tert-butylphenyl)phenyl]-4,5-dimethylthiazolium chloride

A 100 mL flask purged with nitrogen was loaded with 0.9 g of formic acid and 0.82 g of acetic anhydride and the mixture was stirred at room temperature for 1 hour and then cooled to 5° C. A solution obtained by dissolving 3.03 g of 2,6-bis(3,5-di-tert-butylphenyl)aniline in 15 g of chloroform was dropwise added to the resulting reaction solution at 5° C. for 30 minutes. Thereafter, the obtained reaction solution was kept warm and stirred at room temperature for 3 hours. After concentrated, the obtained reaction mixture was mixed with 20 g of chloroform, neutralized and washed with 15 g of a saturated sodium hydrogen carbonate solution and washed with 15 g of water and then the formed chloroform layer was dried and concentrated by anhydrous sodium sulfate to obtain 3.2 g of a white solid material. The obtained white solid material was confirmed to be 2,6-bis(3,5-di-tert-butylphenyl)formanilide by gas chromatography/mass spectroscopy (GC-MS). Yield 98%, M⁺=497

A 300 mL flask purged with nitrogen was loaded with 0.5 g of 2,6-bis(3,5-di-tert-butylphenyl)formanilide, 5 g of toluene, and 0.31 g of a Lawesson's reagent. The obtained mixture was heated to 60° C. and kept warm and stirred for 2 hours. After cooled to room temperature, the obtained reaction mixture was filtered and the obtained crystal was washed with 10 g of toluene and thereafter, the filtrate and the washing solution were mixed and concentrated to obtain 0.38 g of a yellow solid material.

After a 50 mL flask purged with nitrogen was loaded with the entire amount of the obtained yellow solid material, 1 g of dioxane, and 0.11 g of 3-chloro-2-butanone, the mixture was heated and refluxed at a bath temperature of 110° C. for 18 hours. The reaction solution was cooled to room temperature to obtain 0.48 g of a brown oil. This oil was fractionated by a silica gel column (chloroform:ethanol=85:15) and an eluted portion with high polarity was concentrated to obtain 0.10 g of a light brown crystal.

The obtained crystal was confirmed to be 3-[2,6-bis(3,5-di-tert-butylphenyl)phenyl]-4,5-dimethylthiazolium chloride by ¹H-NMR.

Yield: 16.5%

¹H-NMR (δ/ppm, CDCL3, on the basis of tetramethylsilane): 1.24 (s, 36H), 1.90 (s, 3H), 2.22 (s, 3H), 7.2-7.65 (m, 9H), 11.0 (s, 1H)

### Example 3

### Synthesis of 3-[(2,6-diphenyl-3,5-diisopropyl)phenyl]-4,5-dimethylthiazolium chloride

A 100 mL flask purged with nitrogen was loaded with 2.6 g of 2,6-diphenyl-3,5-diisopropyliodobenzene, 70 g of hexane, and 15 g of ethanol and the mixture was cooled to −78° C. by an acetone-dry ice bath. After 7.3 mL of a n-butyllithium/hexane solution (1.63 M) was added for 10 minutes to the obtained solution, the solution mixture was heated to room temperature and kept at the temperature and stirred for 7 hours. After the reaction solution was cooled to 0° C. and 10 g of a tosyl azide/toluene solution with 11 to 15 wt. % was added to the resulting reaction solution for 5 minutes, the reaction solution was heated to room temperature and kept at the temperature and stirred for 12 hours. After mixed with 80 g of water, the obtained reaction mixture was subjected to extraction with 80 g of diethyl ether and the formed organic layer was washed with water, dried by anhydrous magnesium sulfate, mixed with 15 g of a molecular sieve (MS-3A), and dried overnight.

A 500 mL flask purged with nitrogen was loaded with the entire amount of the solution obtained by removing MS-3A and magnesium sulfate from the above-mentioned dried solution by filtration and a solution obtained by suspending 0.38 g of lithium aluminum hydride in 50 mL of diethyl ether was added to the solution at room temperature for 20 minutes and the resulting solution mixture was refluxed and stirred further for 4 hours. After the reaction, the reaction solution mixture was cooled to room temperature and quenched by adding 2 g of water little by little and then mixed with 0.5 g of an aqueous solution of 15 wt. % of sodium hydroxide and 1.2 g of water and stirred for 15 minutes. After a precipitated white crystal was removed by filtration, an organic layer obtained by adding 400 mL of diethyl ether and 200 mL of water and carrying out extraction and fractionation was dried with magnesium sulfate and concentrated to obtain 2.1 g of an orange color crystal. The obtained crystal was refined by a silica gel short column (hexane/ethyl acetate=10:1) to obtain 0.74 g of an orange color crystal. The obtained orange color crystal was confirmed to be (2,6-diphenyl-3,5-diisopropyl)aniline by GC-MS. Yield 39%, M⁺=329

A 100 mL flask purged with nitrogen was loaded with 0.31 g of formic acid and 0.29 g of acetic anhydride and the mixture was stirred at room temperature for 1 hour and then cooled to 5° C. A solution obtained by dissolving 0.74 g of (2,6-diphenyl-3,5-diisopropyl)aniline in 5 g of chloroform was dropwise added to the resulting reaction solution at 5° C. for 30 minutes. Thereafter, the obtained reaction solution was kept warm and stirred at room temperature for 3 hours. The precipitated crystal was filtered from the obtained reaction mixture, washed with water, and dried to obtain 0.4 g of a white crystal. After concentrated, the filtrate was mixed with 20 g of chloroform, neutralized and washed with 15 g of a saturated sodium hydrogen carbonate solution and washed with 15 g of water and then the formed chloroform layer was dried and concentrated by anhydrous sodium sulfate to obtain 0.2 g of a white solid material. The obtained two kinds of white crystals were confirmed to be both (2,6-diphenyl-3,5-diisopropyl)formanilide by GC-MS. Yield 74%, M⁺=357

A 30 mL flask purged with nitrogen was loaded with 0.6 g of (2,6-diphenyl-3,5-diisopropyl)formanilide, 50 g of toluene, and 0.5 g of a Lawesson's reagent. The obtained mixture was heated to 60° C. and kept warm and stirred for 3 hours. After cooled to room temperature, the obtained reaction mixture was filtered and the obtained crystal was washed with 10 g of toluene and thereafter, the filtrate and the washing solution were mixed and concentrated to obtain 0.97 g of a yellow solid material.

After a 50 mL flask purged with nitrogen was loaded with the entire amount of the obtained yellow solid material, 3 g of dioxane, and 0.18 g of 3-chloro-2-butanone, the mixture was heated and refluxed at a bath temperature of 110° C. for 3 hours. The reaction solution was cooled to room temperature to obtain 1.34 g of a brown oil. This oil was fractionated by a silica gel column (chloroform:ethanol=85:15) and an eluted portion with high polarity was concentrated to obtain 0.24 g of a light brown crystal. The obtained crystal was confirmed to be 3-[(2,6-diphenyl-3.5-diisopropyl)phenyl]-4,5-dimethylthiazolium chloride by ¹H-NMR.

Yield: 34%

¹H-NMR (δ/ppm, CDCL3, on the basis of tetramethylsilane): 1.0-1.3 (m, 12H), 1.85 (s, 3H), 2.05 (s, 3H), 2.7 (m, 2H), 6.9-7.6 (m, 11H), 11.1 (s, 1H)

### Example 4

### Synthesis of 4,5-dimethyl-3-(2,6-dibromophenyl)-2(3H)-thiazole-thione

A 100 mL flask purged with nitrogen was loaded with 10 g of 2,6-dibromoaniline and 30 g of dimethyl sulfoxide and the mixture was cooled to 5° C. while being stirred. The flask was further loaded with a solution obtained by dissolving 1.6 g of a sodium hydroxide powder in 1.5 g of water and the mixture was stirred for 10 minutes. Further, 3.0 g of carbon disulfide was added for 10 minutes and the mixture was heated to room temperature and stirred for 1 hour and cooled to 5° C. The resulting reaction solution was mixed with 4.3 g of 3-chloro-2-butanone, heated to room temperature, and stirred for 1 hour. When the resulting reaction solution was mixed with 40 g of water and stirred for 30 minutes, the reaction solution was separated into a gum-like solid and a water layer and therefore, the water layer was removed by decantation and the gum-like solid was washed with 40 g of water. This solid was mixed with 50 g of ethanol and 5 g of concentrated hydrochloric acid and heated and stirred at 70° C. for 30 minutes. After the reaction, the reaction mixture was cooled to room temperature, and the precipitated crystal was filtered and successively washed with 5 g of ethanol and dried to obtain 4.5 g of a light brown powder. This crystal was confirmed to be 4,5-dimethyl-3-(2,6-dibromophenyl)-2(3H)-thiazole-thione by GC-MS. Yield 30%, M⁺=379

### Example 5

### Synthesis of 4,5,6,7-tetrahydro-3-(2,6-dichlorophenyl)-2(3H)-benzothiazole-thione

A 100 mL flask purged with nitrogen was loaded with 5 g of 2,6-dichloroaniline and 10 g of dimethyl sulfoxide and the mixture was cooled to 5° C. while being stirred. The flask was further loaded with a solution obtained by dissolving 1.3 g of a sodium hydroxide powder in 1.2 g of water and the mixture was stirred for 10 minutes. Further, 2.4 g of carbon disulfide was added for 10 minutes and the mixture was heated to room temperature and stirred for 1 hour and cooled to 5° C. The resulting reaction solution was mixed with 4.1 g of 2-chlorocyclohexanone, heated to room temperature, and stirred for 1 hour. When the resulting reaction solution was mixed with 30 g of water and stirred for 30 minutes, the reaction solution was separated into a gum-like solid and a water layer and therefore, the water layer was removed by decantation and the gum-like solid was washed with 30 g of water. This solid was mixed with 50 g of ethanol and 5 g of concentrated hydrochloric acid and heated and stirred at 70° C. for 30 minutes. After the reaction, the reaction mixture was cooled to room temperature, and the precipitated crystal was filtered and successively washed with 5 g of ethanol and dried to obtain 3.1 g of a light yellow powder. This crystal was confirmed to be 4,5,6,7-tetrahydro-3-(2,6-dichlorophenyl)-2(3H)-benzothiazole-thione by GC-MS. Yield 32%, M⁺=316

### Example 6

### Synthesis of 4,5,6,7-tetrahydro-3-(2,6-dibromophenyl)-2(3H)-benzothiazole-thione

A 100 mL flask purged with nitrogen was loaded with 2 g of 2,6-dibromoaniline and 6 g of dimethyl sulfoxide and the mixture was cooled to 5° C. while being stirred. The flask was further loaded with a solution obtained by dissolving 320 mg of a sodium hydroxide powder in 300 mg of water and the mixture was stirred for 10 minutes. Further, 610 mg of carbon disulfide was added for 10 minutes and the mixture was heated to room temperature and stirred for 1 hour and cooled to 5° C. The resulting reaction solution was mixed with 1.06 g of 2-chlorocyclohexanone, heated to room temperature, and stirred for 1 hour. When the resulting reaction solution was mixed with 10 g of water and stirred for 30 minutes, the reaction solution was separated into a gum-like solid and a water layer and therefore, the water layer was removed by decantation and the gum-like solid was washed with 10 g of water. This solid was mixed with 10 g of ethanol and 1 g of concentrated hydrochloric acid and heated and stirred at 80° C. for 30 minutes. After the reaction, the reaction mixture was cooled to room temperature, and the precipitated crystal was filtered and successively washed with 5 g of ethanol and dried to obtain 1.1 g of a light yellow powder. This crystal was confirmed to be 4,5,6,7-tetrahydro-3-(2,6-dibromophenyl)-2(3H)-benzothiazole-thione by GC-MS. Yield 34%, M⁺=405

### Example 7

### Synthesis of 4,5,6,7-tetrahydro-3-(2,4,6-tribromophenyl)-2(3H)-benzothiazole-thione

A 100 mL flask purged with nitrogen was loaded with 10 g of 2,4,6-tribromoaniline and 20 g of dimethyl sulfoxide and the mixture was cooled to 5° C. while being stirred. The flask was further loaded with a solution obtained by dissolving 1.21 g of a sodium hydroxide powder in 1.1 g of water and the mixture was stirred for 10 minutes. Further, 2.3 g of carbon disulfide was added for 10 minutes and the mixture was heated to room temperature and stirred for 1 hour and cooled to 5° C. The resulting reaction solution was mixed with 4.0 g of 2-chlorocyclohexanone, heated to room temperature, and stirred for 1 hour. When the resulting reaction solution was mixed with 50 g of water and stirred for 30 minutes, the reaction solution was separated into a gum-like solid and a water layer and therefore, the water layer was removed by decantation and the gum-like solid was washed with 50 g of water. This solid was mixed with 30 g of ethanol and 8 g of concentrated hydrochloric acid and heated and stirred at 70° C. for 60 minutes. After the reaction, the reaction mixture was cooled to room temperature, and the precipitated crystal was filtered and successively washed with 5 g of ethanol and dried to obtain 7.4 g of a light yellow powder. This crystal was confirmed to be 4,5,6,7-tetrahydro-3-(2,4,6-tribromophenyl)-2(3H)-benzothiazole-thione by GC-MS. Yield 50%, M⁺=484

### Example 8

### Synthesis of 4-methyl-5-acetyl-3-(2,6-dibromophenyl)-2(3H)-thiazole-thione

A 100 mL flask purged with nitrogen was loaded with 5 g of 2,6-dibromoaniline and 10 g of dimethyl sulfoxide and the mixture was cooled to 5° C. while being stirred. The flask was further loaded with a solution obtained by dissolving 800 mg of a sodium hydroxide powder in 700 mg of water and the mixture was stirred for 10 minutes. Further, 1.51 g of carbon disulfide was added for 10 minutes and the mixture was heated to room temperature and stirred for 1 hour and cooled to 5° C. The resulting reaction solution was mixed with 2.7 g of 3-chloroacetylacetone, heated to room temperature, and stirred for 1 hour. When the resulting reaction solution was mixed with 10 g of water and stirred for 30 minutes, the reaction solution was separated into a gum-like solid and a water layer and therefore, the water layer was removed by decantation and the gum-like solid was washed with 10 g of water. This solid was mixed with 30 g of ethanol and 5 g of concentrated hydrochloric acid and heated and stirred at 80° C. for 30 minutes. After the reaction, the reaction mixture was cooled to room temperature, and the precipitated crystal was filtered and successively washed with 5 g of ethanol and dried to obtain 2.5 g of a light brown powder. This crystal was confirmed to be 4-methyl-5-acetyl-3-(2,6-dibromophenyl)-2(3H)-thiazole-thione by GC-MS. Yield 31%, M⁺=406

### Example 9

### Synthesis of 4,5,6,7-tetrahydro-3-[2,6-bis(3,5-di-tert-butylphenyl)phenyl]-2(3H)-benzothiazole-thione

A 100 mL flask purged with nitrogen was loaded with 300 mg of 4,5,6,7-tetrahydro-3-(2,6-dibromophenyl)-2(3H)-benzothiazole-thione synthesized in Example 6, 525 mg of 3,5-di-tert-butylphenylboronic acid, 10 g of tetrahydrofuran, 66 mg of (2-di-tert-butylphosphino)biphenyl, 680 mg of cesium fluoride, and 17 mg of palladium acetate and the mixture was heated to 50° C. and stirred for 18 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (after adsorption in 50 g of silica gel and elution with 300 mL of chloroform) and the solvent was removed by distillation to obtain 350 mg of a light yellow crystal. This crystal was confirmed to be 4,5,6,7-tetrahydro-3-[2,6-bis[3,5-di-tert-butylphenyl)phenyl]-2(3H)-benzothiazole-thione by ¹H-NMR and GC-MS. Yield 75%, M⁺=624

¹H-NMR (δ/ppm, CDCL3, on the basis of tetramethylsilane):1.3-1.7 (m, 44H), 7.2-7.6 (m, 9H)

### Example 10

### Synthesis of 4,5,6,7-tetrahydro-3-(2,6-diphenylphenyl)-2(3H)-benzothiazole-thione

A 100 mL flask purged with nitrogen was loaded with 300 mg of 4,5,6,7-tetrahydro-3-(2,6-dibromophenyl)-2(3H)-benzothiazole-thione synthesized in Example 6, 280 mg of phenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 18 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (after adsorption in 50 g of silica gel and elution with 300 mL of chloroform) and the solvent was removed by distillation to obtain 350 mg of a light yellow crystal. This crystal was confirmed to be a mixture containing 50% (gas chromatography area normalization method) of 4,5,6,7-tetrahydro-3-(2,6-diphenylphenyl)-2(3H)-benzothiazole-thione by GC-MS. Yield 59%, M⁺=399[0130]

### Example 11

### Synthesis of 4,5-dimethyl-3-[2,6-bis(3,5-di-trifluoromethylphenyl)phenyl]-2(3H)-thiazole-thione

A 100 mL flask purged with nitrogen was loaded with 500 mg of 4,5-dimethyl-3-(2,6-dibromophenyl)-2(3H)-benzothiazole-thione synthesized in Example 4, 1.02 g of 3,5-di-trifluoromethylphenylboronic acid, 10 g of tetrahydrofuran, 118 mg of (2-di-tert-butylphosphino)biphenyl, 1.2 g of cesium fluoride, and 30 mg of palladium acetate and the mixture was heated to 50° C. and stirred for 18 hours. During the stirring, at the moment after 9 hours, 1.02 g of 3,5-di-trifluoromethylphenylboronic acid was additionally added. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 850 mg of a light yellow crystal. This crystal was confirmed to be a mixture containing 30% (gas chromatography area normalization method) of 4,5-dimethyl-3-[2,6-bis(3,5-di-trifluoromethylphenyl)phenyl]-2(3H)-thiazole-thione by GC-MS. Yield 30%, M⁺=645

### Example 12

### Synthesis of 4,5-dimethyl-3-[2,6-bis(3,5-dimethoxyphenyl)phenyl]-2(3H)-thiazole-thione

A 100 mL flask purged with nitrogen was loaded with 500 mg of 4,5-dimethyl-3-(2,6-dibromophenyl)-2(3H)-thiazole-thione synthesized in Example 4, 240 mg of 3,5-dimethoxyphenylboronic acid, 10 g of tetrahydrofuran, 120 mg of (2-di-tert-butylphosphino)biphenyl, 1.2 g of cesium fluoride, and 30 mg of palladium acetate and the mixture was heated to 50° C. and stirred for 18 hours. During the stirring, at the moment after 9 hours, 240 mg of 3,5-dimethoxyphenylboronic acid was additionally added. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 580 mg of a light yellow oil. This crystal was confirmed to be a mixture containing 60% (gas chromatography area normalization method) of 4,5-dimethyl-3-[2,6-bis(3,5-dimethoxyphenyl)phenyl]-2(3H)-thiazole-thione by GC-MS.

Yield 53%, M⁺=493

### Example 13

### Synthesis of 3-[2,6-bis(3,5-di-tert-butylphenyl)phenyl]-4,5,6,7-tetrahydrobenzothiazolium chloride

A 100 mL flask purged with nitrogen was loaded with 350 mg of 4,5,6,7-tetrahydro-3-[2,6-bis(3,5-di-tert-butylphenyl)phenyl]-2(3H)-benzothiazole-thione synthesized in Example 9, 2 g of acetic acid, and 500 mg of 30 wt. % hydrogen peroxide water and the mixture was heated to 50° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 320 mg of a light brown powder. The powder was mixed with 10 g of toluene and dissolved matter was removed to obtain 230 mg of a white powder. This powder was confirmed to be 3-[2,6-bis(3,5-di-tert-butylphenyl)phenyl]-4,5,6,7-tetrahydrobenzothiazolium chloride by ¹H-NMR.

Yield 65%

¹H-NMR (δ/ppm, CDCL3, on the basis of tetramethylsilane): 1.23 (s, 36H), 1.4-1.7 (m, 8H), 6.8-7.7 (m, 9H), 10.91 (s, 1H)

### Example 14

### Synthesis of 3-(2,6-diphenylphenyl)-4,5,6,7-tetrahydrobenzothiazolium chloride

A 100 mL flask purged with nitrogen was loaded with 350 mg (purity 50%) of 4,5,6,7-tetrahydro-3-(2,6-phenylphenyl)-2(3H)-benzothiazole-thione synthesized in Example 10, 2 g of acetic acid, and 600 mg of 30 wt. % hydrogen peroxide water and the mixture was heated to 50° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 320 mg of a light brown powder. The powder was mixed with 10 g of toluene and dissolved matter was removed to obtain 150 mg of a light yellow powder. This powder was confirmed to be 3-(2,6-diphenylphenyl)-4,5,6,7-tetrahydrobenzothiazolium chloride by ¹H-NMR. Yield 85%

¹H-NMR (δ/ppm, CDCL3, on the basis of tetramethylsilane): 1.5-2.6 (m, 8H), 7.2-7.8 (m, 13H), 11.65 (s, 1H)

### Example 15

### Synthesis of 3-[2,6-bis(3,5-di-trifluoromethylphenyl)phenyl]-4,5-dimethylthiazolium chloride

A 100 mL flask purged with nitrogen was loaded with 850 mg (purity 30%) of 4,5-dimethyl-3-[2,6-bis(3,5-di-trifluoromethylphenyl)phenyl]-2(3H)-thiazole-thione synthesized in Example 11, 2 g of acetic acid, and 600 mg of 30 wt. % hydrogen peroxide water and the mixture was heated to 50° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 850 mg of a light brown oil. The oil was mixed with 10 g of toluene and dissolved matter was removed to obtain 250 mg of a light brown powder. The obtained powder was confirmed to be 3-[2,6-bis(3,5-di-trifluoromethylphenyl)phenyl]-4,5-dimethylthiazolium chloride by ¹H-NMR. Yield 98%

¹H-NMR (δ/ppm, CDCL3, on the basis of tetramethylsilane): 1.95 (s, 3H), 2.35 (s, 3H), 7.2-8.1 (m, 9H), 10.96 (s, 1H)

### Example 16

### Synthesis of 3-[2,6-bis(3,5-dimethoxyphenyl)phenyl]-4,5-dimethylthiazolium chloride

A 100 mL flask purged with nitrogen was loaded with 580 mg (purity 60%) of 4,5-dimethyl-3-[2,6-bis(3,5-dimethoxyphenyl)phenyl]-2(3H)-thiazole-thione synthesized in Example 12, 2 g of acetic acid, and 600 mg of 30 wt. % hydrogen peroxide water and the mixture was heated to 50° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 520 mg of a light brown oil. The oil was mixed with 10 g of toluene and dissolved matter was removed to obtain 300 mg of a light brown powder. The obtained powder was confirmed to be 3-[2,6-bis(3,5-dimethoxyphenyl)phenyl]-4,5-dimethylthiazolium chloride by ¹H-NMR. Yield 86%

¹H-NMR (δ/ppm, CDCL3, on the basis of tetramethylsilane): 2.02 (s, 3H), 2.45 (s, 3H), 3.78 (s, 12H), 7.4-7.9 (m, 9H), 11.02 (s, 1H)

### Example 17

### Synthesis of 3-(2,6-dibromophenyl)-4,5-dimethylthiazolium chloride

A 100 mL flask purged with nitrogen was loaded with 300 mg of 4,5-dimethyl-3-(2,6-dibromophenyl]-2(3H)-thiazole-thione synthesized in Example 4, 2 g of acetic acid, and 500 mg of 30 wt. % hydrogen peroxide water and the mixture was heated to 50° C. and stirred for 60 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 300 mg of a light brown solid material. The obtained solid material was confirmed to be 3-(2,6-dibromophenyl)-4,5-dimethylthiazolium chloride by ¹H-NMR. Yield 98%

¹H-NMR (δ/ppm, CDCL3, on the basis of tetramethylsilane): 2.21 (s, 3H), 2.76 (s, 3H), 7.4-7.9 (m, 3H), 11.28 (s, 1H)

### Example 18

### Synthesis of 3-(2,6-dibromophenyl)-4,5,6,7-tetrahydrobenzothiazolium chloride

A 100 mL flask purged with nitrogen was loaded with 300 mg of 4,5,6,7-tetrahydro-3-(2,6-dibromophenyl)-2(3H)-benzothiazole-thione synthesized in Example 6, 2 g of acetic acid, and 600 mg of 30 wt. % hydrogen peroxide water and the mixture was heated to 60° C. and stirred for 60 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 390 mg of a light brown tar-like material. This tar-like material was solidified after being left at room temperature. The obtained material was mixed with 3 g of acetone and the produced crystal was filtered and dried to obtain 150 mg of a white powder. This powder was confirmed to be 3-(2,6-dibromophenyl)-4,5,6,7-tetrahydrobenzothiazolium chloride by ¹H-NMR. Yield 38%

¹H-NMR (δ/ppm, CDCL3, on the basis of tetramethylsilane): 1.75-2.02 (m, 4H), 2.49 (bs, 2H), 3.05 (bs, 2H), 7.49 (dd, 1H), 7.82 (d, 2H), 11.49 (s, 1H)

### Example 19

### Synthesis of 3-(2,4,6-tribromophenyl)-4,5,6,7-tetrahydrobenzothiazolium chloride

A 100 mL flask purged with nitrogen was loaded with 500 mg of 4,5,6,7-tetrahydro-3-(2,4,6-tribromophenyl)-2(3H)-benzothiazole-thione synthesized in Example 7, 4 g of acetic acid, and 1.0 g of 30 wt. % hydrogen peroxide water and the mixture was heated to 60° C. and stirred for 2 hours. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 390 mg of a light brown tar-like material. The obtained tar-like material was mixed with 3 g of acetone and the produced crystal was filtered and dried to obtain 50 mg of a white powder. This powder was confirmed to be 3-(2,4,6-tribromophenyl)-4,5,6,7-tetrahydrobenzothiazolium chloride by ¹H-NMR. Yield 10%

¹H-NMR (δ/ppm, CDCL3, on the basis of tetramethylsilane): 1.75-2.02 (m, 4H), 2.47 (bs, 2H), 3.02 (bs, 2H), 7.99 (s, 2H), 11.66 (s, 1H)

### Example 20

A 50 mL Schlenk flask purged with nitrogen was loaded with 3.4 g of 3-methylthiopropanal, 990 mg of paraformaldehyde, 20 mg (0.5 mol %) of 3-[2,6-bis(3,5-di-tert-butylphenyl)phenyl]-4,5-dimethylthiazolium chloride obtained in Example 2, 7 g of toluene, and 1 g of dry ice and thereafter, generated gaseous carbon dioxide was discharged to lower the pressure to normal pressure. After the obtained mixture was heated to 50° C. and bubbling of carbon dioxide gas subsided, a solution obtained by dissolving 5 mg of 1,8-diazabicyclo[5.4.0]-7-undecene in 100 mg of toluene was added while being stirred and the obtained mixture was stirred at 50° C. for 2 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 55% and 33% of 3-methylthiopropanal, a raw material, was recovered. The yield of 1,6-dimethylthio-4-hydroxy-3-hexanone, a homo-coupling product, was 12%. GC-MS was employed for identifying 1,6-dimethylthio-4-hydroxy-3-hexanone.

MS (m/z): 208 (M⁺)

### Comparative Example 1

A 50 mL Schlenk flask purged with nitrogen was loaded with 1.2 g of 3-methylthiopropanal, 530 mg of paraformaldehyde, 30 mg (1 mol %) of 3-benzylthiazolium bromide, 2.5 g of toluene, and 500 mg of dry ice and thereafter, generated gaseous carbon dioxide was discharged to lower the pressure to normal pressure. After the obtained mixture was heated to 50° C. and bubbling of carbon dioxide gas subsided, a solution obtained by dissolving 18 mg of 1,8-diazabicyclo[5.4.0]-7-undecene in 100 mg of toluene was added while being stirred and the obtained mixture was stirred at 50° C. for 2 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 4% and 60% of 3-methylthiopropanal, a raw material, was recovered. The yield of 1,6-dimethylthio-4-hydroxy-3-hexanone, a homo-coupling product, was 1%.

### Example 21

A 50 mL Schlenk flask purged with nitrogen was loaded with 1.7 g of 3-methylthiopropanal, 2.1 g of 35 wt. % formalin water, 20 mg of 3-[2,6-bis(3,5-di-tert-butylphenyl)phenyl]-4,5-dimethylthiazolium chloride obtained in Example 2, and 4 g of toluene. After 1 g of dry ice was added to the flask, generated gaseous carbon dioxide was discharged to lower the pressure to normal pressure. After the obtained mixture was heated to 50° C. and bubbling of carbon dioxide gas subsided, a mixed solution containing 5 mg of 1,8-diazabicyclo[5.4.0]-7-undecene and 100 mg of toluene was added while being stirred and the obtained mixture was stirred at 50° C. for 14 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 75% and 21% of 3-methylthiopropanal, a raw material, was recovered. The yield of 1,6-dimethylthio-4-hydroxy-3-hexanone, a homo-coupling product, was 4%.

The production ratio after 6 hour-reaction time was as follows.

4-(methyl)-2-oxo-1-butanol: 61%
Raw material 3-methylthiopropanal: 36%
1,6-dimethylthio-4-hydroxy-3-hexanone: 2%

### Example 22

A 50 mL Schlenk flask purged with nitrogen was loaded with 2.07 g of 3-methylthiopropanal, 900 mg of paraformaldehyde, 19 mg of 3-[(2,6-diphenyl-3,5-diisopropyl)phenyl]-4,5-dimethylthiazolium chloride obtained in Example 3, 5 g of toluene, and 1 g of dry ice and thereafter, generated gaseous carbon dioxide was discharged to lower the pressure to normal pressure. After the obtained mixture was heated to 50° C. and bubbling of carbon dioxide gas subsided, a solution obtained by dissolving 8 mg of 1,8-diazabicyclo[5.4.0]-7-undecene in 500 mg of toluene was added while being stirred and the obtained mixture was stirred at 50° C. for 4 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 27% and 59% of 3-methylthiopropanal, a raw material, was recovered. The yield of 1,6-dimethylthio-4-hydroxy-3-hexanone, a homo-coupling product, was 10%.

### Example 23

A 50 mL Schlenk flask purged with nitrogen was loaded with 2.75 g of 3-methylthiopropanal, 1.2 g of paraformaldehyde, 10 mg of 3-[2,6-di(phenyl)phenyl]-4,5-dimethylthiazolium chloride obtained in Example 1, 6 g of toluene, and 1 g of dry ice and thereafter, generated gaseous carbon dioxide was discharged to lower the pressure to normal pressure. After the obtained mixture was heated to 50° C. and bubbling of carbon dioxide gas subsided, a solution obtained by dissolving 4 mg of 1,8-diazabicyclo[5.4.0]-7-undecene in 100 mg of toluene was added while being stirred and the obtained mixture was stirred at 50° C. for 4 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 28% and 65% of 3-methylthiopropanal, a raw material, was recovered. The yield of 1,6-dimethylthio-4-hydroxy-3-hexanone, a homo-coupling product, was 7%.

### Example 24

A 50 mL Schlenk flask purged with nitrogen was loaded with 1.38 g of 3-methylthiopropanal, 400 mg of paraformaldehyde, 40 mg of 3-[2,6-di(phenyl)phenyl]-4,5-dimethylthiazolium chloride obtained in Example 1, and 2.8 g of tetrahydrofuran. The obtained mixture was heated to 40° C., mixed with 16 mg of 1,8-diazabicyclo[5.4.0]-7-undecene, and then stirred at 40° C. for 1.5 hours. At that moment, since it was confirmed that 3-methylthiopropanal, a raw material, disappeared by gas chromatographic analysis, 1.38 g of 3-methylthiopropanal and 400 mg of paraformaldehyde were additionally added and the mixture was further stirred at 40° C. for 1 hour. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 47% and 20% of 3-methylthiopropanal, a raw material, was recovered. The yield of 1,6-dimethylthio-4-hydroxy-3-hexanone, a homo-coupling product, was 33%.

### Example 25

A 50 mL Schlenk flask purged with nitrogen was loaded with 3.07 g of propanal, 10 mg of 3-[2,6-di(phenyl)phenyl]-4,5-dimethylthiazolium chloride obtained in Example 1, 6 g of toluene, and 1 g of dry ice and thereafter, generated gaseous carbon dioxide was discharged to lower the pressure to normal pressure. After the obtained mixture was heated to 40° C. and bubbling of carbon dioxide gas subsided, a mixed solution containing 4 mg of 1,8-diazabicyclo[5.4.0]-7-undecene and 100 mg of toluene was added while being stirred and the obtained mixture was stirred at 40° C. for 4 hours. The obtained reaction mixture was cooled to room temperature to obtain a reaction mixture containing 4-hydroxy-3-hexanone. According to analysis by a gas chromatography internal standard method, the yield of 4-hydroxy-3-hexanone was 22% and 75% of propanal was recovered.

### Example 26

A 50 mL Schlenk flask purged with nitrogen was loaded with 841 mg of benzaldehyde, 480 mg of paraformaldehyde, 30 mg of 3-[2,6-di(phenyl)phenyl]-4,5-dimethylthiazolium chloride obtained in Example 1, and 3 g of toluene. The obtained mixture was heated to 60° C., mixed with 24 mg of 1,8-diazabicyclo[5.4.0]-7-undecene while being stirred, and then stirred at 60° C. for 6 hours. The obtained reaction mixture was cooled to room temperature to obtain a reaction mixture containing 2-hydroxy-1-phenyl-ethanone. According to analysis by a gas chromatography internal standard method, the yield of 2-hydroxy-1-phenyl-ethanone was 21% and 77% of benzaldehyde was recovered.

### Example 27

A 50 mL Schlenk flask purged with nitrogen was loaded with 1.24 g of 3-methylthiopropanal, 540 mg of paraformaldehyde, 30 mg of 3-[2,6-bis(3,5-di-tert-butylphenyl)phenyl]-4,5,6,7-tetrahydrobenzothiazolium chloride obtained in Example 13, and 3 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 8 mg of 1,8-diazabicyclo[5.4.0]-7-undecene and 100 mg of toluene was added while being stirred and the obtained mixture was stirred at 50° C. for 2 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 51% and 11% of 3-methylthiopropanal, a raw material, was recovered. The yield of 1,6-dimethylthio-4-hydroxy-3-hexanone, a homo-coupling product, was 36%.

### Example 28

A 50 mL Schlenk flask purged with nitrogen was loaded with 5.0 g of 3-methylthiopropanal, 3.0 g of 35 wt. % formalin water, 30 mg of 3-[2,6-bis(3,5-di-tert-butylphenyl)phenyl]-4,5,6,7-tetrahydrobenzothiazolium chloride obtained in Example 13, and 10 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 8 mg of 1,8-diazabicyclo[5.4.0]-7-undecene and 100 mg of toluene was added while being stirred and the obtained mixture was stirred at 50° C. for 16 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 57% and 33% of 3-methylthiopropanal, a raw material, was recovered. The yield of 1,6-dimethylthio-4-hydroxy-3-hexanone, a homo-coupling product, was 7%.

The production ratio after 6 hour-reaction time was as follows.

4-(methylthio)-2-oxo-1-butanol: 45%
Raw material 3-methylthiopropanal: 54%
1,6-dimethylthio-4-hydroxy-3-hexanone: 0%

### Example 29

A 100 mL four-mouth flask purged with nitrogen was loaded with 10 g of 3-methylthiopropanal, 15 g of 35 wt. % formalin water, 30 mg of 3-[2,6-bis(3,5-di-tert-butylphenyl)phenyl]-4,5,6,7-tetrahydrobenzothiazolium chloride obtained in Example 13, and 20 g of toluene. After 1 g of dry ice was added to the flask, generated gaseous carbon dioxide was discharged to lower the pressure to normal pressure. After the obtained mixture was heated to 50° C. and bubbling of carbon dioxide gas subsided, a mixed solution containing 8 mg of 1,8-diazabicyclo[5.4.0]-7-undecene and 100 mg of toluene was added while being stirred and the obtained mixture was stirred at 50° C. for 16 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 35% and 64% of 3-methylthiopropanal, a raw material, was recovered. The yield of 1,6-dimethylthio-4-hydroxy-3-hexanone, a homo-coupling product, was 0.5%.

The production ratio after 8 hour-reaction time was as follows.

4-(methylthio)-2-oxo-1-butanol: 25%
Raw material 3-methylthiopropanal: 74%
1,6-dimethylthio-4-hydroxy-3-hexanone: 0%

### Example 30

A 50 mL Schlenk flask purged with nitrogen was loaded with 960 mg of 3-methylthiopropanal, 1.1 g of 35 wt. % formalin water, 30 mg of 3-[2,6-bis(3,5-di-trifluoromethylphenyl)phenyl]-4,5-dimethylthiazolium chloride obtained in Example 15, and 2 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 7 mg of 1,8-diazabicyclo[5.4.0]-7-undecene and 100 mg of toluene was added while being stirred and the obtained mixture was stirred at 50° C. for 6 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 53% and 26% of 3-methylthiopropanal, a raw material, was recovered. The yield of 1,6-dimethylthio-4-hydroxy-3-hexanone, a homo-coupling product, was 4%.

### Example 31

A 50 mL Schlenk flask purged with nitrogen was loaded with 1.25 g of 3-methylthiopropanal, 1.5 g of 35 wt. % formalin water, 30 mg of 3-[2,6-bis(3,5-dimethoxyphenyl)phenyl]-4,5-dimethylthiazolium chloride obtained in Example 16, and 2 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 9 mg of 1,8-diazabicyclo[5.4.0]-7-undecene and 100 mg of toluene was added while being stirred and the obtained mixture was stirred at 50° C. for 6 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 10% and 79% of 3-methylthiopropanal, a raw material, was recovered. The yield of 1,6-dimethylthio-4-hydroxy-3-hexanone, a homo-coupling product, was 3%.

### Example 32

A 50 mL Schlenk flask purged with nitrogen was loaded with 1.54 g of 3-methylthiopropanal, 1.2 g of 35 wt. % formalin water, 30 mg of 3-(2,6-diphenylphenyl)-4,5,6,7-tetrahydrobenzothiazolium chloride obtained in Example 14, and 3 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 11 mg of 1,8-diazabicyclo[5.4.0]-7-undecene and 100 mg of toluene was added while being stirred and the obtained mixture was stirred at 50° C. for 8 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 58% and 36% of 3-methylthiopropanal, a raw material, was recovered. The yield of 1,6-dimethylthio-4-hydroxy-3-hexanone, a homo-coupling product, was 2%.

### Example 33

A 50 mL Schlenk flask purged with nitrogen was loaded with 1.9 g of 3-methylthiopropanal, 2.2 g of 35 wt. % formalin water, 15 mg of 3-(2,6-dibromophenyl)-4,5,6,7-tetrahydrobenzothiazolium chloride obtained in Example 18, and 4 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 6 mg of 1,8-diazabicyclo[5.4.0]-7-undecene and 100 mg of toluene was added while being stirred and the obtained mixture was stirred at 50° C. for 12 hours. The obtained reaction mixture was cooled to room temperature to obtain a reaction mixture containing 4-(methylthio)-2-oxo-1-butanol.

According to analysis by a gas chromatography internal standard method, the yield of 4-(methylthio)-2-oxo-1-butanol, a cross-coupling product, was 19% and 64% of 3-methylthiopropanal, a raw material, was recovered. The yield of 1,6-dimethylthio-4-hydroxy-3-hexanone, a homo-coupling product, was 0%.

### Example 34

A 50 mL Schlenk flask purged with nitrogen was loaded with 3.2 g of 3-methylthiopropanal, 3.6 g of 35 wt. % formalin water, 30 mg of 3-(2,4,6-tribromophenyl)-4,5,6,7-tetrahydrobenzothiazolium chloride obtained in Example 19, and 7 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 10 mg of 1,8-diazabicyclo[5.4.0]-7-undecene and 100 mg of toluene was added while being stirred and the obtained mixture was stirred at 50° C. for 6 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 6% and 88% of 3-methylthiopropanal, a raw material, was recovered. The yield of 1,6-dimethylthio-4-hydroxy-3-hexanone, a homo-coupling product, was 0%.

### Example 35

A 50 mL Schlenk flask purged with nitrogen was loaded with 921 mg of benzaldehyde, 480 mg of paraformaldehyde, 23 mg of 3-(2,6-dibromophenyl)-4,5,6,7-tetrahydrobenzothiazolium chloride obtained in Example 18, and 2 g of toluene. The obtained mixture was heated to 50° C., mixed with 12 mg of 1,8-diazabicyclo[5.4.0]-7-undecene, and then stirred at 50° C. for 6 hours. The obtained reaction mixture was cooled to room temperature to obtain a reaction mixture containing 2-hydroxy-1-phenyl-ethanone. According to analysis by a gas chromatography internal standard method, the yield of 2-hydroxy-1-phenyl-ethanone, a cross-coupling product, was 26%, the yield of 2-hydroxy-1-phenyl-2-phenylethanone, a homo-coupling product was 16%, and 58% of benzaldehyde, a raw material, was recovered.

### Example 36

### Synthesis of 4,5,6,7-tetrahydro-3-(2-bromo-6-methylophenyl)-2(3H)-benzothiazole-thione

A 100 mL flask purged with nitrogen was loaded with 5 g of 2-bromo-6-methylaniline and 10 g of dimethyl sulfoxide and the mixture was cooled to 5° C. while being stirred. The flask was further loaded with a solution containing 1.07 g of a sodium hydroxide powder dissolved in 1.0 g of water and the mixture was stirred for 10 minutes. Further, 2.0 g of carbon disulfide was added for 10 minutes and the mixture was heated to room temperature and stirred for 1 hour and cooled to 5° C. The resulting reaction solution was mixed with 3.56 g of 2-chlorocyclohexanone, heated to room temperature, and stirred for 1 hour. When the resulting reaction solution was mixed with 50 g of water and stirred for 30 minutes, the reaction solution was separated into a gum-like solid and a water layer and therefore, the water layer was removed by decantation and the gum-like solid was washed with 50 g of water. This solid was mixed with 20 g of ethanol and 2 g of concentrated hydrochloric acid and heated and stirred at 80° C. for 30 minutes. After the reaction, the reaction mixture was cooled to room temperature, and the precipitated crystal was filtered and successively washed with 10 g of ethanol and dried to obtain 4.4 g of a light yellow powder. This crystal was confirmed to be 4,5,6,7-tetrahydro-3-(2-bromo-6-methylphenyl)-2(3H)-benzothiazole-thione by GC-MS. Yield 48%, M⁺=340

### Example 37

### Synthesis of 4,5,6,7-tetrahydro-3-[2-(3,5-di-tert-butylphenyl)-6-methylphenyl]-2(3H)-benzothiazole-thione

A 100 mL flask purged with nitrogen was loaded with 300 mg of 4,5,6,7-tetrahydro-3-(2-bromo-6-methylphenyl)-2(3H)-benzothiazole-thione synthesized in Example 36, 413 mg of 3,5-di-tert-butylphenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 18 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 380 mg of a light yellow crystal.

This crystal was confirmed to be 4,5,6,7-tetrahydro-3-[2-(3,5-di-tert-butylphenyl)-6-methylphenyl)-2(3H)-benzothiazole-thione by GC-MS. Yield 95%, M⁺=449

### Example 38

### Synthesis of 3-[2-(3,5-di-tert-butylphenyl)-6-methylphenyl]-4,5,6,7-tetrahydrobenzothiazolium chloride

A 100 mL flask purged with nitrogen was loaded with 380 mg of 4,5,6,7-tetrahydro-3-[2-(3,5-di-tert-butylphenyl)-6-methylphenyl]-2(3H)-benzothiazole-thione synthesized in Example 37, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 340 mg of a light brown powder. This powder was confirmed to be 3-[2-(3,5-di-tert-butylphenyl)-6-methylphenyl]-4,5,6,7-tetrahydrobenzothiazolium chloride by ¹H-NMR. Yield 89%

¹H-NMR (δ/ppm, CDCL3, on the basis of tetramethylsilane): 1.22 (s, 18H), 1.4-1.7 (m, 8H), 2.22 (s, 3H), 6.8-7.6 (m, 6H), 11.10 (s, 1H)

### Example 39

A 50 mL Schlenk flask purged with nitrogen was loaded with 205 mg of benzaldehyde, 460 mg of 38 wt. % of formalin water, 30 mg of 3-[2-(3,5-di-tert-butylphenyl)-6-methylphenyl]-4,5,6,7-tetrahydrobenzothiazolium chloride obtained in Example 38, 2 g of toluene, and 500 mg of dry ice and thereafter, generated gaseous carbon dioxide was discharged to lower the pressure to normal pressure. After the obtained mixture was heated to 40° C. and bubbling of carbon dioxide gas subsided, a solution obtained by dissolving 20 mg of 1,8-diazabicyclo[5.4.0]-7-undecene in 100 mg of toluene was added while being stirred and the obtained mixture was stirred at 40° C. for 6 hours. The obtained reaction mixture was cooled to room temperature to obtain a reaction mixture containing 2-hydroxy-1-phenyl-ethanone. According to analysis by a gas chromatography internal standard method, the yield of 2-hydroxy-1-phenyl-ethanone was 84% and 15% of benzaldehyde was recovered.

### Example 40

A 50 mL Schlenk flask purged with nitrogen was loaded with 6.9 g of 3-methylthiopropanal, 7.8 g of 35 wt. % formalin water, 30 mg of 3-[2-(3,5-di-tert-butylphenyl)-6-methylphenyl]-4,5,6,7-tetrahydrobenzothiazolium chloride obtained in Example 38, and 14 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 10 mg of 1,8-diazabicyclo[5.4.0]-7-undecene and 100 mg of toluene was added while being stirred and the obtained mixture was stirred at 50° C. for 8 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 8% and 91% of 3-methylthiopropanal, a raw material, was recovered. The yield of 1,6-dimethylthio-4-hydroxy-3-hexanone, a homo-coupling product, was 0.5%.

### Example 41

### Synthesis of 4,5,6,7-tetrahydro-3-[2,6-bis(3-tert-butylphenyl)phenyl]-2(3H)-benzothiazole-thione

A 100 mL flask purged with nitrogen was loaded with 300 mg of 4,5,6,7-tetrahydro-3-(2,6-dibromophenyl)-2(3H)-benzothiazole-thione synthesized the same as in Example 6, 397 mg of 3-tert-butylphenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 360 mg of a light yellow crystal. This crystal was confirmed to be 4,5,6,7-tetrahydro-3-[2,6-bis[3-tert-butylphenyl)phenyl]-2(3H)-benzothiazole-thione by GC-MS. Yield 95%, M⁺=511

### Example 42

### Synthesis of 3-[2,6-bis(3-tert-butylphenyl)phenyl]-4,5,6,7-tetrahydrobenzothiazolium chloride

A 100 mL flask purged with nitrogen was loaded with 360 mg of 4,5,6,7-tetrahydro-3-[2,6-bis(3-tert-butylphenyl)phenyl]-2(3H)-benzothiazole-thione synthesized in Example 41, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 360 mg of a light brown powder. This powder was confirmed to be 3-[2,6-bis(3-tert-butylphenyl)phenyl]-4,5,6,7-tetrahydrobenzothiazolium chloride by ¹H-NMR.

Yield 98%

¹H-NMR (δ/ppm, CDCL3, on the basis of tetramethylsilane): 1.26 (s, 18H), 1.4-2.5 (m, 8H), 7.0-7.6 (m, 11H), 11.20 (s, 1H)

### Example 43

### Synthesis of 4,5,6,7-tetrahydro-3-[2,6-bis(3,5-dichlorophenyl)phenyl)-2(3H)-benzothiazole-thione

A 100 mL flask purged with nitrogen was loaded with 300 mg of 4,5,6,7-tetrahydro-3-(2,6-dibromophenyl)-2(3H)-benzothiazole-thione synthesized the same as in Example 6, 430 mg of 3,5-dichlorophenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 380 mg of a light yellow crystal. This crystal was confirmed to be 4,5,6,7-tetrahydro-3-[2,6-bis(3,5-dichlorophenyl)phenyl]-2(3H)-benzothiazole-thione by GC-MS. Yield 96%, M⁺=537

### Example 44

### Synthesis of 3-[2,6-bis(3,5-dichlorophenyl)phenyl]-4,5,6,7-tetrahydrobenzothiazolium chloride

A 100 mL flask purged with nitrogen was loaded with 380 mg of 4,5,6,7-tetrahydro-3-[2,6-bis(3,5-dichlorophenyl)phenyl]-2(3H)-benzothiazole-thione synthesized in Example 43, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue. At that time, a formed insoluble white crystal was removed by filtration and the filtrate was removed by distillation. The obtained residue was mixed with 10 g of chloroform and 10 g of saturated salt water and extracted and separated by a separatory funnel. The solvent was removed by distillation from the obtained chloroform layer to obtain 300 mg of a light brown powder. This powder was confirmed to be 3-[2,6-bis(3,5-dichlorophenyl)phenyl]-4,5,6,7-tetrahydrobenzothiazolium chloride by ¹H-NMR. Yield 79%

¹H-NMR (δ/ppm, CDCL3, on the basis of tetramethylsilane): 1.6-2.7 (m, 8H), 7.0-7.8 (m, 9H), 12.40 (s, 1H)

### Example 45

### Synthesis of 4,5,6,7-tetrahydro-3-[2,6-bis(3.5-dibromophenyl)phenyl)-2(3H)-benzothiazole-thione

A 100 mL flask purged with nitrogen was loaded with 300 mg of 4,5,6,7-tetrahydro-3-(2,6-dibromophenyl)-2(3H)-benzothiazole-thione synthesized the same as in Example 6, 630 mg of 3,5-dibromophenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 8 hours. At that time, 400 mg of 3,5-dibromophenylboronic acid was additionally added and the mixture was stirred further for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 420 mg of a light yellow crystal. This crystal was confirmed to be 4,5,6,7-tetrahydro-3-[2,6-bis(3,5-dibromophenyl)phenyl]-2(3H)-benzothiazole-thione by GC-MS. Yield 79%, M⁺=715

### Example 46

### Synthesis of 3-[2,6-bis(3,5-dibromophenyl)phenyl]-4,5,6,7-tetrahydrobenzothiazolium chloride

A 100 mL flask purged with nitrogen was loaded with 420 mg of 4,5,6,7-tetrahydro-3-[2,6-bis(3,-dibromophenyl)phenyl]-2(3H)-benzothiazole-thione synthesized in Example 45, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue. At that time, a formed insoluble white crystal was removed by filtration and the filtrate was removed by distillation. The obtained residue was mixed with 10 g of chloroform and 10 g of saturated salt water and extracted and separated by a separatory funnel. The solvent was removed by distillation from the obtained chloroform layer to obtain 310 mg of a light brown powder. This powder was confirmed to be 3-[2,6-bis(3,5-dibromophenyl)phenyl]-4,5,6,7-tetrahydrobenzothiazolium chloride by ¹H-NMR. Yield 73%

¹H-NMR (δ/ppm, CDCL3, on the basis of tetramethylsilane):

1.6-2.7 (m, 8H), 7.2-7.8 (m, 9H), 12.17 (s, 1H)

### Example 47

### Synthesis of 4,5,6,7-tetrahydro-3-(2,6-dibromo-4-methylphenyl)-2(3H)-benzothiazole-thione

A 200 mL flask purged with nitrogen was loaded with 5 g of 2,6-dibromo-4-methylaniline and 10 g of dimethyl sulfoxide and the mixture was cooled to 5° C. while being stirred. The flask was further loaded with 755 mg of a sodium hydroxide powder dissolved in 800 mg of water and the mixture was stirred for 10 minutes. Further, 1.4 g of carbon disulfide was added for 10 minutes and the mixture was heated to room temperature and stirred for 1 hour and cooled to 5° C. The resulting reaction solution was mixed with 2.5 g of 2-chlorocyclohexanone, heated to room temperature, and stirred for 1 hour. When the resulting reaction solution was mixed with 50 g of water and stirred for 30 minutes, the reaction solution was separated into a gum-like solid and a water layer and therefore, the water layer was removed by decantation and the gum-like solid was washed with 50 g of water. This solid was mixed with 20 g of ethanol and 2 g of concentrated hydrochloric acid and heated and stirred at 60° C. for 30 minutes. After the reaction, the reaction mixture was cooled to room temperature, and the precipitated crystal was filtered and successively washed with 10 g of ethanol and dried to obtain 2.5 g of a light yellow powder. This crystal was confirmed to be 4,5,6,7-tetrahydro-3-(2,6-dibromo-4-methylphenyl)-2(3H)-benzothiazole-thione by GC-MS. Yield 32%, M⁺=419

### Example 48

### Synthesis of 4,5,6,7-tetrahydro-3-[2,6-bis(3,5-di-tert-butylphenyl)-4-methylphenyl]-2(3H)-benzothiazole-thione

A 100 mL flask purged with nitrogen was loaded with 300 mg of 4,5,6,7-tetrahydro-3-(2,6-dibromo-4-methylphenyl)-2(3H)-benzothiazole-thione synthesized in Example 47, 502 mg of 3,5-di-tert-butylphenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 8 hours. At that time, 400 mg of 3,5-di-tert-butylphenylboronic acid was additionally added and the mixture was stirred at 80° C. further for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 350 mg of a light yellow crystal. This crystal was confirmed to be 4,5,6,7-tetrahydro-3-[2,6-bis(3,5-di-tert-butylphenyl)-4-methylphenyl)-2(3H)-benzothiazole-thione by GC-MS. Yield 77%, M⁺=637

### Example 49

### Synthesis of 3-[2,6-bis(3,5-di-tert-butylphenyl)-4-methylphenyl]-4,5,6,7-tetrahydrobenzothiazolium chloride

A 100 mL flask purged with nitrogen was loaded with 350 mg of 4,5,6,7-tetrahydro-3-[2,6-bis(3,5-di-tert-butylphenyl)-4-methylphenyl]-2(3H)-benzothiazole-thione synthesized in Example 48, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 330 mg of a light brown powder. This powder was confirmed to be 3-[2,6-bis(3,5-di-tert-butylphenyl)-4-methylphenyl]-4,5,6,7-tetrahydrobenzothiazolium chloride by ¹H-NMR. Yield 94%

¹H-NMR (δ/ppm, CDCL3, on the basis of tetramethylsilane): 1.26 (s, 36H), 1.6-2.6 (m, 8H), 2.52 (s, 3H), 7.0-7.7 (m, 8H), 10.85 (s, 1H)

### Example 50

### Synthesis of 4,5,6,7-tetrahydro-3-(2,6-dibromo-4-chlorophenyl)-2(3H)-benzothiazole-thione

A 200 mL flask purged with nitrogen was loaded with 10 g of 2,6-dibromo-4-chloroaniline and 20 g of dimethyl sulfoxide and the mixture was cooled to 5° C. while being stirred. The flask was further loaded with 1.4 g of a sodium hydroxide powder dissolved in 1.2 g of water and the mixture was stirred for 10 minutes. Further, 2.7 g of carbon disulfide was added for 10 minutes and the mixture was heated to room temperature and stirred for 1 hour and cooled to 5° C. The resulting reaction solution was mixed with 4.6 g of 2-chlorocyclohexanone, heated to room temperature, and stirred for 1 hour. When the resulting reaction solution was mixed with 50 g of water and stirred for 30 minutes, the reaction solution was separated into a gum-like solid and a water layer and therefore, the water layer was removed by decantation and the gum-like solid was washed with 50 g of water. This solid was mixed with 50 g of ethanol and 5 g of concentrated hydrochloric acid and heated and stirred at 60° C. for 30 minutes. After the reaction, the reaction mixture was cooled to room temperature, and the precipitated crystal was filtered and successively washed with 10 g of ethanol and dried to obtain 8.9 g of a light yellow powder. This crystal was confirmed to be 4,5,6,7-tetrahydro-3-(2,6-dibromo-4-chlorophenyl)-2(3H)-benzothiazole-thione by GC-MS. Yield 58%, M⁺=439

### Example 51

### Synthesis of 4,5,6,7-tetrahydro-3-[2,6-bis(3,5-di-tert-butylphenyl)-4-chlorophenyl]-2(3H)-benzothiazole-thione

A 100 mL flask purged with nitrogen was loaded with 300 mg of 4,5,6,7-tetrahydro-3-(2,6-dibromo-4-chlorophenyl)-2(3H)-benzothiazole-thione synthesized in Example 50, 480 mg of 3,5-di-tert-butylphenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 420 mg of a light yellow crystal. This crystal was confirmed to be 4,5,6,7-tetrahydro-3-[2,6-bis(3,5-di-tert-butylphenyl)-4-chlorophenyl)-2(3H)-benzothiazole-thione by GC-MS. Yield 94% M⁺=658

### Example 52

### Synthesis of 3-[2,6-bis(3,5-di-tert-butylphenyl)-4-chlorophenyl]-4,5,6,7-tetrahydrobenzothiazolium chloride

A 100 mL flask purged with nitrogen was loaded with 420 mg of 4,5,6,7-tetrahydro-3-[2,6-bis(3,5-di-tert-butylphenyl)-4-chlorophenyl]-2(3H)-benzothiazole-thione synthesized in Example 51, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 410 mg of a light brown powder.

This powder was confirmed to be 3-[2,6-bis(3,5-di-tert-butylphenyl)-4-chlorophenyl]-4,5,6,7-tetrahydrobenzothiazolium chloride by ¹H-NMR. Yield 97%

¹H-NMR (δ/ppm, CDCL3, on the basis of tetramethylsilane): 1.26 (s, 36H), 1.6-2.6 (m, 8H), 7.0-7.7 (m, 8H), 11.30 (s, 1H)

### Example 53

### Synthesis of 4,5-dimethyl-3-(2,6-diphenylphenyl)-2(3H)-thiazole-thione

A 50 mL flask purged with nitrogen was loaded with 5 mg of palladium acetate, 20 mg of (2-di-tert-butylphosphino)biphenyl, and 5 g of tetrahydrofuran and the mixture was observed at 20° C. for 10 minutes. Further, the flask was loaded with 140 mg of zinc chloride and 1 mL of a tetrahydrofuran solution of phenylmagnesium bromide (2 mol/L concentration, produced by Tokyo Kasei Kogyo Co., Ltd.) and the mixture was stirred at room temperature for 10 minutes. This mixed solution was mixed with 200 mg of 4,5-dimethyl-3-(2,6-dibromophenyl)-2(3H)-thiazole-thione synthesized in Example 4 and heated to 40° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 190 mg of a light yellow crystal. This crystal was confirmed to be 4,5-dimethyl-3-(2,6-diphenylphenyl)-2(3H)-thiazole-thione by GC-MS. Yield 96%, M⁺=373

### Example 54

### Synthesis of 3-[2,6-di(phenyl)phenyl]-4,5-dimethylthiazolium chloride

A 100 mL flask purged with nitrogen was loaded with 190 mg of 4,5-dimethyl-3-(2,6-diphenylphenyl]-2(3H)-thiazole-thione synthesized in Example 53, 2 g of acetic acid, and 800 mg of 30 wt. % hydrogen peroxide water and the mixture was heated to 60° C. and stirred for 30 minutes.

After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain 180 mg of a light brown powder. The obtained powder was confirmed to be 3-[2,6-di(phenyl)phenyl]-4,5-dimethylthiazolium chloride by ¹H-NMR. Yield 95%

### Example 55

### Synthesis of 3,4,5,6,7-hexahydro-3-(2,6-dibromophenyl)-2H-cycloheptathiazole-2-thione

A 200 mL flask purged with nitrogen was loaded with 5 g of 2,6-dibromoaniline and 10 g of dimethyl sulfoxide and the mixture was cooled to 5° C. while being stirred. The flask was further loaded with a solution obtained by dissolving 800 mg of a sodium hydroxide powder in 700 mg of water and the mixture was stirred for 10 minutes. Further, 1.51 g of carbon disulfide was added for 10 minutes and the mixture was heated to room temperature and stirred for 1 hour and cooled to 5° C. The resulting reaction solution was mixed with 2.92 g of 2-chlorocycloheptanone, heated to room temperature, and stirred for 1 hour. When the resulting reaction solution was mixed with 50 g of water and stirred for 30 minutes, the reaction solution was separated into a gum-like solid and a water layer and therefore, the water layer was removed by decantation and the gum-like solid was washed with 50 g of water. This solid was mixed with 20 g of ethanol and 2 g of concentrated hydrochloric acid and heated and stirred at 60° C. for 30 minutes. After the reaction, the reaction mixture was cooled to room temperature, and the precipitated crystal was filtered and successively washed with 10 g of ethanol and dried to obtain 2.8 g of a light yellow powder. This crystal was confirmed to be 3,4,5,6,7-hexahydro-3-(2,6-dibromophenyl)-2H-cycloheptathiazole-2-thione by GC-MS. Yield 34%, M⁺=419

### Example 56

### Synthesis of 3,4,5,6,7-hexahydro-3-[2,6-bis(3,5-di-tert-butylphenyl)phenyl]-2H-cycloheptathiazole-2-thione

A 100 mL flask purged with nitrogen was loaded with 300 mg of 3,4,5,6,7-hexahydro-3-(2,6-dibromophenyl)-2H-cycloheptathiazole-2-thione synthesized in Example 55, 510 mg of 3,5-di-tert-butylphenylboronic acid, 10 g of tetrahydrofuran, 70 mg of (2-di-tert-butylphosphino)biphenyl, 700 mg of cesium fluoride, and 17 mg of palladium acetate and the mixture was heated to 50° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 440 mg of a light yellow crystal.

This crystal was confirmed to be 3,4,5,6,7-hexahydro-3-[2,6-bis(3,5-di-tert-butylphenyl)phenyl]-2H-cycloheptathiazole-2-thione by GC-MS. Yield 96%, M⁺=638

### Example 57

### Synthesis of 5,6,7,8-tetrahydro-3-[2,6-bis(3,5-di-tert-butylphenyl)phenyl]-4H-cycloheptathiazolium chloride

A 100 mL flask purged with nitrogen was loaded with 440 mg of 3,4,5,6,7-hexahydro-3-[2,6-bis(3,5-di-tert-butylphenyl)phenyl]-2H-cycloheptathiazole-2-thione synthesized in Example 56, 2 g of acetic acid, and 800 mg of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain light brown tar. Since a white powder was precipitated by adding toluene to the tar, the powder was separated by filtration and dried to obtain 250 mg of a white powder. This powder was confirmed to be 5,6,7,8-tetrahydro-3-[2,6-bis(3,5-di-tert-butylphenyl)phenyl]-4H-cycloheptathiazolium chloride by ¹H-NMR. Yield 57%

¹H-NMR (δ/ppm, CDCL3, on the basis of tetramethylsilane): 1.25 (s, 36H), 1.5-2.7 (m, 10H), 6.9-7.7 (m, 9H), 10.99 (s, 1H)

### Example 58

### Synthesis of 3,4,5,6-tetrahydro-3-(2,6-dibromophenyl)-2H-cyclopentathiazole-2-thione

A 200 mL flask purged with nitrogen was loaded with 5 g of 2,6-dibromoaniline and 10 g of dimethyl sulfoxide and the mixture was cooled to 5° C. while being stirred. The flask was further loaded with a solution obtained by dissolving 800 mg of a sodium hydroxide powder in 700 mg of water and the mixture was stirred for 10 minutes. Further, 1.52 g of carbon disulfide was added for 10 minutes and the mixture was heated to room temperature and stirred for 1 hour and cooled to 5° C. The resulting reaction solution was mixed with 2.4 g of 2-chlorocyclopentanone, heated to room temperature, and stirred for 1 hour. When the resulting reaction solution was mixed with 50 g of water and stirred for 30 minutes, the reaction solution was separated into a gum-like solid and a water layer and therefore, the water layer was removed by decantation and the gum-like solid was washed with 50 g of water. This solid was mixed with 20 g of ethanol and 2 g of concentrated hydrochloric acid and heated and stirred at 60° C. for 30 minutes. After the reaction, the reaction mixture was cooled to room temperature, and the precipitated crystal was filtered and successively washed with 10 g of ethanol and dried to obtain 2.1 g of a light brown powder. This crystal was confirmed to be 3,4,5,6-tetrahydro-3-(2,6-dibromophenyl)-2H-cyclopentathiazole-2-thione by GC-MS. Yield 27%, M⁺=319

### Example 59

### Synthesis of 3,4,5,6-tetrahydro-3-[2,6-bis(3,5-di-tert-butylphenyl)phenyl)-2H-cyclopentathiazole-2-thione

A 100 mL flask purged with nitrogen was loaded with 250 mg of 3,4,5,6-tetrahydro-3-(2,6-dibromophenyl)-2H-cyclopentathiazole-2-thione synthesized in Example 58, 432 mg of 3,5-di-tert-butylphenylboronic acid, 10 g of tetrahydrofuran, 60 mg of (2-di-tert-butylphosphino)biphenyl, 600 mg of cesium fluoride, and 14 mg of palladium acetate and the mixture was heated to 60° C. and stirred for 8 hours. At that time, 300 mg of 3,5-di-tert-butylphenylboronic acid was additionally added and the mixture was stirred at 60° C. further for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 350 mg of a light yellow crystal. This crystal was confirmed to be 3,4,5,6-tetrahydro-3-[2,6-bis(3,5-di-tert-butylphenyl)phenyl]-2H-cyclopentathiazole-2-thione by GC-MS. Yield 90%, M⁺=610

### Example 60

### Synthesis of 5,6-dihydro-3-[2,6-bis(3,5-di-tert-butylphenyl)phenyl]-4H-cyclopentathiazolium chloride

A 100 mL flask purged with nitrogen was loaded with 350 mg of 3,4,5,6-tetrahydro-3-[2,6-bis(3,5-di-tert-butylphenyl)phenyl]-2H-cyclopentathiazole-2-thione synthesized in Example 59, 2 g of acetic acid, and 1.0 g of 30 wt. % hydrogen peroxide water and the mixture was heated to 60° C. and stirred for 30 minutes. After the reaction, the solvent was removed by distillation and 10 g of methanol was added to the residue and then methanol was removed. The obtained residue was mixed with 10 g of chloroform and 10 g of saturated salt water and extracted and separated by a separatory funnel. The solvent was removed by distillation from the obtained chloroform layer to obtain light brown tar. Since a white powder was precipitated by adding toluene to the tar, the powder was separated by filtration and dried to obtain 140 mg of a white powder. This powder was confirmed to be 5,6-dihydro-3-[2,6-bis(3,5-di-tert-butylphenyl)phenyl]-4H-cyclopentathiazolium chloride by ¹H-NMR. Yield 40%

¹H-NMR (δ/ppm, CDCL3, on the basis of tetramethylsilane): 1.25 (s, 36H), 1.5-2.8 (m, 6H), 6.8-7.8 (m, 9H), 10.45 (s, 1H)

### Example 61

### Synthesis of 5-tert-butyl-3-(2,6-dibromophenyl)-thiazole-2-thione

A 200 mL flask purged with nitrogen was loaded with 8 g of 2,6-dibromoaniline and 15 g of dimethyl sulfoxide and the mixture was cooled to 5° C. while being stirred. The flask was further loaded with a solution obtained by dissolving 1.28 g of a sodium hydroxide powder in 1100 mg of water and the mixture was stirred for 10 minutes. Further, 2.42 g of carbon disulfide was added for 10 minutes and the mixture was heated to room temperature and stirred for 1 hour and cooled to 5° C. The resulting reaction solution was mixed with 5.6 g of 2-bromo-3,3-dimethylbutylaldehyde, heated to room temperature, and stirred for 1 hour. When the resulting reaction solution was mixed with 50 g of water and stirred for 30 minutes, a crystal was precipitated, and the crystal was separated by filtration and dried to obtain 10.6 g of a light yellow crystal.

A 50 mL flask purged with nitrogen was loaded with 3 g of the crystal, 30 g of toluene, and 170 mg of trifluoroacetic acid, and the mixture was cooled to 0° C. and mixed with 1.55 g of trifluoroacetic anhydride for 10 minutes. The mixed solution was stirred at room temperature for 1 hour. After the reaction, the resulting toluene solution was washed with 10 g of water and concentrated to obtain 2.8 g of a solid material. The solid material obtained by concentration after treatment with a silica gel short column (30 g, elution with 100 g of chloroform) was mixed with methyl-tert-butyl ether and an insoluble crystal was separated by filtration and dried to obtain 1.4 g of a white crystal. This crystal was confirmed to be 5-tert-butyl-3-(2,6-dibromophenyl)-thiazole-2-thione by GC-MS. Yield 49%, M⁺=407

### Example 62

### Synthesis of 5-tert-butyl-3-[2,6-bis(3,5-di-tert-butylphenyl)phenyl]-2(3H)-thiazole-thione

A 100 mL flask purged with nitrogen was loaded with 600 mg of 5-tert-butyl-3-(2,6-dibromophenyl)-thiazole-2-thione synthesized in Example 61, 1.03 g of 3,5-di-tert-butylphenylboronic acid, 20 g of tetrahydrofuran, 132 mg of (2-di-tert-butylphosphino)biphenyl, 1.35 g of cesium fluoride, and 33 mg of palladium acetate and the mixture was heated to 60° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 60 g of silica gel and thereafter, elution with 300 mL of chloroform) and the solvent was removed by distillation to obtain 460 mg of a light yellow oil. This crystal was confirmed to be 5-tert-butyl-3-[2,6-bis[3,5-di-tert-butylphenyl)phenyl]-2(3H)-thiazole-thione by GC-MS. Yield 50%, M⁺=625

### Example 63

### Synthesis of 3-[2,6-bis(3,5-di-tert-butylphenyl)phenyl]-5-tert-butylthiazolium chloride

A 100 mL flask purged with nitrogen was loaded with 460 mg of 3-[2,6-bis(3,5-di-tert-butylphenyl)phenyl]-5-tert-butylthiazolium chloride synthesized in Example 62, 3 g of acetic acid, and 1.5 g of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer to obtain light brown tar. Since a white powder was precipitated by adding methyl-tert-butyl ether to the tar, the powder was separated by filtration and dried to obtain 140 mg of a white powder. This powder was confirmed to be 3-[2,6-bis(3,5-di-tert-butylphenyl)phenyl]-5-tert-butylthiazolium chloride by ¹H-NMR. Yield 30%

¹H-NMR (δ/ppm, CDCL3, on the basis of tetramethylsilane): 1.15-1.35 (6, 45H), 6.9-7.8 (m, 10H), 11.37 (s, 1H) (Example 64

### Synthesis of 4,5,6,7-tetrahydro-7-ethyl-3-(2,6-dibromophenyl)-2(3H)-benzothiazole-thione

A 100 mL flask purged with nitrogen was loaded with 1.2 g of 2,6-dibromoaniline and 6 g of dimethyl sulfoxide and the mixture was cooled to 5° C. while being stirred. The flask was further loaded with a solution obtained by dissolving 200 mg of a sodium hydroxide powder in 200 mg of water and the mixture was stirred for 10 minutes. Further, 370 mg of carbon disulfide was added and the mixture was heated to room temperature and stirred for 1 hour and cooled to 5° C. The resulting reaction solution was mixed with 1.0 g of 2-bromo-3-ethylcyclohexanone, heated to room temperature, and stirred for 1 hour. When the resulting reaction solution was mixed with 10 g of water and stirred for 30 minutes, the reaction solution was separated into a gum-like solid and a water layer and therefore, the water layer was removed by decantation and the gum-like solid was washed with 10 g of water. This solid was mixed with 10 g of ethanol and 1 g of concentrated hydrochloric acid and heated and stirred at 60° C. for 30 minutes. After the reaction, the reaction mixture was cooled to room temperature, and ethanol was removed by distillation. The obtained residue was mixed with 20 g of n-heptane and the precipitated crystal was separated by filtration and dried to obtain 400 mg of a light yellow powder. This crystal was confirmed to be 4,5,6,7-tetrahydro-7-ethyl-3-(2,6-dibromophenyl)-2(3H)-benzothiazole-thione by GC-MS. Yield 19%, M⁺=433

### Example 65

### Synthesis of 4,5,6,7-tetrahydro-7-ethyl-3-[2,6-bis(3,5-di-tert-butylphenyl)phenyl]-2(3H)-benzothiazole-thione

A 100 mL flask purged with nitrogen was loaded with 400 mg of 4,5,6,7-tetrahydro-7-ethyl-3-(2,6-dibromophenyl)-2(3H)-benzothiazole-thione synthesized in Example 64, 650 mg of 3,5-di-tert-butylphenylboronic acid, 10 g of tetrahydrofuran, 84 mg of (2-di-tert-butylphosphino)biphenyl, 850 mg of cesium fluoride, and 21 mg of palladium acetate and the mixture was heated to 60° C. and stirred for 8 hours. After the reaction, the reaction solution was mixed with 10 g of ethyl acetate and 20 g of water and washed and separated by a separatory funnel. The formed organic layer was again washed and separated with 10 g of water and thereafter dried with magnesium sulfate and the solvent was removed by distillation. The obtained residue was refined by a silica gel short column (adsorption in 50 g of silica gel and thereafter, elution with 300 mL of chloroform) and the solvent was removed by distillation to obtain 500 mg of a light yellow crystal. This crystal was confirmed to be 4,5,6,7-tetrahydro-7-ethyl-3-[2,6-bis[3,5-di-tert-butylphenyl)phenyl]-2(3H)-benzothiazole-thione by GC-MS. Yield 83%, M⁺=652 (Example 66

### Synthesis of 3-[2,6-bis(3,5-di-tert-butylphenyl)phenyl]-4,5,6,7-tetrahydro-7-ethyl-benzothiazolium chloride

A 100 mL flask purged with nitrogen was loaded with 480 mg of 4,5,6,7-tetrahydro-7-ethyl-3-[2,6-bis(3,5-di-tert-butylphenyl)phenyl]-2(3H)-benzothiazole-thione synthesized in Example 65, 2 g of acetic acid, and 1 g of 30 wt. % hydrogen peroxide water and the mixture was heated to 60° C. and stirred for 30 minutes. After the reaction, the solvent was removed by distillation and 10 g of methanol was added to the residue and then methanol was removed by distillation. The obtained residue was mixed with 10 g of chloroform and 10 g of saturated salt water and extracted and separated by a separatory funnel. The solvent was removed by distillation from the obtained chloroform layer and the obtained residue was mixed with 10 g of methyl-tert-butyl ether and the precipitated crystal was separated by filtration and dried to obtain 160 mg of a white powder. This powder was confirmed to be 3-[2,6-bis(3,5-di-tert-butylphenyl)phenyl]-4,5,6,7-tetrahydro-7-ethylbenzothiazolium chloride by ¹H-NMR.

Yield 33%

¹H-NMR (δ/ppm, CDCL3, on the basis of tetramethylsilane): 0.90 (t, 3H), 1.23 (s, 36H), 1.2-2.5 (m, 9H), 6.8-7.7 (m, 9H), 11.22 (s, 1H)

### Example 67

A 200 mL Schlenk flask purged with nitrogen was loaded with 6.0 g of 3-methylthiopropanal, 6.9 g of 35 wt. % formalin water, 30 mg of 3-[2,6-bis(3-tert-butylphenyl)phenyl]-4,5,6,7-tetrahydrobenzothiazolium chloride obtained in Example 42, and 12 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 9 mg of 1,8-diazabicyclo[5.4.0]-7-undecene and 100 mg of toluene was added while being stirred and the obtained mixture was stirred at 50° C. for 16 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 26% and 70% of 3-methylthiopropanal, a raw material, was recovered.

The production ratio after 8 hour-reaction time was as follows.

4-(methylthio)-2-oxo-1-butanol: 19%
Raw material 3-methylthiopropanal: 80%
1,6-dimethylthio-4-hydroxy-3-hexanone: 0%

### Example 68

A 200 mL Schlenk flask purged with nitrogen was loaded with 5.8 g of 3-methylthiopropanal, 6.7 g of 35 wt. % formalin water, 30 mg of 3-[2,6-bis(3,5-dichlorophenyl)phenyl]-4,5,6,7-tetrahydrobenzothiazolium chloride obtained in Example 44, and 11 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 9 mg of 1,8-diazabicyclo[5.4.0]-7-undecene and 100 mg of toluene was added while being stirred and the obtained mixture was stirred at 50° C. for 8 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 22% and 65% of 3-methylthiopropanal, a raw material, was recovered.

### Example 69

A 200 mL Schlenk flask purged with nitrogen was loaded with 4.3 g of 3-methylthiopropanal, 4.9 g of 35 wt. % formalin water, 30 mg of 3-[2,6-bis(3,5-dibromophenyl)phenyl]-4,5,6,7-tetrahydrobenzothiazolium chloride obtained in Example 46, and 11 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 7 mg of 1,8-diazabicyclo[5.4.0]-7-undecene and 100 mg of toluene was added while being stirred and the obtained mixture was stirred at 50° C. for 8 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 38% and 60% of 3-methylthiopropanal, a raw material, was recovered.

### Example 70

A 200 mL Schlenk flask purged with nitrogen was loaded with 5.2 g of 3-methylthiopropanal, 5.8 g of 35 wt. % formalin water, 30 mg of 3-[2,6-bis(3,5-di-tert-butylphenyl)-4-methylphenyl]-4,5,6,7-tetrahydrobenzothiazolium chloride obtained in Example 49, and 11 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 10 mg of 1,8-diazabicyclo[5.4.0]-7-undecene and 100 mg of toluene was added while being stirred and the obtained mixture was stirred at 50° C. for 8 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 28% and 70% of 3-methylthiopropanal, a raw material, was recovered.

### Example 71

A 200 mL Schlenk flask purged with nitrogen was loaded with 5.0 g of 3-methylthiopropanal, 5.4 g of 35 wt. % formalin water, 30 mg of 3-[2,6-bis(3,5-di-tert-butylphenyl)-4-chlorophenyl]-4,5,6,7-tetrahydrobenzothiazolium chloride obtained in Example 52, and 11 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 7 mg of 1,8-diazabicyclo[5.4.0]-7-undecene and 100 mg of toluene was added while being stirred and the obtained mixture was stirred at 50° C. for 14 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 56% and 30% of 3-methylthiopropanal, a raw material, was recovered. 1,6-dimethylthio-4-hydroxy-3-hexanone, a homo-coupling product, was produced in an amount of 3%.

### Example 72

A 200 mL Schlenk flask purged with nitrogen was loaded with 12.4 g of 3-methylthiopropanal, 14.5 g of 38 wt. % formalin water, 15 mg of 3-[2,6-bis(3,5-di-tert-butylphenyl)phenyl]-4,5,6,7-tetrahydrobenzothiazolium chloride obtained in Example 13, and 24 g of toluene. After the obtained mixture was heated to 70° C. in a nitrogen atmosphere, a mixed solution containing 7 mg of 1,8-diazabicyclo[5.4.0]-7-undecene and 500 mg of toluene was added while being stirred and the obtained mixture was stirred at 70° C. for 15 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 33% and 65% of 3-methylthiopropanal, a raw material, was recovered.

### Example 73

A 200 mL four-mouth flask equipped with semilunar-shape stirring blades made of Teflon was loaded with 12.2 g of 3-methylthiopropanal, 14.7 g of 38 wt. % formalin water, 16 mg of 3-[2,6-bis(3,5-di-tert-butylphenyl)phenyl]-4,5,6,7-tetrahydro-7-ethyl-benzothiazolium chloride obtained in Example 66, and 25 g of toluene. After the obtained mixture was heated to 70° C. in a nitrogen atmosphere, a mixed solution containing 6 mg of 1,8-diazabicyclo[5.4.0]-7-undecene and 500 mg of toluene was added while being stirred and the obtained mixture was stirred at 70° C. for 15 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 52% and 47% of 3-methylthiopropanal, a raw material, was recovered.

### Example 74

A 200 mL Schlenk flask purged with nitrogen was loaded with 12.4 g of 3-methylthiopropanal, 14.5 g of 38 wt. % formalin water, 45 mg of 3-[2,6-bis(3,5-di-tert-butylphenyl)phenyl]-4,5,6,7-tetrahydrobenzothiazolium chloride obtained in Example 13, and 25 g of toluene. After the obtained mixture was heated to 70° C. in a nitrogen atmosphere, a mixed solution containing 13 mg of 1,8-diazabicyclo[5.4.0]-7-undecene and 500 mg of toluene was added while being stirred and the obtained mixture was stirred at 70° C. for 8 hours. At that time, a mixed solution containing 30 mg of 3-[2,6-bis(3,5-di-tert-butylphenyl)phenyl]-4,5,6,7-tetrahydrobenzothiazolium chloride, 8 mg of 1,8-diazabicyclo[5.4.0]-7-undecene and 330 mg of toluene was added and the obtained mixture was stirred at 70° C. further for 8 hours. After cooled to room temperature, the obtained reaction mixture was liquid-separated into a toluene layer and a water layer. The water layer was extracted three times with 5 g of toluene and the obtained toluene layer was mixed with the initially separated toluene layer to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 67% and 32% of 3-methylthiopropanal, a raw material, remained.

Toluene was removed by distillation from the toluene solution to obtain 14 g of a light yellow oil. Since the oil was solidified when ice-cooled, the oil was mixed with 10 g of toluene and subjected to recrystallization, filtration, and drying to obtain 4.0 g of 4-(methylthio)-2-oxo-butanol in the form of a white platy crystal with a GC purity (area percentage) of 94%. In the same manner, 1.0 g of 4-(methylthio)-2-oxo-1-butanol with a GC purity (area percentage) of 94% was obtained from the filtrate.

### Example 75

A 200 mL Schlenk flask purged with nitrogen was loaded with 5.0 g of 3-methylthiopropanal, 5.7 g of 35 wt. % formalin water, 30 mg of 3-[2,6-bis(3,5-di-tert-butylphenyl)phenyl]-5-tert-butylthiazolium chloride obtained in Example 63, and 10 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 8 mg of 1,8-diazabicyclo[5.4.0]-7-undecene and 100 mg of toluene was added while being stirred and the obtained mixture was stirred at 50° C. for 14 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 20% and 73% of 3-methylthiopropanal, a raw material, was recovered. 1,6-Dimethylthio-4-hydroxy-3-hexanone, a homo-coupling product, was produced in an amount of 1%.

### Example 76

A 200 mL Schlenk flask purged with nitrogen was loaded with 4.8 g of 3-methylthiopropanal, 5.4 g of 35 wt. % formalin water, 30 mg of 5,6,7,8-tetrahydro-3-[2,6-bis(3,5-di-tert-butylphenyl)phenyl]-4H-cycloheptathiazolium chloride obtained in Example 57, and 11 g of toluene. After 500 mg of dry ice was added to the flask, generated gaseous carbon dioxide was discharged to lower the pressure to normal pressure. After the obtained mixture was heated to 50° C. and bubbling of carbon dioxide gas subsided, a mixed solution containing 7 mg of 1,8-diazabicyclo[5.4.0]-7-undecene and 100 mg of toluene was added while being stirred and the obtained mixture was stirred at 50° C. for 8 hours. The obtained reaction mixture was cooled to room temperature to obtain a reaction mixture containing 4-(methylthio)-2-oxo-1-butanol. According to analysis by a gas chromatography internal standard method, the yield of 4-(methylthio)-2-oxo-1-butanol, a cross-coupling product, was 17% and 82% of 3-methylthiopropanal, a raw material, was recovered.

### Example 77

### Observation of 3-[2,6-bis(3,5-di-tert-butylphenyl)phenyl]-4,5,6,7-tetrahydrobenzothiazol-2-ylidene by NMR

In a glove box in a nitrogen atmosphere, an NMR tube was loaded with 11 mg of 3-[2,6-bis(3,5-di-tert-butylphenyl)phenyl]-4,5,6,7-tetrahydrobenzothiazolium chloride obtained in Example 13. The NMR tube was further loaded with a mixed solution obtained by dispersing 10 mg of sodium-tert-butoxide in 0.7 mL of deuterated toluene and tightly closed.

The obtained mixture was subjected to ¹H-NMR measurement at room temperature. After 10 minutes, formation of a new peak of 3-[2,6-bis(3,5-di-tert-butylphenyl)phenyl]-4,5,6,7-tetrahydrobenzothiazol-2-ylidene started and after 3 hours, the peak of 3-[2,6-bis(3,5-di-tert-butylphenyl)phenyl]-4,5,6,7-tetrahydrobenzothiazolium chloride almost completely disappeared and the main peak was changed to the peak of 3-[2,6-bis(3,5-di-tert-butylphenyl)phenyl]-4,5,6,7-tetrahydrobenzothiazol-2-ylidene. It was observed characteristically that the proton at 2nd position of the thiazolium ring disappeared; the proton of tert-butyl appeared in 2 separate lines; and protons at 2nd and 6th positions of 3,5-di-tert-butylphenyl were shifted to lower magnetic fields.

¹H-NMR (δ/ppm, CD3C6D5, on the basis of deuterated toluene methyl group): 1.40 (s, 18H), 1.45 (s, 18H), 1.5-1.8 (m, 8H), 7.19 (t, 1H), 7.40 (d, 1H), 7.42 (dd, 1H), 7.52 (t, 1H), 7.54 (dd, 1H), 7.55 (t, 1H), 8.10 (d, 1H)

In the case NMR measurement was carried out after several 10 mg of acetaldehyde was added to the NMR tube which was subjected to the measurement after 3 hours, it was confirmed that the peak of 3-[2,6-bis(3,5-di-tert-butylphenyl)phenyl]-4,5,6,7-tetrahydrobenzothiazol-2-ylidene almost completely disappeared and 3-hydroxy-2-butanone, a homo-coupling product of acetaldehyde, was produced at a high yield.

### INDUSTRIAL APPLICABILITY

The invention provides an innovative method for producing an α-hydroxy ketone compound. The invention is advantageous in terms of improvement of selectivity in production of an α-hydroxy ketone compound per unit catalyst amount.

1. A method for producing an α-hydroxy ketone compound by carrying out a coupling reaction of an aldehyde compound in the presence of a base compound and a thiazolium salt defined by a formula (1) 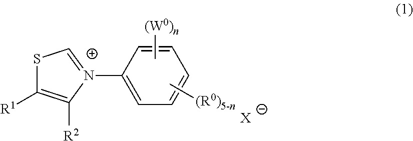
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; X⁻ denotes an anion; n denotes 1 or 2; in the case n is 2, 2 groups denoted by W⁰ may be mutually the same or different; and all or some of a plurality of groups denoted by R⁰ may be the same or different).

2. The method as described in claim 1, wherein the thiazolium salt defined by a formula (1) is a thiazolium salt defined by a formula (1′) 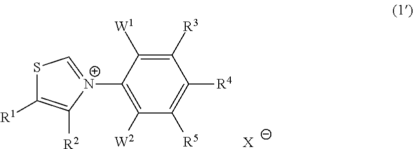
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; W¹ and W² independently denote an aryl which may have a substituent or a halogen atom; and X— denotes an anion).

3. The method as described in claim 1, wherein the thiazolium salt defined by a formula (1) is a thiazolium salt defined by a formula (1″) 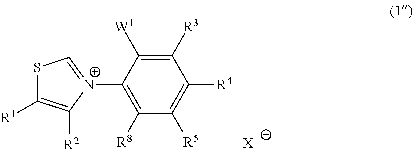
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; W¹ denotes an aryl which may have a substituent or a halogen atom; and X— denotes an anion).

4. A method for producing an α-hydroxy ketone compound by carrying out a coupling reaction of an aldehyde compound in the presence of thiazol-2-ylidene defined by a formula (1-2) 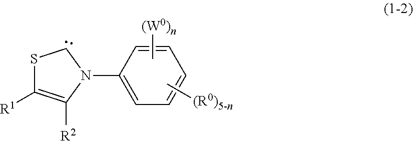
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; n denotes 1 or 2; in the case n is 2, 2 groups denoted by W⁰ may be mutually the same or different; and all or some of a plurality of groups denoted by R⁰ may be the same or different).

5. The method as described in claim 4, wherein the thiazol-2-ylidene defined by a formula (1-2) is a thiazol-2-ylidene defined by a formula (1′-2) 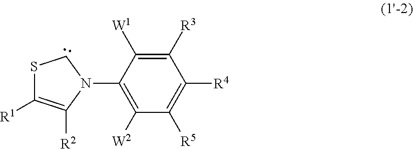
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; and W¹ and W² independently denote an aryl which may have a substituent or a halogen atom).

6. The method as described in claim 4, wherein the thiazol-2-ylidene defined by a formula (1-2) is a thiazol-2-ylidene defined by a formula (1″-2) 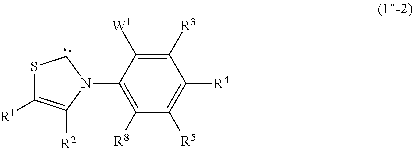
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; and W¹ denotes an aryl which may have a substituent or a halogen atom).

7. The method as described in claim 1, wherein the coupling reaction of an aldehyde compound is carried out in the presence of carbon dioxide.

8. The method as described in claim 1, wherein the base compound is at least one kind of compound selected from the group consisting of organic bases, alkali metal salts, and alkaline earth metal salts.

9. The method as described in claim 1, wherein the coupling reaction of an aldehyde compound is a homo-coupling reaction of an aldehyde compound defined by a formula (2) 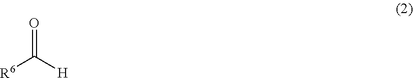
(wherein R⁶ denotes a hydrogen atom, an alkyl which may have a substituent, an aryl which may have a substituent, or a heteroaryl which may have a substituent).

10. The method as described in claim 1, wherein the coupling reaction of an aldehyde compound is a cross-coupling reaction of an aldehyde compound defined by a formula (2) 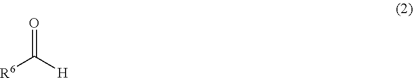
(wherein R⁶ denotes a hydrogen atom, an alkyl which may have a substituent, an aryl which may have a substituent, or a heteroaryl which may have a substituent) and an aldehyde compound defined by a formula (4) 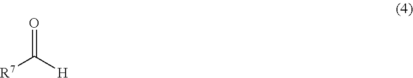
(wherein R⁷ is different from R⁶ and denotes a hydrogen atom, an alkyl which may have a substituent, an aryl which may have a substituent, or a heteroaryl which may have a substituent).

11. The method as described in claim 10, wherein R⁶ denotes an alkyl which may have a substituent and R⁷ denotes a hydrogen atom.

12. The method as described in claim 11, wherein the aldehyde compound defined by the formula (4) is formaldehyde co-existing with water.

13. The method as described in claim 12, wherein the coupling reaction of an aldehyde compound is carried out in the presence of a solvent having no compatibility with water.

14. The method as described in claim 10, wherein the aldehyde compound defined by the formula (2) is 3-methylthiopropanal and the α-hydroxy ketone compound is 4-methylthio-2-oxo-1-butanol.

15. A thiazolium salt defined by a formula (1) 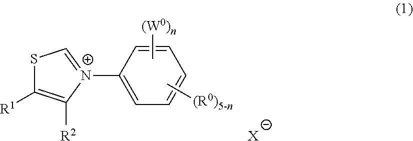
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent;
W⁰ denotes an aryl which may have a substituent or a halogen atom; X⁻ denotes an anion; n denotes 1 or 2; in the case n is 2, 2 groups denoted by W⁰ may be mutually the same or different; and all or some of plurality of groups denoted by R⁰ may be the same).

16. The thiazolium salt as described in claim 15, wherein the thiazolium salt defined by a formula (1) is a thiazolium salt defined by a formula (1′) 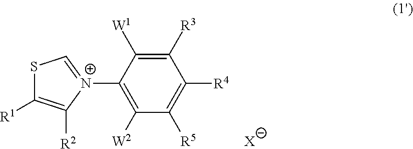
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; W¹ and W² independently denote an aryl which may have a substituent or a halogen atom; and X⁻ denotes an anion).

17. The thiazolium salt as described in claim 16, wherein R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom, a halogen atom, or a C₁₋₁₀ alkyl; and
W¹ and W² independently denote a C₆₋₁₀ aryl which may have a substituent.

18. The thiazolium salt as described in claim 15, wherein the thiazolium salt defined by a formula (1) is a thiazolium salt defined by a formula (1″) 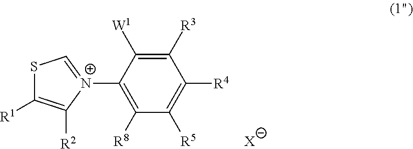
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; W¹ denotes an aryl which may have a substituent or a halogen atom; and X⁻ denotes an anion).

19. The thiazolium salt as described in claim 18, wherein R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom or a C₁₋₁₀ alkyl;
R⁸ denotes a C₁₋₁₀ alkyl; and
W¹ denotes a C₆₋₁₀ aryl which may have a substituent.

20. The thiazolium salt as described in claim 15, wherein the thiazolium salt defined by a formula (1) is 3-[2,6-di(phenyl)phenyl]-4,5-dimethylthiazolium salt, 3-[2,6-bis(3,5-di-tert-butylphenyl)phenyl]-4,5-dimethylthiazolium salt, 3-[(2,6-diphenyl-3,5-diisopropyl)phenyl]-4,5-dimethylthiazolium salt, 3-[2,6-bis(3,5-di-tert-butylphenyl)phenyl]-4,5,6,7-tetrahydrobenzothiazolium salt, 3-[2,6-bis(3,5-di-tert-butylphenyl)-4-chlorophenyl]-4,5,6,7-tetrahydrobenzothiazolium salt, 3-[2-(3,5-di-tert-butylphenyl)-6-methylphenyl]-4,5,6,7-tetrahydrobenzothiazolium salt, or 3-[2,6-bis(3,5-di-tert-butylphenyl)phenyl]-4,5,6,7-tetrahydro-7-ethylbenzothiazolium salt.

21. A thiazol-2-ylidene defined by a formula (1-2) 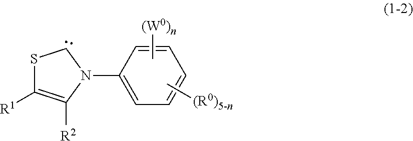
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; W⁰ denotes an aryl which may have a substituent or a halogen atom; n denotes 1 or 2; in the case n is 2, 2 groups denoted by W⁰ may be mutually the same or different; and all or some of a plurality of groups denoted by R⁰ may be the same).

22. The thiazol-2-ylidene as described in claim 21, wherein the thiazol-2-ylidene defined by a formula (1-2) is a thiazol-2-ylidene defined by a formula (1′-2) 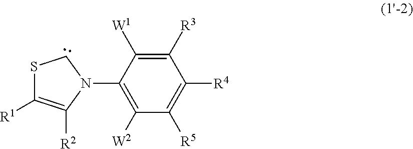
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; and W¹ and W² independently denote an aryl which may have a substituent or a halogen atom).

23. The thiazol-2-ylidene as described in claim 22, wherein R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom, a halogen atom, or a C₁₋₁₀ alkyl; and
W¹ and W² independently denote a C₆₋₁₀ aryl which may have a substituent.

24. The thiazol-2-ylidene as described in claim 21, wherein the thiazol-2-ylidene defined by a formula (1-2) is a thiazol-2-ylidene defined by a formula (1″-2) 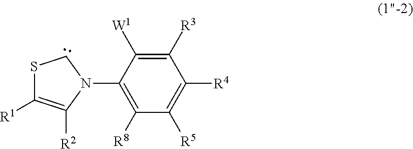
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; and W¹ denotes an aryl which may have a substituent or a halogen atom).

25. The thiazol-2-ylidene as described in claim 24, wherein R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom or a C₁₋₁₀ alkyl;
R⁸ denotes a C₁₋₁₀ alkyl; and
W¹ denotes a C₆₋₁₀ aryl which may have a substituent.

26. The thiazol-2-ylidene as described in claim 21, wherein the thiazol-2-ylidene defined by a formula (I-2) is 3-[2,6-di(phenyl)phenyl]-4,5-dimethylthiazol-2-ylidene, 3-[2,6-bis(3,5-di-tert-butylphenyl)phenyl]-4,5-dimethylthiazol-2-ylidene, 3-[(2,6-diphenyl-3,5-diisopropyl)phenyl]-4,5-dimethylthiazol-2-ylidene, 3-[2,6-bis(3,5-di-tert-butylphenyl)phenyl]4,5,6,7-tetrahydrobenzothiazol-2-ylidene, 3-[2,6-bis(3,5-di-tert-butylphenyl)-4-chlorophenyl]-4,5,6,7-tetrahydrobenzothiazol-2-ylidene, 3-[2-(3,5-di-tert-butylphenyl)-6-methylphenyl]-4,5,6,7-tetrahydrobenzothiazol-2-ylidene, or 3-[2,6-bis(3,5-di-tert-butylphenyl)phenyl]-4,5,6,7-tetrahydro-7-ethylbenz othiazol-2-ylidene.

27. A method for producing a thiazolium salt defined by a formula (8) 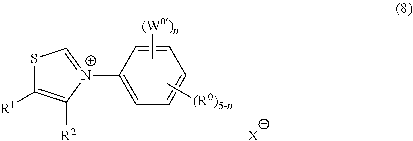
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R⁰ denotes a hydrogen atom, a halogen atom, an alkyl which may have a substituent, or an aryl which may have a substituent; all or some of 4 groups denoted by R⁰ may be the same; W^{0′} denotes an aryl which may have a substituent; X⁻ denotes an anion; and n denotes the same as above) by carrying out a step A of obtaining a 3-(aryl-substituted aryl)-2-thiazole-thione compound defined by a formula (7) 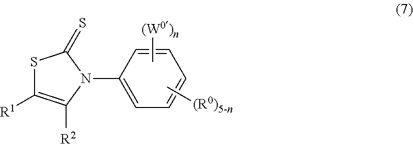
(wherein R¹, R², R⁰, W^{0′}, and n respectively denote the same as described above) by a coupling reaction of a 3-(halo-substituted aryl)-2-thiazole-thione compound defined by a formula (6) 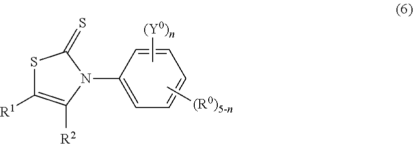
(wherein R¹, R², R⁰, and X⁻ respectively denote the same as described above; and Y⁰ denotes a halogen atom)
and an aryl compound defined by a formula (5)
W^{0′}-L (5)
(wherein W0′ denotes the same as described above; and L denotes a leaving group) in the presence of a palladium catalyst and a step B of oxidizing the 3-(aryl-substituted aryl)-2-thiazole-thione compound defined by the formula (7) and obtained in the step A.

28. The method as described in claim 27, wherein L is —B(OH)₂ or —MgX⁰ (wherein X⁰ denotes a halogen atom).

29. The method as described in claim 27, wherein L is —B(OH)₂ and the coupling reaction in the step A is carried out in the presence of a base compound.

30. The method as described in claim 27, wherein L is —MgX⁰ (wherein X⁰ denotes a halogen atom) and the coupling reaction in the step A is carried out in the presence of a zinc compound.

31. A thiazole-thione compound defined by a formula (7′) 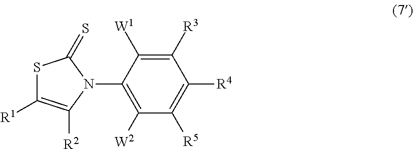
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; and W¹ and W² independently denote an aryl which may have a substituent or a halogen atom).

32. The thiazole-thione compound as described in claim 31, wherein R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom, a halogen atom, or a C₁₋₁₀ alkyl; and
W¹ and W² independently denote a C₆₋₂₀ aryl.

33. The thiazole-thione compound as described in claim 31, wherein the thiazole-thione compound defined by a formula (7′) is a thiazole-thione compound defined by a formula (7″) 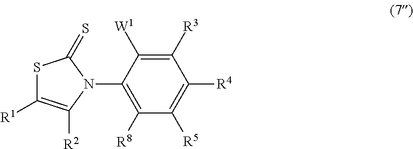
(wherein R¹ and R² independently denote a hydrogen atom, an alkyl which may have a substituent, an alkoxycarbonyl which may have a substituent, an alkylcarbonyl which may have a substituent, or an aryl which may have a substituent, or R¹ and R² may be bonded to each other to form a ring together with the carbon atoms bonded to R¹ and R²; R³, R⁴, and R⁵ independently denote a hydrogen atom, a halogen atom, an alkyl which may have a substituent, or an aryl which may have a substituent; R⁸ denotes a hydrogen atom or an alkyl which may have a substituent; and W¹ denotes an aryl which may have a substituent or a halogen atom).

34. The thiazole-thione compound as described in claim 33, wherein R¹ and R² independently denote a C₁₋₁₀ alkyl, or R¹ and R² are bonded to each other to form a C₅₋₁₀ cycloalkene ring together with the carbon atoms bonded to R¹ and R²;
R³, R⁴, and R⁵ independently denote a hydrogen atom or a C₁₋₁₀ alkyl;
R⁸ denotes a C₁₋₁₀ alkyl; and
W¹ denotes a C₆₋₂₀ aryl.
